(19) United States
(12) Patent Application Publication
(10) Pub. No.: US 2025/0144215 A1
(43) Pub. Date: May 8, 2025

(54) METHOD FOR ENRICHING TUMOR INFILTRATING LYMPHOCYTES

(71) Applicant: ABELZETA INC., Rockville, MD (US)

(72) Inventors: Xiaobing LUO, Rockville, MD (US); Fei TANG, Rockville, MD (US); Michael PATRICK, Rockville, MD (US); Thu Le TRINH, Rockville, MD (US); Jiaqi HUANG, Rockville, MD (US); Xin YAO, Rockville, MD (US); Shigui ZHU, Rockville, MD (US); Yihong YAO, Rockville, MD (US)

(73) Assignee: ABELZETA INC., Rockville, MD (US)

(21) Appl. No.: 18/832,664

(22) PCT Filed: Jan. 27, 2023
(86) PCT No.: PCT/US2023/061497

§ 371 Date: Jul. 24, 2024

Related U.S. Application Data
(60) Provisional application No. 63/304,288, filed on Jan. 28, 2022.
(60) Provisional application No. 63/411,317, filed on Sep. 29, 2022.
(60) Provisional application No. 63/423,676, filed on Nov. 8, 2022.

(51) Int. Cl. A61K 40/42; A61K 40/11; C12N 5/0783

(52) U.S. Cl. CPC A61K 40/428; A61K 40/11; C12N 5/0636; C12N 2501/2302; C12N 2502/30

(57) ABSTRACT

The present disclosure provides for improved methods for expanding tumor infiltrating lymphocytes (TILs) and producing therapeutic populations of TILs in a shorter time period than the traditional methods. TIL cell therapy products can be manufactured which easily meet the target cell dose while maintaining desired T cell phenotype.

### CROSS REFERENCE TO RELATED APPLICATIONS

This application claims priority to U.S. Provisional Application Nos. 63/304,288 (filed on Jan. 28, 2022), 63/411,317 (filed on Sep. 29, 2022), and 63/423,676 (filed on Nov. 8, 2022), each of which is incorporated herein by reference in its entirety.

### BACKGROUND

Adoptive cell therapy with tumor-infiltrating lymphocytes (TILs) offers a potential therapeutic option for cancer. TILs are enriched with polyclonal T cells with diverse antigen specificity. Extraction of a fragment of tumor followed by ex vivo expansion removes TILs from the hostile tumor microenvironment and reduces the immunosuppressive effects of intratumoral regulatory T cells. Expansion of TILs ex vivo rejuvenates the cells, yielding billions of such cells to be infused back into the patient. A cellular therapy product that can address the broad nature of tumor neoantigens and the unique array from each patient would lead to the possibility of a tailored response. Sarnaik et al., J. Clinical Oncology, 2021, 39:2656-2666.

Effective treatment of cancer using TILs requires the ex vivo production of billions of TILs. For TIL manufacturing, resected patient tumors are first fragmented and cultured in vitro under conditions that support the migration of TILs from the tumor tissue. In the conventional protocols, TIL numbers are then expanded in a rapid expansion protocol (REP) using irradiated allogeneic donor peripheral blood mononuclear cells (PBMCs) and anti-CD3 monoclonal antibodies. REP is used to generate sufficient TILs for infusion. Several factors, including availability and validation of donor PBMCs, make conventional REP protocols time-consuming, laborious, and expensive. In addition to these detracting factors, TILs expanded with standard protocols primarily exhibit a terminally differentiated phenotype (T_{EM}, T_{EMRA}) which is associated with poorer clinical responses. A young memory phenotype (T_{SCM}, T_{CM}) in TIL infusion products has been linked to improved anti-tumor responses in patients. Therefore, there is a need to replace the conventional REP protocol with a more facile yet GMP-compliant process capable of producing TILs with a favorable phenotype while using readily available commercial reagents.

### SUMMARY

The present disclosure provides for a method of expanding tumor-infiltrating lymphocytes (TILs), the method comprising: (a) culturing a first population of cells in a first cell culture medium to generate a second population of cells; and (b) contacting the second population of cells with a polymeric matrix comprising anti-CD3 and anti-CD28 antibodies or fragments thereof in a second cell culture medium, to generate a third population of cells. In certain embodiments, the first population of cells is obtained from a tumor sample from a patient.

The third population of cells may be the TILs or cells to be included in the present pharmaceutical compositions. The third population of cells may be the TILs or cells to be used in the present methods to treat a subject (e.g., a patient) with cancer.

In certain embodiments, the second cell culture medium comprises about 100 IU/mL to about 8,000 IU/mL interleukin-2 (IL-2), about 500 IU/mL to about 4,000 IU/mL IL-2, about 1,000 IU/mL, about 2,000 IU/mL, about 3,000 IU/mL, about 4,000 IU/mL, or about 5,000 IU/mL IL-2.

In certain embodiments, the first cell culture medium comprises about 2,000 IU/mL to about 8,000 IU/mL IL-2, about 5,000 IU/mL, about 6,000 IU/mL, about 7,000 IU/mL, or about 8,000 IU/mL IL-2.

In certain embodiments, in step (a) the first population of cells is cultured for about 10 days to about 40 days, for about 10 days to about 14 days, for about 10 days, for about 11 days, for about 12 days, for about 13 days, for about 14 days, for about 15 days, for about 16 days, or for about 17 days.

The method may further comprise cryopreserving the second population of cells after step (a).

In certain embodiments, in step (b) the contacting is for about 3 days to about 17 days, for about 5 days to about 15 days, for about 10 days, for about 11 days, for about 12 days, for about 13 days, for about 14 days, for about 15 days, for about 16 days, or for about 17 days.

In certain embodiments, the third population of cells is at least 100-fold greater in number than the second population of cells, or is about 100-fold to about 2000-fold greater in number than the second population of cells.

The tumor sample may be from a solid tumor. Solid tumors include, but are not limited to, a sarcoma, hepatocellular carcinoma, glioma, head-neck cancer, bone cancer, brain cancer, breast cancer, cancer of the anus, cancer of the anal canal, cancer of the anorectum, cancer of the eye, cancer of the intrahepatic bile duct, cancer of the joints, cancer of the neck, cancer of the gallbladder, cancer of the pleura, cancer of the nose, cancer of the nasal cavity, cancer of the middle ear, cancer of the oral cavity, cancer of the vulva, colon cancer, esophageal cancer, cervical cancer, gastrointestinal cancer, hypopharynx cancer, larynx cancer, liver cancer, lung cancer, malignant mesothelioma, melanoma, nasopharynx cancer, ovarian cancer, pancreatic cancer, peritoneum cancer, omentum cancer, mesentery cancer, pharynx cancer, prostate cancer, rectal cancer, renal cancer, small intestine cancer, soft tissue cancer, stomach cancer, testicular cancer, thyroid cancer, ureter cancer, urinary bladder cancer, and combinations thereof.

The present disclosure provides for tumor-infiltrating lymphocytes obtained by the present method.

Also encompassed by the present disclosure is a cell population enriched for, or expanded from, tumor-infiltrating lymphocytes, comprising one or more of the following:
(i) CD3⁺ CD8⁺ T cells at a percentage ranging from about 3% to about 88% of CD3⁺ cells,
(ii) CD3⁺ CD4⁺ T cells at a percentage ranging from about 10% to about 96% of CD3⁺ cells,
(iii) CD4⁺ T_{CM} T cells at a percentage ranging from about 50% to about 88% of CD4⁺ cells,
(iv) CD8⁺ T_{CM} T cells at a percentage ranging from about 28% to about 82% of CD8⁺ cells, (v) CD4⁺ T_{EM} T cells at a percentage ranging from about 11% to about 49% of CD4⁺ cells, and (vi) CD8⁺ T_{EM} T cells at a percentage ranging from about 11% to about 61% of CD8⁺ cells, where the cell population comprises no less than 70% of live cells, and where the cell population is generated from a tumor sample from a patient.

The cell population may comprise no less than 80% CD3⁺ T cells in live cells.

The cell population may comprise CD4⁺ CD27⁺ T cells at a percentage ranging from about 10% to about 51% of CD4⁺ cells.

The cell population may comprise CD8⁺ CD27⁺ T cells at a percentage ranging from about 12% to about 72% of CD8⁺ cells.

The cell population may comprise CD8⁺ CD28⁺ T cells at a percentage ranging from about 34% to about 95% of CD8⁺ cells.

The cell population may comprise CD4⁺ CD28⁺ T cells at a percentage ranging from about 82% to about 100% of CD4⁺ cells.

The cell population may comprise CD4⁺ 4-1BB⁺ T cells at a percentage ranging from about 0.2% to about 5.8% of CD4⁺ cells.

The cell population may comprise CD8⁺ 4-1BB⁺ T cells at a percentage ranging from about 0.2% to about 11.6% of CD8⁺ cells.

The cell population may comprise CD4⁺ LAG3⁺ T cells at a percentage ranging from about 0.2% to about 19.5% of CD4⁺ cells.

The cell population may comprise CD8⁺ LAG3⁺ T cells at a percentage ranging from about 6% to about 51.2% of CD8⁺ cells.

The cell population may comprise CD4⁺ PD1⁺ T cells at a percentage ranging from about 0.9% to about 31% of CD4⁺ cells.

The cell population may comprise CD8⁺ PD1⁺ T cells at a percentage ranging from about 1% to about 18% of CD8⁺ cells.

The cell population may comprise no greater than 10% CD56⁺ NK cells.

The present disclosure provides for a cell population obtained by the present method.

The method may comprise: (a) culturing a first population of cells in a first cell culture medium to generate a second population of cells; and (b) contacting the second population of cells with a polymeric matrix comprising anti-CD3 and anti-CD28 antibodies or fragments thereof in a second cell culture medium, to generate a third population of cells. In certain embodiments, the first population of cells is obtained from a tumor sample from a patient. The third population of cells may be cell population generated by the present method.

The present disclosure provides for a method of expanding a cell population enriched for tumor-infiltrating lymphocytes. The method may comprise: (a) culturing cells obtained from a tumor sample from a patient; (b) treating the cultured cells to generate a cell population enriched for tumor-infiltrating lymphocytes, the cell population comprising one or more of the following:
(i) CD3⁺ CD8⁺ T cells at a percentage ranging from about 3% to about 88% of CD3⁺ cells,
(ii) CD3⁺ CD4⁺ T cells at a percentage ranging from about 10% to about 96% of CD3⁺ cells,
(iii) CD4⁺ CD45RA⁻CD62L⁺ central memory T cells at a percentage ranging from about 50% to about 88% of CD4⁺ cells,
(iv) CD8⁺CD45RA⁻CD62L⁺ central memory T cells at a percentage ranging from about 28% to about 82% of CD8⁺ cells,
(v) CD4⁺ CD45RA⁻CD62L⁻ effector memory T cells at a percentage ranging from about 11% to about 49% of CD4⁺ cells, and
(vi) CD8⁺ T CD45RA⁻CD62L⁻ effector memory T cells at a percentage ranging from about 11% to about 61% of CD8⁺ cells.

Also encompassed by the present disclosure is a method of treating a patient with cancer, the method comprising administering to the patient the present tumor-infiltrating lymphocytes, the present cell population, the present cells (e.g., the third population of cells) or present composition.

The present disclosure provides for a pharmaceutical composition comprising the present tumor-infiltrating lymphocytes, the present cell population, or the present cells (e.g., the third population of cells).

In certain embodiments, about 1×10⁹ to about 1×10¹¹ cells, or about 5×10⁹ to about 9×10¹⁰ cells, are administered to the patient.

The cancer may be melanoma, cervical cancer, lung cancer, colorectal cancer, breast cancer, or head and neck cancer. The cancer may comprise a sarcoma, hepatocellular carcinoma, glioma, head-neck cancer, bone cancer, brain cancer, breast cancer, cancer of the anus, cancer of the anal canal, cancer of the anorectum, cancer of the eye, cancer of the intrahepatic bile duct, cancer of the joints, cancer of the neck, cancer of the gallbladder, cancer of the pleura, cancer of the nose, cancer of the nasal cavity, cancer of the middle ear, cancer of the oral cavity, cancer of the vulva, colon cancer, esophageal cancer, cervical cancer, gastrointestinal cancer, hypopharynx cancer, larynx cancer, liver cancer, lung cancer, malignant mesothelioma, melanoma, nasopharynx cancer, ovarian cancer, pancreatic cancer, peritoneum cancer, omentum cancer, mesentery cancer, pharynx cancer, prostate cancer, rectal cancer, renal cancer, small intestine cancer, soft tissue cancer, stomach cancer, testicular cancer, thyroid cancer, ureter cancer, urinary bladder cancer, or combinations thereof.

### BRIEF DESCRIPTION OF THE DRAWINGS

FIG. 1: TIL expansion and glucose/lactate levels in pre-REP TIL cultures following the standardized protocol. In the standardized pre-REP protocol tumor fragments were placed in a G-Rex® 100M device containing 250 mL of pre-REP complete medium, fed 1 volume of medium on Day 10, and harvested on Day 14. Supernatants were sampled on Days 0, 6, 10, and 14.

FIG. 2: Lineage characterization of pre-REP TILs from the standardized protocol. NK, CD45⁺CD3⁻CD56⁺, among CD45⁺ cells; B, CD45⁺CD3⁻CD19⁺, among CD45⁺ cells; myeloid, CD45⁺CD3⁻CD14⁺, among CD45⁺ cells; epithelial, CD45⁻EPCAM⁺, among live cells; CD3⁺ T cells, among CD45⁺ cells; CD4⁺ T and CD8⁺ T, among CD3⁺ T cells.

FIG. 3: Expression of activation/inhibitory surface markers and subsets of CD4⁺ pre-REP TILs generated following the standardized protocol.

FIG. 4: Expression of activation/inhibitory surface markers and subsets of CD8⁺ pre-REP TILs generated following the standardized protocol.

FIG. 5: Percentage of CD3⁺ T cells, and CD4⁺ and CD8⁺ T Cell Subsets in TILs Expanded by Traditional REP or TransAct REP. Statistical significance was determined by paired t test: ** P<0.01; ns, not significant (n=12).

FIG. 6: Memory Phenotype of CD4⁺ and CD8⁺ TILs. Top panel: CD4, Bottom panel: CD8. Each dot represents one donor. Horizontal bars represent the mean and standard deviation. T_{N}, naïve T cells; T_{SCM}, stem cell memory; T_{CM}, central memory; T_{EM}, effector memory; T_{EFF}, effector T cells. P values were generated by Student's paired t test. Ns, not significant; * P<0.05; ** P<0.01; *** P<0.001; ****P<0.0001 (n=12).

FIG. 7: Analysis of Inhibitory and Activation Markers on CD4⁺ and CD8⁺ TIL Subsets. Top panel: CD4, Bottom panel: CD8. Each dot represents one donor. Horizontal bars represent the mean and standard deviation. P values were generated by Student's paired t test. ns, not significant; * P<0.05; ** P<0.01; *** P<0.001 (n=12).

FIG. 8: IFN-γ and Granzyme B Production in Response to αCD3 Stimulation. The stimulation was performed in triplicates for all the donors and each dot represents their average values. Horizontal bars represent the grand mean of different donors in each condition. P values were generated by paired t test. ns, not significant; * P<0.05; ** P<0.01.

FIG. 9: Tumor Killing Effects of C-TIL051 (TransAct REP TILs) and Traditional REP TILs during Incubation with Autologous Tumor Cells. TILs from Donor T5101016 were labeled with Cytolight Rapid Red then co-cultured with cancer MOS at E:T ratios of 2:1, 3:1 and 4:1 in culture media containing Caspase 3/7 Green for 68 hours. MOS alone served as negative control. Higher signal of Caspase3/7 indicated more killing.

FIG. 10: The Dynamic Changes of IL-2 Protein Concentrations in C-TIL051 REP PD Run Cultures Using TransAct and G-Rex® Devices. The IL-2 protein concentrations in the REP CM (Day 0) or culture supernatant samples collected at various time points were measured by ELISA (left). Samples from 7 REP runs were analyzed, in which TILs of different donors displayed varying levels of expansion (right; Day 14 data represent projected number after considering respective split factor on Day 9).

FIG. 11: The Dynamic Changes of Glucose and Lactate Levels in C-TIL051 REP PD Run Cultures Using TransAct and G-Rex® Devices. Glucose and lactate concentrations in the REP CM (Day 0) or culture supernatant samples collected at various time points were measured by Glucose-Glo Assay and Lactate-Glo Assay, respectively. Samples from 7 REP runs were analyzed, in which TILs of different donors displayed varying levels of expansion.

FIG. 12: FACS Analysis of Surface Marker Expression and Memory Phenotype of CD4⁺ and CD8⁺ TILs after REP with TransAct Using G-Rex® Devices. Data are presented as mean with 95% CI. T_{N} (Tnaive), naive T cells; T_{SCM} (Tscm), stem memory T cells; T_{CM} (T_{CM}), central memory T cells; T_{EM} (Tem), effector memory T cells; T_{EFF}, effector T cells.

FIG. 13: The Dynamic Changes of IL-2 Protein Concentrations in C-TIL051 REP PD Run Cultures with TransAct and Sequential Use of a G-Rex® Device and Xuri™ W25 Bioreactor. After sampling of supernatant from the G-Rex® culture on Day 7, the culture was inoculated to Xuri™ Cellbag on the same day and then sampled daily from Day 8 until harvest.

FIG. 14: The Dynamic Changes of Glucose and Lactate Concentrations in C-TIL051 REP PD Run Cultures with TransAct and Sequential Use of a G-Rex® Device and Xuri® W25 Bioreactor. After sampling of supernatant from the G-Rex® culture on Day 7, the culture was inoculated to Xuri™ Cellbag on the same day and then sampled daily from Day 8 until harvest.

FIG. 15: Cell Proliferation, Viability, and Cumulative Expansion Fold of REP TILs from C-TIL051 PD Runs with TransAct and Sequential Use of G-Rex® Devices and Xuri™ W25 Bioreactor. Five pre-REP samples were first activated with TransAct and expanded in a G-Rex® 100M device and then transferred to a Xuri™ W25 bioreactor on Day 7. Cells were further expanded for 8-12 days on Xuri™ with daily sampling for cell proliferation and viability.

FIG. 16: FACS Analysis of Surface Marker Expression and Memory Phenotype of CD4⁺ and CD8⁺ TILs after REP with TransAct and Sequential Use of G-Rex® Device and Xuri™ W25 Bioreactor. Data are presented as mean with 95% CI. T_{N}, naive T cells; T_{SCM}, stem memory T cells; T_{CM}, central memory T cells; T_{EM}, effector memory T cells; T_{EFF}, effector T cells.

FIG. 17: IFN-γ Production by C-TIL051 from Mock Runs in Response to αCD3/CD28 Stimulation.

FIG. 18: Upregulation of T Cell Activation Markers (4-1BB and OX40) by C-TIL051 Engineer Runs in Response to αCD3 Stimulation. The horizontal line indicates grand mean.

FIG. 19: Evaluation of C-TIL051 Mediated MOS Killing Using the T Cell Potency Assay. Quantification of relative T5101034 (left) and T5101035 (right) autologous TIL-induced MOS killing observed at E:T ratio of 1:1, 5:1, and 10:1. Co-culture was started on day 3 post-establishment of MOS, and images were obtained up to 96 hours. The average integrated fluorescence intensity of the NIR channel (NIRCU×μm²) was from viable tumor cells labeled with NIR680 dye. Data were normalized to the MOS only group.

FIG. 20: Requirement of HLA Engagement Demonstrated the Specificity of C-TIL051 Mediated MOS Killing. Co-culture of the C-TIL051 REP TILs with the autologous MOS were performed at effector: target ratio of 5:1, with or without indicated HLA blocking antibodies, and analyzed up to 96 hours. The average integrated fluorescence intensity of the NIR channel (NIRCU×μm²) were from viable tumor cells labeled with NIR680 dye. Data shown are normalized to MOS only group.

FIG. 21: HLA-Class I and II Blocking Decreased Production of Cytokines in the Co-culture of C-TIL051 and Autologous MOS.

C-TIL051: TILs generated by an embodiment of the present method.

### DETAILED DESCRIPTION

The present disclosure provides for methods of expanding tumor-infiltrating lymphocytes (TILs). The present methods do not need to use feeder cells such as PBMCs. In comparison to TILs generated/expanded using irradiated PBMCs, TILs generated/expanded using the present methods are associated with preferential expansion of CD8 TILs and sustain low surface levels of exhaustion markers like PD-1 on TILs. Specifically, TILs generated/expanded using the present method: (1) maintain a relatively higher ratio of CD8/CD4; (2) have higher percentages of Tnaive/scm and Tcm among CD4 and CD8 T cells; and (3) display reduced positivity rate of PD-1 on CD4 and CD8 T cells. The present methods can rapidly expand TILs with a high CD8/CD4 ratio, and can generate TILs with a higher ratio of CD8 cytotoxic T cells, reduced exhaustion, and enhanced memory. Accordingly, the TILs generated by the present methods are more effective for cancer immunotherapy.

Also encompassed by the present disclosure is an autologous TIL therapy that uses tumor-tissue T cells capable of recognizing tumor antigens and being expanded ex vivo while maintaining the heterogeneous repertoire of T cells, using a centralized manufacturing process.

In certain embodiments, the present cells contain autologous TILs harvested from a patient's tumor sample. Tumors may be first fragmented and cultured in high dose IL-2 to promote the egress and expansion of TILs. This initial stage of propagating TILs from tumor tissue is called pre-rapid expansion protocol (pre-REP). After this initial ex vivo culture, the TILs can be further expanded with rapid expansion protocol (REP) to achieve the therapeutic dose.

The present disclosure provides for a method of expanding tumor-infiltrating lymphocytes (TILs). The method may comprise: (a) culturing a first population of cells in a first cell culture medium to generate a second population of cells, wherein the first population of cells is obtained from a tumor sample from a patient; and (b) contacting the second population of cells with a polymeric matrix comprising anti-CD3 and anti-CD28 antibodies or fragments thereof in a second cell culture medium, to generate a third population of cells, wherein the second cell culture medium comprises about 100 IU/mL to about 8,000 IU/mL interleukin-2 (IL-2).

The method may further comprise cryopreserving the second population of cells after the first expansion (pre-REP, or step (a) of the present methods). The cryopreserved second population of cells may then be thawed and be subject to the second expansion (REP, or step (b) of the present methods, e.g., being contacted with a polymeric matrix comprising anti-CD3 and anti-CD28 antibodies or fragments thereof in a second cell culture medium). The second population of cells may be subject to the second expansion (REP, or step (b) of the present methods) immediately after thawing. The second population of cells may be subject to the second expansion (REP, or step (b) of the present methods) after being recovered for less than 2 days, less than 48 hours, less than 40 hours, less than 36 hours, less than 30 hours, less than 24 hours, less than 20 hours, less than 15 hours, less than 12 hours, less than 10 hours, less than 9 hours, less than 8 hours, less than 7 hours, less than 6 hours, less than 5 hours, less than 4 hours, less than 3 hours, less than 2 hours, or less than 1 hour.

The second population of cells (which comprises pre-REP TILs) may be cryopreserved pending patient disease progression. Following patient's progression, the pre-REP TILs are thawed and expanded according to the rapid expansion protocol (REP).

For frozen and thawed cell product, thawed cells may contact a polymeric matrix comprising anti-CD3 and anti-CD28 antibodies or fragments thereof, or may be activated, after being rested overnight. Alternatively, thawed cells may contact a polymeric matrix comprising anti-CD3 and anti-CD28 antibodies or fragments thereof, or may be activated, immediately and not rested overnight. In one embodiment, frozen cells are rapidly thawed at 37° C. in a water bath.

The volume ratio of the TransAct to the second cell culture medium (or the cell culture medium for the second expansion, or the REP cell culture medium) may range from about 1:200 to about 1:2, from about 1:180 to about 1:2, from about 1:150 to about 1:5, from about 1:120 to about 1:5, from about 1:100 to about 1:8, from about 1:80 to about 1:8, from about 1:50 to about 1:10, from about 1:40 to about 1:10, from about 1:30 to about 1:10, from about 1:20 to about 1:10, from about 1:20 to about 1:15, or about 1:17.5. In certain embodiments, the rapid expansion protocol (REP) utilizes MACS® GMP T Cell TransAct™, a colloidal polymeric nanomatrix covalently attached to humanized recombinant CD3 and CD28 agonists (e.g., antibodies). In one embodiment, 1 mL of TransAct to 17.5 mL final volume of the REP cell culture medium may be used. The REP TILs are then washed, formulated, and/or cryopreserved.

“Tumor infiltrating lymphocytes” or “TILs” may refer to a population of cells originally obtained as white blood cells that have left the bloodstream of a subject and migrated into a tumor. TILs include, but are not limited to, T cells (such as CD8⁺ cytotoxic T cells, Th1 and Th17 CD4⁺ T cells), B cells, natural killer cells, dendritic cells and macrophages. TILs may comprise (or may be) tumor-infiltrating T cells. TILs include both primary and secondary TILs. Primary TILs are those that are obtained from patient tissue samples as described herein. Secondary TILs are any TIL cell populations that have been expanded or proliferated as discussed herein, including, but not limited to, bulk TILs and expanded TILs (“REP TILs” or “post-REP TILs”). TIL cell populations can include genetically modified TILs. TILs can generally be defined either biochemically, using cell surface markers, or functionally, by their ability to infiltrate tumors and effect treatment. TILs can be generally categorized by expressing one or more of the following biomarkers: CD3, CD4, CD8, TCR α/β, CD27, CD28, CCR7, CD62L, CD45RA, CD45RO, CD95, PD-1, TIM-3, LAG-3, 4-1BB, and CD25. Additionally and alternatively, TILs can be functionally defined by their ability to infiltrate solid tumors upon reintroduction into a patient. Fisher et al., Tumor localization of adoptively transferred indium-111 labeled tumor infiltrating lymphocytes in patients with metastatic melanoma, J. Clin. Oncol., 1989, 7(2):250-61.

The term “IL-2” (or “IL2”) refers to the T cell growth factor known as interleukin-2, and includes all forms of IL-2 including human and mammalian forms, conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated by reference herein. IL-2 can encompass human, recombinant forms of IL-2 such as aldesleukin (Proleukin©), as well as the form of recombinant IL-2. Aldesleukin (des-alanyl-1, serine-125 human IL-2) is a non-glycosylated human recombinant form of IL-2. IL-2 can also encompass pegylated forms of IL-2, including the pegylated IL2 prodrug NKTR-214. Alternative forms of conjugated IL-2 suitable for use in the invention are described in U.S. Pat. Nos. 4,766,106, 5,206,344, 5,089,261 and 4,902,502, the disclosures of which are incorporated by reference herein. Formulations of IL-2 suitable for use in the invention are described in U.S. Pat. No. 6,706,289, the disclosure of which is incorporated by reference herein.

The first expansion (pre-REP) may be performed in a closed container with a first gas-permeable surface area. The second expansion (REP) may be performed in a closed container with a second gas-permeable surface area. In some embodiments, the closed container comprises (or is) a single bioreactor. In some embodiments, the closed container comprises (or is) a G-Rex® container and/or a Xuri™ cell bag. In some embodiments, the closed container is a G-Rex® device and/or Xuri™ W25 bioreactor. In some embodiments, the closed container comprises (or is) a G-Rex®10M and/or G-Rex®100M. In some embodiments, the closed container comprises (or is) a G-Rex®10M, G-Rex®100M, and/or Xuri™ W25 bioreactor. In certain embodiments, the present method (e.g., in the pre-REP and/or REP TIL culture process) involves the use of gas permeable rapid expansion (G-Rex®) devices as TIL culture vessels. In an embodiment, TILs are expanded in gas-permeable bags. In an embodiment, TILs are expanded using a cell expansion system that expands TILs in gas permeable bags, such as the Xuri™ W25 bioreactor. In an embodiment. TILs are expanded using a cell expansion system that expands TILs in gas permeable bags, such as the WAVE Bioreactor System, also known as the Xuri™ Cell Expansion System W5. In an embodiment, the cell expansion system includes a gas permeable cell bag. In an embodiment, TILs can be expanded in G-Rex® flasks.

In some embodiments, the closed system uses one container from the time the tumor fragments are obtained until the TILs are ready for administration to the patient or cryopreserving. In some embodiments when two containers are used, the first container is a closed G-Rex® container and the population of TILs is centrifuged and transferred to an infusion bag without opening the first closed G-Rex® container.

The present disclosure also provides for tumor-infiltrating lymphocytes obtained by the present methods.

Also encompassed by the present disclosure is a cell population enriched for, or expanded from, tumor-infiltrating lymphocytes, comprising one or more of the following:
(i) CD3⁺ CD8⁺ T cells at a percentage ranging from about 3% to about 88% of CD3⁺ cells,
(ii) CD3⁺ CD4⁺ T cells at a percentage ranging from about 10% to about 96% of CD3⁺ cells,
(iii) CD4⁺ T_{CM} T cells at a percentage ranging from about 50% to about 88% of CD4⁺ cells,
(iv) CD8⁺ T_{CM} T cells at a percentage ranging from about 28% to about 82% of CD8⁺ cells, (v) CD4⁺ T_{EM} T cells at a percentage ranging from about 11% to about 49% of CD4⁺ cells, and (vi) CD8⁺ T_{EM} T cells at a percentage ranging from about 11% to about 61% of CD8⁺ cells, wherein the cell population comprises no less than 70% of live cells, and wherein the cell population is generated from a tumor sample from a patient.

In certain embodiments, the cell population may comprise no less than 80% CD3 T cells in live cells.

In certain embodiments, the cell population may comprise CD4⁺ CD27⁺ T cells at a percentage ranging from about 10% to about 51% of CD4⁺ cells.

In certain embodiments, the cell population may comprise CD8⁺ CD27⁺ T cells at a percentage ranging from about 12% to about 72% of CD8⁺ cells.

In certain embodiments, the cell population may comprise CD8⁺ CD28⁺ T cells at a percentage ranging from about 34% to about 95% of CD8⁺ cells.

In certain embodiments, the cell population may comprise CD4⁺CD28⁺ T cells at a percentage ranging from about 82% to about 100% of CD4⁺ cells.

In certain embodiments, the cell population may comprise CD4⁺ 4-1BB⁺ T cells at a percentage ranging from about 0.2% to about 5.8% of CD4⁺ cells.

In certain embodiments, the cell population may comprise CD8⁺ 4-1BB⁺ T cells at a percentage ranging from about 0.2% to about 11.6% of CD8⁺ cells.

In certain embodiments, the cell population may comprise CD4⁺ LAG3⁺ T cells at a percentage ranging from about 0.2% to about 19.5% of CD4⁺ cells.

In certain embodiments, the cell population may comprise CD8⁺ LAG3⁺ T cells at a percentage ranging from about 6% to about 51.2% of CD8⁺ cells.

In certain embodiments, the cell population may comprise CD4⁺ PD1⁺ T cells at a percentage ranging from about 0.9% to about 31% of CD4⁺ cells.

In certain embodiments, the cell population may comprise CD8⁺ PD1⁺ T cells at a percentage ranging from about 1% to about 18% of CD8⁺ cells.

In certain embodiments, the cell population may comprise no greater than 10% CD56-NK cells.

The present disclosure provides for a cell population, generated by a method of expanding tumor-infiltrating lymphocytes (TILs). The method may comprise: (a) culturing a first population of cells in a first cell culture medium to generate a second population of cells, wherein the first population of cells is obtained from a tumor sample from a patient; and (b) contacting the second population of cells with a polymeric matrix comprising anti-CD3 and anti-CD28 antibodies or fragments thereof in a second cell culture medium, wherein the second cell culture medium comprises about 100 IU/mL to about 8,000 IU/mL interleukin-2 (IL-2).

The present disclosure provides for a method of expanding a cell population enriched for tumor-infiltrating lymphocytes. The method may comprise: (a) culturing cells obtained from a tumor sample from a patient; (b) treating the cultured cells to generate a cell population enriched for tumor-infiltrating lymphocytes, the cell population comprising one or more of the following:
(i) CD3⁺ CD8⁺ T cells at a percentage ranging from about 3% to about 88% of CD3⁺ cells,
(ii) CD3⁺ CD4⁺ T cells at a percentage ranging from about 10% to about 96% of CD3⁺ cells,
(iii) CD4⁺ CD45RA⁺CD62L+ central memory T cells at a percentage ranging from about 50% to about 88% of CD4⁺ cells,
(iv) CD8⁺CD45RA⁺CD62L⁺ central memory T cells at a percentage ranging from about 28% to about 82% of CD8+ cells,
(v) CD4⁺ CD45RA⁻CD62L⁻ effector memory T cells at a percentage ranging from about 11% to about 49% of CD4⁺ cells, and
(vi) CD8⁺ T CD45RA⁻CD62L⁻ effector memory T cells at a percentage ranging from about 11% to about 61% of CD8⁺ cells.

The present disclosure also provides for a cell population enriched for tumor-infiltrating lymphocytes obtained by the present methods.

The present methods can use any suitable cell culture media. In certain embodiments, the present methods use one or more types of cell culture media. Cell culture media that may be used include, but are not limited to, AIM-V medium (L-glutamine, streptomycin sulfate at 50 pLg/ml, and gentamicin sulfate at 10 μg/ml) (Thermo Fisher), and RPMI 1640 medium.

A concentration of IL-2 (in the first culture medium or the second culture medium) can be about 1000 IU/mL to about 10000 IU/mL, about 2000 IU/mL to about 10000 IU/mL, about 3000 IU/mL to about 10000 IU/mL, about 4000 IU/mL to about 10000 IU/mL, about 5000 IU/mL to about 10000 IU/mL, about 2000 IU/mL to about 8000 IU/mL, about 3000 IU/mL to about 7000 IU/mL, or about 4000 IU/mL to about 6000 IU/mL. A concentration of IL-2 (in the first culture medium or the second culture medium) can be about 6000 IU/mL. A concentration of IL-2 (in the first culture medium or the second culture medium) can also be about 2000 IU/mL, 3000 IU/mL, 4000 IU/mL, 5000 IU/mL, 6000 IU/mL, 7000 IU/mL, 8000 IU/mL, 9000 IU/mL, or up to about 10000 IU/mL.

Once TILs are expanded, they can be subject to in vitro assays to determine their properties and/or functions such as tumor reactivity. For example, TILs can be evaluated by FACS for CD3, CD4, CD8, and CD58 expression. TILs can also be subjected to co-culture, cytotoxicity, ELISA, or ELISPOT assays.

The tumor sample may be obtained from any mammal. The present cells or pharmaceutical compositions may be used to treat any mammal. The mammal may be a human or a non-human primate. Mammals also include, but are not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). It is preferred that the manunals are non-human primates, e.g., of the order Primates, Ceboids, or Simoids (monkeys) or of the order Anthropoids (humans and apes). In some embodiments, the mammal may be a mammal of the order Rodentia, such as mice and hamsters.

### Obtaining Tumor Samples

In general, TILs are initially obtained from a patient tumor sample (the first population of cells) and then expanded into a larger population for further manipulation as described herein, optionally cryopreserved.

TILs can be expanded from a tumor sample, or a tissue or organ afflicted with a cancer. In some embodiments, the TILs are obtained from tumor fragments. In some embodiments, a tumor (sample) can be trimmed from non-cancerous tissue or necrotic areas. A patient tumor sample may be obtained using methods known in the art, e.g., via surgical resection, needle biopsy or other means for obtaining a sample that contains a mixture of tumor and TIL cells. Once obtained, a tumor sample may be fragmented (e.g., using sharp dissection) into small pieces. The first population of cells may be one or multiple tumor fragments from a patient (or more than one patient). The first population of cells may be obtained from one or multiple tumor fragments from a tumor resected from a patient. The processes for disrupting a tumor may include mechanical fragmentation methods such as crushing, slicing, dividing, and morcellating tumor tissue as well as any other method for disrupting the physical structure of tumor tissue.

In some embodiments, the tumor fragments are between about 1 mm³ and 50 mm³, between about 1 mm³ and 45 mm³, between about 1 mm³ and 40 mm³, between about 1 mm³ and 35 mm³, between about 1 mm³ and 30 mm³, between about 1 mm³ and 25 mm³, between about 1 mm³ and 20 mmn³, between about 1 mm³ and 15 mm³, between about 1 mm³ and 10 mm³, between about 1 mm³ and 8 mm³, between about 2 mm³ and 3 mm³, about 1 mm³, about 2 mm³, about 3 mm³, about 4 mm³, about 5 mm³, about 6 mm³, about 7 mm³, about 8 mm³, about 9 mm³, about 10 mm³, about 12 mm³, about 15 mm³, about 18 mm³, about 20 mm³, about 25 mm³, or about 30 mm³. In some embodiments, the multiple fragments comprise about 4 to about 50 fragments, wherein each fragment has a volume of about 8 mm³ to about 27 mm³. In some embodiments, the multiple fragments comprise about 30 to about 60 fragments with a total volume of about 1300 mm³ to about 1500 mm³. In some embodiments, the multiple fragments comprise about 50 fragments with a total volume of about 1350 mm³. In some embodiments, the multiple fragments comprise about 50 fragments with a total mass of about 1 gram to about 1.5 grams.

In some embodiments, a tumor can be fragmented to about 2-3 mm in each dimension. In some cases, a tumor can be fragmented from about 0.5 mm to about 5 mm, from about 1 mm to about 2 mm, from about 2 mm to about 3 mm, from about 3 mm to about 4 mm, or from about 4 mm to about 5 mm in each dimension.

The TILs may be cultured from these tumor fragments using enzymatic tumor digests. Such tumor digests may be produced by incubation in enzymatic media (e.g., DNase and collagenase) followed by mechanical dissociation (e.g., using a tissue dissociator). In some embodiments, fragmentation includes physical fragmentation (e.g., dissection) and/or digestion.

The method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

In certain embodiments, the tumor sample is from a solid tumor, including primary tumors, invasive tumors, or metastatic tumors. Solid tumors may be benign or malignant. The term “solid tumor cancer” refers to malignant, neoplastic, or cancerous solid tumors. The solid tumor may comprise a sarcoma, hepatocellular carcinoma, glioma, head-neck cancer, bone cancer, brain cancer, breast cancer, cancer of the anus, cancer of the anal canal, cancer of the anorectum, cancer of the eye, cancer of the intrahepatic bile duct, cancer of the joints, cancer of the neck, cancer of the gallbladder, cancer of the pleura, cancer of the nose, cancer of the nasal cavity, cancer of the middle ear, cancer of the oral cavity, cancer of the vulva, colon cancer, esophageal cancer, cervical cancer, gastrointestinal cancer, hypopharynx cancer, larynx cancer, liver cancer, lung cancer, malignant mesothelioma, melanoma, nasopharynx cancer, ovarian cancer, pancreatic cancer, peritoneum cancer, omentum cancer, mesentery cancer, pharynx cancer, prostate cancer, rectal cancer, renal cancer, small intestine cancer, soft tissue cancer, stomach cancer, testicular cancer, thyroid cancer, ureter cancer, urinary bladder cancer, or combinations thereof. In some embodiments, the cancer is selected from cervical cancer, head and neck cancer (including, for example, head and neck squamous cell carcinoma (HNSCC)) glioblastoma, ovarian cancer, sarcoma, pancreatic cancer, bladder cancer, breast cancer, and non-small cell lung carcinoma.

The tumor sample may also be from a liquid tumor, such as a tumor obtained from a hematological malignancy.

TILs can be expanded from a tumor sample from a donor of any stage of development including, but not limited to, fetal, neonatal, young and adult.

Tumor fragments can then be cultured in vitro utilizing media and a cellular stimulating agent such as a cytokine. In some cases, IL-2 can be utilized to expand TILs from a tumor fragment.

### First Expansion (Pre-REP)

Pre-REP TILs expanded by the present methods can generate REP TILs without markedly reduced CD8/CD4 ratio. The present pre-REP TILs can be expanded while maintaining a low expression level of exhaustion marker PD-1 in REP TILs.

In the first expansion (pre-REP, step (a) of the method), the first cell culture medium may comprise about 1,000 IU/mL to about 10,000 IU/mL IL-2, about 1,000 IU/mL to about 9,000 IU/mL IL-2, about 1,000 IU/mL to about 8,000 IU/mL IL-2, about 2,000 IU/mL to about 8,000 IU/mL IL-2, about 2,000 IU/mL to about 7,000 IU/mL IL-2, about 2,000 IU/mL to about 6,000 IU/mL IL-2, about 2,000 IU/mL to about 5,000 IU/mL IL-2, about 2,000 IU/mL to about 4,000 IU/mL IL-2, about 2,000 IU/mL to about 3,000 IU/mL IL-2, about 3,000 IU/mL to about 8,000 IU/mL IL-2, about 3,000 IU/mL to about 7,000 IU/mL IL-2, about 3,000 IU/mL to about 6,000 IU/mL IL-2, about 3,000 IU/mL to about 5,000 IU/mL IL-2, about 4,000 IU/mL to about 8,000 IU/mL IL-2, about 2,000 IU/mL IL-2, about 3,000 IU/mL IL-2, about 4,000 IU/mL IL-2, about 5,000 IU/mL IL-2, about 6,000 IU/mL IL-2, about 7,000 IU/mL IL-2, about 8,000 IU/mL IL-2, about 9,000 IU/mL IL-2, or about 10,000 IU/mL IL-2. In certain embodiments, the first cell culture medium may comprise about 3,000 IU/mL IL-2. In certain embodiments, the first cell culture medium may comprise about 5,000 IU/mL IL-2. In certain embodiments, the first cell culture medium may comprise about 6,000 IU/mL IL-2. In certain embodiments, the first cell culture medium may comprise about 7,000 IU/mL IL-2. In certain embodiments, the first cell culture medium may comprise about 8,000 IU/mL IL-2.

In the first expansion (pre-REP, step (a) of the method), the first population of cells may be cultured (the first expansion may last) for about 3 days to about 60 days, about 5 days to about 50 days, about 7 days to about 40 days, about 10 days to about 40 days, about 10 days to about 30 days, about 10 days to about 20 days, about 10 days to about 18 days, about 10 days to about 17 days, about 10 days to about 16 days, about 12 days to about 15 days, about 12 days to about 14 days, about 13 days to about 14 days, about 10 days, about 11 days, about 12 days, about 13 days, about 14 days, about 15 days, about 16 days, about 17 days, about 18 days, about 19 days, or about 20 days. In certain embodiments, the first population of cells is cultured for about 14 days. In certain embodiments, the first expansion lasts for about 14 days.

In the first expansion (pre-REP, step (a) of the method), the cell culture medium may be added (or changed) once, twice, three times, or four times, when the first population of cells has been cultured for about 2 or 3 days (on or around day 3, when day 0 is the day when the culture of the first population of cells starts), for about 3 or 4 days (on or around day 4), for about 4 or 5 days (on or around day 5), for about 5 or 6 days (on or around day 6), for about 6 or 7 days (on or around day 7), for about 7 or 8 days (on or around day 8), for about 8 or 9 days (on or around day 9), for about 9 or 10 days (on or around day 10), for about 10 or 11 days (on or around day 11), for about 11 or 12 days (on or around day 12), for about 12 or 13 days (on or around day 13), or for about 13 or 14 days (on or around day 14). In certain embodiments, in the first expansion (pre-REP, step (a) of the method), the cell culture medium is added once on or around day 9 (when the first population of cells has been cultured for about 8 or 9 days). In certain embodiments, in the first expansion (pre-REP, step (a) of the method), the cell culture medium is added once on or around day 10 (when the first population of cells has been cultured for about 9 or 10 days). In certain embodiments, in the first expansion (pre-REP, step (a) of the method), the cell culture medium is added once on or around day 11 (when the first population of cells has been cultured for about 10 or 11 days).

In certain embodiments, in the first expansion (pre-REP, step (a) of the method), the volume of the added cell culture medium is about 20%, about 30%, about 40%, about 50%, about 60%, about 70%, about 80%, about 90%, about 100%, about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.2-fold, about 2.5-fold, about 2.7-fold, or about 3-fold, of the volume of the cell culture (medium) in the cell culture container.

The initial culture volume for the first expansion (pre-REP, step (a) of the method) may range from about 5% to about 100%, from about 10% to about 80%, from about 10% to about 60%, from about 10% to about 50%, from about 10% to about 40%, from about 10% to about 30%, from about 20% to about 80%, from about 20% to about 60%, from about 20% to about 50%, from about 20% to about 40%, from about 20% to about 30%, from about 25% to about 80%, from about 25% to about 60%, from about 25% to about 50%, from about 25% to about 40%, from about 25% to about 30%, about 5%, about 10%, about 15%, about 20%, about 25%, about 30%, about 35%, about 40%, about 45%, about 50%, about 55%, or about 60%, of the capacity of the cell culture container/system. In certain embodiments, the initial culture volume for the first expansion (pre-REP, step (a) of the method) is about 10%, about 15%, about 20%, about 25%, about 30%, about 35%, or about 40%, of the capacity of the cell culture container/system. In certain embodiments, 10 mL, 15 mL, 20 mL, 25 mL, 30 mL, 35 mL, or 40 mL of initial culture volume is used for the G-Rex®10M device (bottom surface area 10 cm², 100 mL volume capacity). In certain embodiments, 100 mL, 150 mL, 200 mL, 250 mL, 300 mL, 350 mL, or 400 mL of initial culture volume is used for the G-Rex®100M device (bottom surface area of 100 cm², 1000 mL volume capacity).

The first expansion (pre-REP) may result in a population of cells or TILs ranging from about 1×10⁷ to about 1×10⁹ TILs or cells, from about 2×10⁷ to about 9×10⁸ TILs or cells, from about 3×10⁷ to about 8×10⁸ TILs or cells, from about 4×10⁷ to about 6×10⁸ TILs or cells, from about 5×10⁷ to about 5×10⁷ TILs or cells, from about 6×10⁷ to about 4×10⁸ TILs or cells, from about 8×10⁷ to about 3×10⁸ TILs or cells, from about 9×10⁷ to about 2×10⁸ TILs or cells, from about 9×10⁷ to about 1×10⁸ TILs or cells, or about 1×10⁸ TILs or cells.

### Second Expansion (REP)

The second expansion is generally referred to as a rapid expansion process (REP). The second expansion of the present method may or may not use feeder cells. In one embodiment, the present method, or the second expansion of the present method, is a feeder cell-free process.

In the second expansion (REP, step (b) of the method), the second cell culture medium may comprise about 50 IU/mL to about 10,000 IU/mL IL-2, about 100 IU/mL to about 8,000 IU/mL IL-2, about 200 IU/mL to about 6,000 IU/mL IL-2, about 500 IU/mL to about 6,000 IU/mL IL-2, about 500 IU/mL to about 4,000 IU/mL IL-2, about 500 IU/mL to about 3,000 IU/mL IL-2, about 800 IU/mL to about 5,000 IU/mL IL-2, about 800 IU/mL to about 4,000 IU/mL IL-2, about 1,000 IU/mL to about 6,000 IU/mL IL-2, or about 1,000 IU/mL to about 4,000 IU/mL IL-2. In one embodiment, the second cell culture medium comprises about 3,000 IU/mL IL-2.

In the second expansion (REP, step (b) of the method), the contacting may be for about 3 days to about 17 days, for about 3 days to about 60 days, about 5 days to about 50 days, about 7 days to about 40 days, about 10 days to about 40 days, about 10 days to about 30 days, about 10 days to about 20 days, about 10 days to about 14 days.

In some embodiments, the second expansion (REP, step (b) of the method) can proceed for 7 days, 8 days, 9 days, 10 days, 11 days, 12 days, 13 days, or 14 days. In some embodiments, the second TIL expansion can proceed for about 7 days to about 14 days. In some embodiments, the second TIL expansion can proceed for about 8 days to about 14 days. In some embodiments, the second TIL expansion can proceed for about 9 days to about 14 days. In some embodiments, the second TIL expansion can proceed for about 10 days to about 14 days. In some embodiments, the second TIL expansion can proceed for about 11 days to about 14 days. In some embodiments, the second TIL expansion can proceed for about 12 days to about 14 days. In some embodiments, the second TIL expansion can proceed for about 13 days to about 14 days. In some embodiments, the second TIL expansion can proceed for about 14 days.

In the second expansion (REP, step (b) of the method), the contacting may occur, e.g., in vitro in any container capable of holding cells, preferably in a sterile environment. Such containers may be, e.g., culture flasks, culture bags, bioreactors or any device that can be used to grow cells, including in a closed cell culture system or a closed container. The closed system or container may provide a gas-permeable surface area. In an embodiment, the second expansion (REP, step (b) of the method) may be performed using T-175 flasks, gas permeable bags, and/or gas permeable culture system (e.g., G-Rex®). In some embodiments, the second expansion (REP, step (b) of the method) is performed in a closed system or a closed system bioreactor. In some embodiments, the closed system bioreactor is a single bioreactor. In an embodiment, the second expansion may be performed in G-Rex®10M and/or G-Rex®100M.

In certain embodiments, the third population of cells is at least 100-fold greater in number than the second population of cells, for example, about 100-fold to about 2000-fold greater in number, about 100-fold to about 1800-fold greater in number, about 100-fold to about 1500-fold greater in number, about 100-fold to about 1200-fold greater in number, about 100-fold to about 1000-fold greater in number, about 100-fold to about 800-fold greater in number, about 100-fold to about 600-fold greater in number, or about 100-fold to about 500-fold greater in number, than the second population of cells.

The third population of cells may be a therapeutic population of TILs. In some embodiments, the therapeutic population of TILs comprises sufficient TILs for a therapeutically effective dosage of the TILs.

In some embodiments, the third population of cells comprises an increased subpopulation of effector T cells and/or central memory T cells relative to the second population of TILs. In some embodiments, the third population of TILs preserves the TCR repertoire of the second population of TILs to a greater extent.

### Polymeric Matrix Comprising Anti-CD3 and Anti-CD28 Antibodies or Fragments

In certain embodiments, the present methods use a polymeric matrix comprising anti-CD3 and anti-CD28 antibodies or fragments thereof to generate, expand, activate, and/or enrich for, TILs.

The flexible matrix may comprise (or consist essentially of, or consist of) collagen, protein, peptide, polysaccharide, glycosaminoglycan, and/or extracellular matrix compositions. A polysaccharide may include, for example, cellulose, agarose, dextran, chitosan, hyaluronic acid, or alginate. Other polymers may include polyesters, polyethers, polyanhydrides, polyalkylcyanoacrylates, polyacrylamides, polyorthoesters, polyphosphazenes, polyvinylacetates, block copolymers, polypropylene, polytetrafluorethylene (PTFE), or polyurethanes. The polymer may be lactic acid or a copolymer. A copolymer may comprise lactic acid and/or glycolic acid (PLGA). The polymeric matrix may comprise (or consist essentially of, or consist of) polymeric dextran material (or a polymer of dextran).

The polymeric matrix may have an average molecular weight of 40,000 daltons.

The polymeric matrix may or may not comprise magnetic, paramagnetic, superparamagnetic nano-crystals, or fluorescent dyes (e.g., embedded into the polymeric matrix).

The polymeric matrix may have sizes smaller than 1 m, smaller than 500 nm, or smaller than 200 nm. The polymeric matrix may have sizes ranging from about 1 nm to about 500 nm, or from about 10 nm to about 200 nm.

The anti-CD3 antibodies or fragments thereof and the anti-CD28 antibodies or fragments thereof may be attached to the same polymeric matrix, or attached to separate polymeric matrices. The anti-CD3 antibodies or fragments thereof and anti-CD28 antibodies or fragments thereof may be attached or coupled to the polymeric matrix by a variety of methods known in the art. The attachment may be covalent or noncovalent, electrostatic, or hydrophobic. The attachment may be accomplished by a variety of attachment means, including, for example, chemical, mechanical, enzymatic, or other suitable means. The antibody or a fragment thereof first may be attached to the matrix directly or indirectly. For example, the antibody or a fragment thereof first may be attached to the matrix through the avidin (or streptavidin) and biotin system. The antibody or a fragment thereof may be attached to the matrix indirectly, e.g., via an anti-isotype antibody. Another example includes using protein A or protein G, or other non-specific antibody binding molecules, attached to matrices to bind an antibody or a fragment thereof. Alternatively, the antibody or a fragment thereof may be attached to the matrix by chemical means, such as cross-linking to the matrix.

The anti-CD3 and/or anti-CD28 antibodies may be polyclonal and monoclonal antibodies, chimeric antibodies, haptens and antibody fragments, and molecules which are antibody equivalents in that they specifically bind to an epitope on the antigen. The term “antibody” includes polyclonal and monoclonal antibodies of any isotype (IgA, IgG, IgE, IgD, IgM), or an antigen-binding portion thereof, including, but not limited to, F(ab) and Fv fragments such as scFv, single chain antibodies, chimeric antibodies, humanized antibodies, and a Fab expression library. In certain embodiments, the anti-CD3 and/or anti-CD28 antibodies may be monoclonal antibodies. Anti-CD3 antibodies include OKT-3, also known as muromonab. Anti-CD3 antibodies also include the UHCT1 clone, also known as T3 and CD3E. Other anti-CD3 antibodies include, for example, otelixizumab, teplizumab, and visilizumab.

The ratio of the anti-CD3 antibodies or fragments thereof to anti-CD28 antibodies or fragments thereof (attached to the same polymeric matrix, or attached to separate polymeric matrices), may range from about 100:1 to about 1:100, from about 10:1 and about 1:10, or from about 2:1 and about 1:2. The anti-CD3 antibodies or fragments thereof and/or anti-CD28 antibodies or fragments thereof may be attached to the same or separate matrices at high density, with more than 25 μg per mg matrix, or with more than 50 μg per mg matrix.

In one embodiment, the polymeric matrix is TransAct™. U.S. Pat. No. 10,513,687.

In certain embodiment, the ratio of the polymeric matrices to cells may be larger than 100:1, larger than 500:1, or larger than 1000:1.

### Pharmaceutical Compositions

The present disclosure provides for a pharmaceutical composition comprising the present cells/TILs or cell populations. The present pharmaceutical composition may comprise the present cells or cell populations and a pharmaceutically acceptable carrier or excipient. Pharmaceutically acceptable carriers or pharmaceutically acceptable excipients may include suitable solvents, dispersion media, coatings, antibacterial and antifungal agents, isotonic and absorption delaying agents, and inert ingredients. Additional active pharmaceutical ingredients, such as other drugs, can also be incorporated into the described compositions and methods. A suitable pharmaceutically acceptable carrier for the cells for injection may include any isotonic carrier such as, for example, normal saline (about 0.9% w/v of NaCl in water, about 300 mOsm/L NaCl in water, or about 9.0 g NaCl per liter of water), or about 5% dextrose in water. In an embodiment, the pharmaceutically acceptable carrier is supplemented with human serum albumin. Compositions of the present disclosure may be formulated for intravenous administration. In an embodiment, the pharmaceutical composition is a suspension of TILs or cells in a sterile buffer.

The present cells or pharmaceutical compositions may be used in a method for treating diseases, such as hyperproliferative disorders. They may also be used in treating other disorders. In some embodiments, the hyperproliferative disorder is cancer.

The present cells or cell populations can be used in methods of treating or preventing cancer. In this regard, the disclosure provides a method of treating or preventing cancer in a mammal, comprising administering to the mammal the present pharmaceutical compositions, cells, or cell populations in an amount effective to treat or prevent cancer in the mammal. Another embodiment of the invention provides a method of treating or preventing cancer in a mammal, comprising administering present cells or cell populations to a mammal in an amount effective to treat or prevent cancer in the mammal.

Also encompassed by the present disclosure is a method of treating a subject (e.g., a mammal) or patient with cancer. The method may comprise administering to the subject or patient the present TILs, and/or the present cell population. The cells/TILs may be administered to a patient as a pharmaceutical composition.

The present cells or pharmaceutical compositions may be administered by any suitable routes, including intranasal and transdermal routes, intra-arterial routes, intravenously, intraperitoneally, parenterally, intramuscularly, subcutaneously, topically, by transplantation, or by inhalation. The present cells or pharmaceutical compositions may be administered by injection or infusion. In some embodiments, the present cells or pharmaceutical compositions are administered via intra-arterial or intravenous administration (e.g., infusion). Other suitable routes of administration include intraperitoneal, intrathecal, and intralymphatic.

Any suitable dose of TILs can be administered. In some embodiments, from about 2×10¹⁰ to about 15×10¹⁰ TILs or cells, about 1×10¹⁰ to about 5×10¹⁰ of TILs or cells, about 3×10¹⁰ to about 12×10¹⁰ TILs or cells, about 4×10¹⁰ to about 10×10¹⁰ TILs or cells, about 5×10¹⁰ to about 8×10¹⁰ TILs or cells, about 6×10¹⁰ to about 8×10¹⁰ TILs or cells, about 7×10¹⁰ to about 8×10¹⁰ TILs or cells, about 8×10¹⁰ TILs or cells, or about 7×10¹⁰ TILs or cells, are administered. In some embodiments, the therapeutically effective dosage is about 2×10¹⁰ to about 15×10¹⁰ of the TILs or cells. In some embodiments, the therapeutically effective dosage is about 1×10¹⁰ to about 5×10¹⁰ TILs or cells. In some embodiments, the therapeutically effective dosage is about 3×10¹⁰ to about 12×10¹⁰ TILs or cells. In some embodiments, the therapeutically effective dosage is about 4×10¹⁰ to about 10×10¹⁰ TILs or cells. In some embodiments, the therapeutically effective dosage is about 5×10¹0 to about 8×10¹⁰ TILs or cells. In some embodiments, the therapeutically effective dosage is about 6×10¹⁰ to about 8×10¹⁰ TILs or cells. In some embodiments, the therapeutically effective dosage is about 7×10¹⁰ to about 8×10¹⁰ TILs or cells. In some embodiments, an effective dosage of TILs is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³ cells/TILs. In some embodiments, an effective dosage of TILs is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, to 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³ cells/TILs. In certain embodiments, about 1×10⁹ to about 1×10¹¹ cells, or about 5×10⁹ to about 9×10¹⁰ cells, are administered to the patient. The number of cells/TILs may be about 10×10⁶ to about 10×10¹¹ cells per administration (e.g., infusion), about 10×10⁹ cells to about 10×10¹¹ cells per administration (e.g., infusion), or 10×10⁷ to about 10×10⁹ cells per administration (e.g., infusion). A pharmaceutical composition comprising the TILs may be administered at a dosage of 10⁴ to 10¹¹ cells/kg body weight (e.g., 10⁵ to 10⁶, 10⁵ to 10¹⁰, 10⁵ to 10¹¹, 10⁶ to 10¹⁰, 10⁶ to 10¹¹, 10⁷ to 10¹¹, 10⁷ to 10¹⁰, 10⁸ to 10¹¹, 10⁸ to 10¹⁰, 10⁹ to 10¹⁰, or 10⁹ to 10¹⁰ cells/kg body weight), including all integer values within those ranges.

The TILs provided in the present pharmaceutical compositions may be effective over a wide dosage range. The exact dosage may depend upon the route of administration, the rate of administration, the severity of the disorder or condition, the gender and age of the subject to be treated, and the body weight of the subject to be treated. The clinically established dosages of the TILs may also be used if appropriate.

The present cells or pharmaceutical compositions may be administered in a single dose or in multiple doses. Such administration may be by injection, e.g., intravenous injection. In some embodiments, the present cells or pharmaceutical compositions are administered as a single intra-arterial or intravenous infusion. Dosing may be once, twice, three times, four times, five times, six times, or more than six times per year. Dosing may be once a month, once every two weeks, once a week, or once every other day. Administration of TILs may continue as long as necessary.

For purposes of the inventive methods, wherein populations of cells are administered, the cells can be cells that are allogeneic or autologous to the mammal. In one embodiment, the cells are autologous to the mammal.

The cancer treated by the present cells or pharmaceutical compositions may be melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), renal cancer, renal cell carcinoma, colorectal cancer, or other types of cancer. The present cells or pharmaceutical compositions may be used to treat relapsed or refractory non-small cell lung cancer (NSCLC). The cancer can be any cancer, including any of sarcomas (e.g., synovial sarcoma, osteogenic sarcoma, leiomyosarcoma uteri, and alveolar rhabdomyosarcoma), lymphomas (e.g., Hodgkin lymphoma and non-Hodgkin lymphoma), hepatocellular carcinoma, glioma, head-neck cancer, acute lymphocytic cancer, acute myeloid leukemia, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer (e.g., colon carcinoma), esophageal cancer, cervical cancer, gastrointestinal cancer (e.g., gastrointestinal carcinoid tumor), hypopharynx cancer, larynx cancer, liver cancer, lung cancer, malignant mesothelioma, melanoma, multiple myeloma, nasopharynx cancer, ovarian cancer, pancreatic cancer, peritoneum, omentum, and mesentery cancer, pharynx cancer, prostate cancer, rectal cancer, renal cancer, small intestine cancer, soft tissue cancer, stomach cancer, testicular cancer, thyroid cancer, ureter cancer, and urinary bladder cancer.

In some embodiments, the hyperproliferative disorder is a solid tumor cancer. In some embodiments, the solid tumor cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), renal cancer, and renal cell carcinoma. In some embodiments, the hyperproliferative disorder is a hematological malignancy. In some embodiments, the solid tumor cancer is chronic lymphocytic leukemia, acute lymphoblastic leukemia, diffuse large B cell lymphoma, non-Hodgkin's lymphoma, Hodgkin's lymphoma, follicular lymphoma, and mantle cell lymphoma.

In an embodiment, the method of treating a cancer in a patient further comprises pre-treating the patient with non-myeloablative chemotherapy prior to administration of the present cells or pharmaceutical compositions. In an embodiment, the non-myeloablative chemotherapy comprises administering cyclophosphamide and/or fludarabine prior to TIL infusion. In an embodiment, after non-myeloablative chemotherapy and TIL infusion, the patient receives an intravenous infusion of IL-2.

The term “about” in reference to a numeric value refers to ±10% of the stated numeric value. In other words, the numeric value can be in a range of 90% of the stated value to 110% of the stated value.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: TIL Pre-REP Process Development and Pre-REP TIL Characterization

In the pre-rapid expansion protocol (pre-REP), autologous tumor infiltrating lymphocytes (TILs) were harvested from a patient's NSCLC tumor sample. Tumors were first fragmented and cultured in high dose IL-2 to promote the egress and expansion of TILs. After this initial ex vivo culture, the TILs can be further expanded with rapid expansion protocol (REP) to achieve the therapeutic dose. We have optimized the pre-REP TIL culture process that can be transferred to GMP production.

The initial T cell material (pre-REP TILs) may be cryopreserved pending patient disease progression after standard of care therapy. Following patient's progression, the pre-REP TILs can be thawed and expanded according to the rapid expansion protocol (REP).

Our optimized pre-REP TIL culture process involves the use of gas permeable rapid expansion (G-Rex®) devices as TIL culture vessels and is conducted with a shorter culture time and media exchange frequency to support a robust GMP manufacturing process that generates pre-REP TILs with a high cell yield and favorable T cell phenotype suitable for further manufacture. Media may be added on or around, day 8, day 9, day 10, and/or day 11 (day 0 being the day when the cell culture (the first expansion, or pre-REP) starts), and pre-REP TILs may be harvested on or around day 12, day 13, day 14, day 15, day 16, or day 17. With the optimized protocol, IL-2 and glucose levels remain sufficient, while the growth inhibitory metabolite lactic acid is at low levels that are not detrimental to TIL expansion. We demonstrated after optimization that the average number of TILs produced per tumor fragment is more than 4 times higher than the number of TILs produced before optimization (16.29×10⁶±10.58×10⁶ vs 3.10×10⁶±3.52×10⁶, respectively), which represented overall superior pre-REP TIL yields. The pre-REP TILs display an earlier differentiation state, with a higher percentage of CD27⁺ cells, a majority T central memory (66.28% 12.87% among CD4⁺ compartment and 59.04%±13.47% among CD8⁺ compartment; n=8), and very low proportions of effector cells (0.26%±0.19% and 4.27% 3.85% among CD4¹ and CD8⁺ cells, respectively; n=8). In addition, the percentages of T_{N}/T_{SCM} subsets were also high in the harvested products indicating a more favorable TIL characteristic¹.

In one embodiment, the rapid expansion protocol (REP) utilizes MACS® GMP T Cell TransAct™, a colloidal polymeric nanomatrix covalently attached to humanized recombinant CD3 and CD28 agonists (e.g., antibodies). The REP TIL is then washed, formulated, and/or cryopreserved.

During pre-REP, fragmented melanoma tumors grown in G-Rex® devices were previously shown to produce an average of 7.51×10⁶ TILs per tumor fragment with the average of 14-18 days culture². Our pre-REP manufacturing target cell number was set at 6×10⁷ CD3⁺ T cells based on an estimated REP expansion of 400 times and a TIL target dose of minimum 1×10⁺ cells based on previous publication³. Appropriate conditions that favor the growth and preservation of early differentiation phenotypes of pre-REP TILs will generate a high-quality intermediate, which is expected to facilitate the manufacturing of final REP TIL product with high therapeutic potential^{4,5}. This study sought to optimize key culture conditions to generate high yield products of pre-REP TILs with early differentiation phenotypes and shortened culture time.

### Methods

### Tumor Collection

Non-small cell lung cancer tumor specimens were collected. The NSCLC specimens were de-identified and each donor was assigned with one identification number. Briefly, resected tumor samples were submerged in the tissue preservation solution at 4° C. and shipped within 24 hours of surgery. Immediately upon reception, tumor samples were transferred for preparation of TILs.

### Tumor Fragmentation and Seeding

Only one tumor was processed at a time and a new set of materials and devices was used for different tumors.

Tumor wash media was added to the wells of a 6-well plate for washing. The tumor wash media was added to a petri-dish for tumor fragmentation. Tumor was transferred to the petri-dish using forceps. Tumor was dissected into fragments of ˜2-3 mm in each dimension. Areas of necrotic, hemorrhagic, and fatty tissue were removed during fragmentation. Each tumor fragment was washed to remove erythrocytes. The process was repeated until the whole tumor section was dissected. The number of tumor fragments was recorded and fragments from same donor were combined for culture in the same culture device.

For the use of G-Rex®10M devices, 1, 2, 3, 4, 5, 6, 7 or 8 tumor fragments of the same donor were seeded into one G-Rex®10M device. For the use of G-Rex®100M devices, up to 100 tumor fragments of the same donor were seeded into one G-Rex®10M device.

### Growing of Pre-REP TILs

Pre-REP TILs were cultured in pre-REP basal media supplemented with 300 IU/mL, 1000 IU/mL, 3000 IU/mL, or 6000 IU/mL IL-2. Basal media include RPMI-1640, human AB serum, HEPES (4-(2-hydroxyethyl)-1-piperazineethanesulfonic acid), β-ME (2-Mercaptoethanol), GlutaMax and Gentamicin.

On day 0 after tumor fragmentation, tumor fragments were seeded in G-Rex®10M culture devices. For a small-scale pre-REP, 1-8 fragments were added to a G-Rex®10M device with initial media volume of 10 mL, 20 mL, 25 mL, 30 mL, 50 mL or 100 mL.

### Cell Count

Cell concentration, viability, and size were measured using the NucleoCounter® NC-200™ system.

### Collection of TIL Culture Medium Samples

If spent media was removed before cell counting, 500 μL of the spent media was saved in a −20° C. freezer for later analysis. Alternatively, if no cell counting was performed, a small volume (˜500 μL each time) of pre-REP culture medium was collected from near the surface of the media. Media were sampled from the G-Rex®10M devices or G-Rex®100M devices at the indicated time points (e.g., Day 0, 3, 6, 7, 10 and 14) during the pre-REP stage.

### Quantification of Glucose and Lactate Concentrations in Cultures

The glucose and lactate concentrations in culture supernatants were measured by the Glucose-Glo Assay and Lactate-Glo Assay (Promega).

### IL-2 Concentration Quantification by ELISA

The IL-2 ELISA kits from Thermo Fisher Scientific, Inc. were used to quantify IL-2 protein concentrations. Samples were measured in duplicate or triplicate wells, and data are presented as Mean±Standard Deviation (S.D.). The protein concentrations (pg/mL) were converted to IU/mL (1 ng/mL=16.36 IU/mL for IL-2).

### Flow Cytometry

Cells were first stained with LIVE/DEAD™ Fixable Aqua (Thermo Fisher Scientific, Inc.), followed by staining with antibodies against various surface markers. Samples were then acquired by the CytoFLEX LX Flow cytometer. All data were analyzed with FlowJo V10 software.

### Results and Analysis

As an alternative to flasks and bags of the young TIL process, G-Rex® (gas permeable rapid expansion) is a production platform created specifically to produce immune cells, which is easy to scale up, scale out and compatible with cGMP conditions that are required for clinical trials. Initial TIL growth (pre-REP) has been compared using metastatic melanoma tumors cultured in G-Rex®10 devices vs in 24-well plates². G-Rex® devices were used in our studies as the pre-REP TIL culture vessels.

We first used 4 matched sets of tumor samples (T5101001, T5101002, T5101004, and T5101005) for pre-REP using G-Rex®10M. Final pre-REP cell yields ranged from (15.60±25.09)×10⁶ cells to (27.54±22.60)×10⁶ cells for 4 donors.

Phenotyping of pre-REP TILs showed that a larger portion of CD4⁺ cells and CD8⁺ cells in TILs had a central memory phenotype, T_{CM} (central memory T cells), rather than effector phenotype, T_{EM} (effector memory T cells).

Glucose and lactate levels, as well as IL-2 consumption in pre-REP cultures were measured to optimize the feeding. We next scaled up the process to use the G-Rex®100M device (bottom surface area of 100 cm², 1000 mL volume capacity). Tumor fragments were used to start pre-REP culture for 3 donors (T5101009, T5101010, and T5101013). The pre-REP runs of T5101009 and T5101010 were performed before the determination of culturing time and were stopped on Day 17 or Day 20, respectively, without any media addition or exchange. For T5101013, a cell count was performed, and fresh media was added on or around day 8, day 9, day 10 or day 1. Glucose levels began to decline after day 6 for T5101010 and T5101013, where there was greater cell expansion compared to T5101009. Greater lactate production by T5101010 was detected by day 3 and continued to increase throughout the culture. For T5101013, addition of fresh media did appear to prevent the lactate levels from further increase. Such media addition will be particularly important for donors with rapidly growing TILs to keep lactate at low levels tolerated by T cells.

We further evaluated IL-2 consumption in pre-REP cultures. The IL-2 protein concentrations were measured with an ELISA kit, and the concentration (pg/mL) was converted to IU/mL (IL-2: 1 ng/mL=16.36 IU/mL). During the first 6 days of the 2 pre-REP runs analyzed, we did not change or add culture medium since the total number of T cells was small at the early stage. However, we still observed an approximately 50% decrease of IL-2 levels although the yields of both T5101001 and T5101002 pre-REP TILs on day 6 were less than 2×10⁶.

We seeded TILs that were derived from a single tumor fragment in 24-well plates into the G-Rex®10M devices, and further cultured them for additional 8 days without medium change/addition in the first 6 days. We found consistent, ˜50% decrease in IL-2 levels after 6 days in culture medium samples from both T5101001 and T5101002, although their expansion differed markedly (53.8-fold and 20.3-fold for T5101001-5×10⁶ and T5101001-10×10⁶, respectively; 3.18-fold and 3.57-fold for T5101002-5×10⁶ and T5101002-10×10⁶, respectively). Therefore, T cell growth did not seem to be a determining factor for IL-2 concentration decline in pre-REP TIL cultures.

Furthermore, we investigated the IL-2 consumption in G-Rex®100M devices during the pre-REP stage (The same sets of samples as used for glucose and lactate measurement). In this analysis, Donor T5101009 and Donor T5101010 had markedly different pre-REP TIL yields (2.2×10⁶ vs 93×10⁶), yet the pre-REP runs displayed comparable IL-2 decline curves. It is worth noting that no medium change/addition was conducted during the whole pre-REP stage for these two donors. It resulted that there remained ˜20% of the original IL-2 concentration on day 14, no matter how robustly T cells expanded.

### Pre-REP Procedure

Day 0—Tumor fragmentation and primary TIL culture. Briefly, tumor is dissected into fragments ˜2-3 mm in each dimension, with necrotic, hemorrhagic, and fatty deposits removed. Culture 1, 2, 3, 4 or 5 fragments per a G-Rex®10M culture device in a 10 mL, 20 mL, 25 mL, 30 mL, 50 mL or 100 mL of initial culture volume. For a total number of fragments up to 100, one to two G-Rex®100M culture device may be used with 100 mL, 200 mL, 250 mL, 500 mL, or 1000 mL initial culture volume. Total pre-REP culture media for the whole process may be prepared on Day 0 using 500 or 1000 mL of pre-REP basal media supplemented with 300 IU/mL, 1000 IU/mL, 3000 IU/mL, or 6000 IU/mL IL-2. In one embodiment, the IL-2 concentration is 300 IU/mL. In another embodiment, the IL-2 concentration is 1000 IU/mL. In yet another embodiment, the IL-2 concentration is 3000 IU/mL. In still another embodiment, the IL-2 concentration is 6000 IU/mL.

Day 8, 9, 10 or 11—Media addition or exchange. Pre-REP CM (culture medium) made on Day 0 is warmed up at RT or 37° C. before being used. If using G-Rex®10M or G-Rex®100M devices, a total volume of 1-fold, 2-fold or 3-fold of the volume of the initial pre-REP CM is added or exchanged to each culture device. Culture devices are returned to incubator for continuation of TIL culture.

Day 13, 14, 15, 16 or 17—Pre-REP TIL harvest. 100 or 200 mL of harvesting solution is made using Plasma-Lyte A (Multiple Electrolytes for Injection) and human serum albumin (HSA) to achieve a final concentration of 0.5%, 0.7%, 1%, 1.5%, 2% or 5% HSA solution. TILs are harvested manually. Cell suspension is collected and is filtered through a cell strainer. The G-Rex® device is washed twice to make sure all TILs are collected.

Cells are centrifuged 200, 300 or 400 g for 15 or 30 min at 4° C. Supernatant is discarded and cell pellets are resuspended and combined using harvesting solution containing HSA in Plasma-Lyte A. Cell count is conducted using NC200 automated cell counter and cells are cryopreserved.

The below culture conditions are used.

### Culture Condition (A-i):

Day 0—The initial culture volume is 10 mL for G-Rex®10M (or 100 mL for G-Rex®100M). In other words, the initial culture volume for the first expansion is 10% of the volume capacity of the cell culture container/system.

Day 8—A cell culture medium having a volume of I-fold of the volume of the initial culture volume is added or exchanged.

Day 13—Pre-REP TIL harvest.

### Culture Condition (A-ii):

Day 0—The initial culture volume is 20 mL for G-Rex®10M (or 200 mL for G-Rex®100M). In other words, the initial culture volume for the first expansion is 20% of the volume capacity of the cell culture container/system.

Day 8—Cell culture medium having a volume of 1-fold of the volume of the initial culture volume is added or exchanged.

Day 13—Pre-REP TIL harvest.

### Culture Condition (A-iii):

Day 0—The initial culture volume is 25 mL for G-Rex®10M (or 250 mL for G-Rex®100M). In other words, the initial culture volume for the first expansion is 25% of the volume capacity of the cell culture container/system.

Day 8—Cell culture medium having a volume of 1-fold of the volume of the initial culture volume is added or exchanged.

Day 13—Pre-REP TIL harvest.

### Culture Condition (A-iv):

Day 0—The initial culture volume is 30 mL for G-Rex®10M (or 300 mL for G-Rex®100M). In other words, the initial culture volume for the first expansion is 30% of the volume capacity of the cell culture container/system.

Day 8—Cell culture medium having a volume of 1-fold of the volume of the initial culture volume is added or exchanged.

Day 13—Pre-REP TIL harvest.

### Culture Condition (A-v):

Day 0—The initial culture volume is 50 mL for G-Rex®10M (or 500 mL for G-Rex®100M). In other words, the initial culture volume for the first expansion is 50% of the volume capacity of the cell culture container/system.

Day 8—Cell culture medium having a volume of 1-fold of the volume of the initial culture volume is added or exchanged.

Day 13—Pre-REP TIL harvest.

### Culture Condition (A-vi):

Day 0—The initial culture volume is 100 mL for G-Rex®10M (or 1000 mL for G-Rex®100M). In other words, the initial culture volume for the first expansion is 100% of the volume capacity of the cell culture container/system.

Day 8—Cell culture medium having a volume of 1-fold of the volume of the initial culture volume is exchanged.

Day 13—Pre-REP TIL harvest.

### Culture Condition (A-vii):

Day 0—The initial culture volume is 10 mL for G-Rex®10M (or 100 mL for G-Rex®100M). In other words, the initial culture volume for the first expansion is 10% of the volume capacity of the cell culture container/system.

Day 8—A cell culture medium having a volume of 2-fold of the volume of the initial culture volume is added or exchanged.

Day 13—Pre-REP TIL harvest.

### Culture Condition (A-viii):

Day 0—The initial culture volume is 20 mL for G-Rex®10M (or 200 mL for G-Rex®100M). In other words, the initial culture volume for the first expansion is 20% of the volume capacity of the cell culture container/system.

Day 8—Cell culture medium having a volume of 2-fold of the volume of the initial culture volume is added or exchanged.

Day 13—Pre-REP TIL harvest.

### Culture Condition (A-ix):

Day 0—The initial culture volume is 25 mL for G-Rex®10M (or 250 mL for G-Rex®100M). In other words, the initial culture volume for the first expansion is 25% of the volume capacity of the cell culture container/system.

Day 8—Cell culture medium having a volume of 2-fold of the volume of the initial culture volume is added or exchanged.

Day 13—Pre-REP TIL harvest.

### Culture Condition (A-x):

Day 0—The initial culture volume is 30 mL for G-Rex®10M (or 300 mL for G-Rex®100M). In other words, the initial culture volume for the first expansion is 30% of the volume capacity of the cell culture container/system.

Day 8—Cell culture medium having a volume of 2-fold of the volume of the initial culture volume is added or exchanged.

Day 13—Pre-REP TIL harvest.

### Culture Condition (A-xi):

Day 0—The initial culture volume is 50 mL for G-Rex®10M (or 500 mL for G-Rex®100M). In other words, the initial culture volume for the first expansion is 50% of the volume capacity of the cell culture container/system.

Day 8—Cell culture medium having a volume of 2-fold of the volume of the initial culture volume is exchanged.

Day 13—Pre-REP TIL harvest.

### Culture Condition (A-xii):

Day 0—The initial culture volume is 10 mL for G-Rex®10M (or 100 mL for G-Rex®100M). In other words, the initial culture volume for the first expansion is 10% of the volume capacity of the cell culture container/system.

Day 8—A cell culture medium having a volume of 3-fold of the volume of the initial culture volume is added or exchanged.

Day 13—Pre-REP TIL harvest.

### Culture Condition (A-xiii):

Day 0—The initial culture volume is 20 mL for G-Rex®10M (or 200 mL for G-Rex® 100M). In other words, the initial culture volume for the first expansion is 20% of the volume capacity of the cell culture container/system.

Day 8—Cell culture medium having a volume of 3-fold of the volume of the initial culture volume is added or exchanged.

Day 13—Pre-REP TIL harvest.

### Culture Condition (A-xiv):

Day 0—The initial culture volume is 25 mL for G-Rex®10M (or 250 mL for G-Rex®100M). In other words, the initial culture volume for the first expansion is 25% of the volume capacity of the cell culture container/system.

Day 8—Cell culture medium having a volume of 3-fold of the volume of the initial culture volume is added or exchanged.

Day 13—Pre-REP TIL harvest.

### Culture Condition (A-xv):

Day 0—The initial culture volume is 30 mL for G-Rex®10M (or 300 mL for G-Rex® 100M). In other words, the initial culture volume for the first expansion is 30% of the volume capacity of the cell culture container/system.

Day 8—Cell culture medium having a volume of 3-fold of the volume of the initial culture volume is exchanged.

Day 13—Pre-REP TIL harvest.

### Culture Condition (B-i):

Day 0—The initial culture volume is 10 mL for G-Rex®10M (or 100 mL for G-Rex® 100M). In other words, the initial culture volume for the first expansion is 10% of the volume capacity of the cell culture container/system.

Day 9—A cell culture medium having a volume of 1-fold of the volume of the initial culture volume is added or exchanged.

Day 13—Pre-REP TIL harvest.

### Culture Condition (B-ii):

Day 0—The initial culture volume is 20 mL for G-Rex®10M (or 200 mL for G-Rex®100M). In other words, the initial culture volume for the first expansion is 20% of the volume capacity of the cell culture container/system.

Day 9—Cell culture medium having a volume of 1-fold of the volume of the initial culture volume is added or exchanged.

Day 13—Pre-REP TIL harvest.

### Culture Condition (B-iii):

Day 0—The initial culture volume is 25 mL for G-Rex®10M (or 250 mL for G-Rex®100M). In other words, the initial culture volume for the first expansion is 25% of the volume capacity of the cell culture container/system.

Day 9—Cell culture medium having a volume of 1-fold of the volume of the initial culture volume is added or exchanged.

Day 13—Pre-REP TIL harvest.

### Culture Condition (B-iv):

Day 0—The initial culture volume is 30 mL for G-Rex®10M (or 300 mL for G-Rex®100M). In other words, the initial culture volume for the first expansion is 30% of the volume capacity of the cell culture container/system.

Day 9—Cell culture medium having a volume of 1-fold of the volume of the initial culture volume is added or exchanged.

Day 13—Pre-REP TIL harvest.

### Culture Condition (B-v):

Day 0—The initial culture volume is 50 mL for G-Rex®10M (or 500 mL for G-Rex®100M). In other words, the initial culture volume for the first expansion is 50% of the volume capacity of the cell culture container/system.

Day 9—Cell culture medium having a volume of 1-fold of the volume of the initial culture volume is added or exchanged.

Day 13—Pre-REP TIL harvest.

### Culture Condition (B-vi):

Day 0—The initial culture volume is 100 mL for G-Rex®10M (or 1000 mL for G-Rex®100M). In other words, the initial culture volume for the first expansion is 100% of the volume capacity of the cell culture container/system.

Day 9—Cell culture medium having a volume of 1-fold of the volume of the initial culture volume is exchanged.

Day 13—Pre-REP TIL harvest.

### Culture Condition (B-vii):

Day 0—The initial culture volume is 10 mL for G-Rex®10M (or 100 mL for G-Rex®100M). In other words, the initial culture volume for the first expansion is 10% of the volume capacity of the cell culture container/system.

Day 9—A cell culture medium having a volume of 2-fold of the volume of the initial culture volume is added or exchanged.

Day 13—Pre-REP TIL harvest.

### Culture Condition (B-viii):

Day 0—The initial culture volume is 20 mL for G-Rex®10M (or 200 mL for G-Rex®100M). In other words, the initial culture volume for the first expansion is 20% of the volume capacity of the cell culture container/system.

Day 9—Cell culture medium having a volume of 2-fold of the volume of the initial culture volume is added or exchanged.

Day 13—Pre-REP TIL harvest.

### Culture Condition (B-ix):

Day 0—The initial culture volume is 25 mL for G-Rex®10M (or 250 mL for G-Rex®100M). In other words, the initial culture volume for the first expansion is 25% of the volume capacity of the cell culture container/system.

Day 9—Cell culture medium having a volume of 2-fold of the volume of the initial culture volume is added or exchanged.

Day 13—Pre-REP TIL harvest.

### Culture Condition (B-x):

Day 0—The initial culture volume is 30 mL for G-Rex®10M (or 300 mL for G-Rex®100M). In other words, the initial culture volume for the first expansion is 30% of the volume capacity of the cell culture container/system.

Day 9—Cell culture medium having a volume of 2-fold of the volume of the initial culture volume is added or exchanged.

Day 13—Pre-REP TIL harvest.

### Culture Condition (B-xi):

Day 0—The initial culture volume is 50 mL for G-Rex®10M (or 500 mL for G-Rex®100M). In other words, the initial culture volume for the first expansion is 50% of the volume capacity of the cell culture container/system.

Day 9—Cell culture medium having a volume of 2-fold of the volume of the initial culture volume is exchanged.

Day 13—Pre-REP TIL harvest.

### Culture Condition (B-xii):

Day 0—The initial culture volume is 10 mL for G-Rex®10M (or 100 mL for G-Rex®100M). In other words, the initial culture volume for the first expansion is 10% of the volume capacity of the cell culture container/system.

Day 9—A cell culture medium having a volume of 3-fold of the volume of the initial culture volume is added or exchanged.

Day 13—Pre-REP TIL harvest.

### Culture Condition (B-xiii):

Day 0—The initial culture volume is 20 mL for G-Rex®10M (or 200 mL for G-Rex®100M). In other words, the initial culture volume for the first expansion is 20% of the volume capacity of the cell culture container/system.

Day 9—Cell culture medium having a volume of 3-fold of the volume of the initial culture volume is added or exchanged.

Day 13—Pre-REP TIL harvest.

### Culture Condition (B-xiv):

Day 0—The initial culture volume is 25 mL for G-Rex®10M (or 250 mL for G-Rex®100M). In other words, the initial culture volume for the first expansion is 25% of the volume capacity of the cell culture container/system.

Day 9—Cell culture medium having a volume of 3-fold of the volume of the initial culture volume is added or exchanged.

Day 13—Pre-REP TIL harvest.

### Culture Condition (B-xv):

Day 0—The initial culture volume is 30 mL for G-Rex®10M (or 300 mL for G-Rex®100M). In other words, the initial culture volume for the first expansion is 30% of the volume capacity of the cell culture container/system.

Day 9—Cell culture medium having a volume of 3-fold of the volume of the initial culture volume is exchanged.

Day 13—Pre-REP TIL harvest.

### Culture Condition (C-i):

Day 0—The initial culture volume is 10 mL for G-Rex®10M (or 100 mL for G-Rex®100M). In other words, the initial culture volume for the first expansion is 10% of the volume capacity of the cell culture container/system.

Day 10—A cell culture medium having a volume of 1-fold of the volume of the initial culture volume is added or exchanged.

Day 13—Pre-REP TIL harvest.

### Culture Condition (C-ii):

Day 0—The initial culture volume is 20 mL for G-Rex®10M (or 200 mL for G-Rex®100M). In other words, the initial culture volume for the first expansion is 20% of the volume capacity of the cell culture container/system.

Day 10—Cell culture medium having a volume of 1-fold of the volume of the initial culture volume is added or exchanged.

Day 13—Pre-REP TIL harvest.

### Culture Condition (C-iii):

Day 0—The initial culture volume is 25 mL for G-Rex®10M (or 250 mL for G-Rex®100M). In other words, the initial culture volume for the first expansion is 25% of the volume capacity of the cell culture container/system.

Day 10—Cell culture medium having a volume of 1-fold of the volume of the initial culture volume is added or exchanged.

Day 13—Pre-REP TIL harvest.

### Culture Condition (C-iv):

Day 0—The initial culture volume is 30 mL for G-Rex®10M (or 300 mL for G-Rex®100M). In other words, the initial culture volume for the first expansion is 30% of the volume capacity of the cell culture container/system.

Day 10—Cell culture medium having a volume of 1-fold of the volume of the initial culture volume is added or exchanged.

Day 13—Pre-REP TIL harvest.

### Culture Condition (C-v):

Day 0—The initial culture volume is 50 mL for G-Rex®10M (or 500 mL for G-Rex®100M). In other words, the initial culture volume for the first expansion is 50% of the volume capacity of the cell culture container/system.

Day 10—Cell culture medium having a volume of 1-fold of the volume of the initial culture volume is added or exchanged.

Day 13—Pre-REP TIL harvest.

### Culture Condition (C-vi):

Day 0—The initial culture volume is 100 mL for G-Rex®10M (or 1000 mL for G-Rex®100M). In other words, the initial culture volume for the first expansion is 100% of the volume capacity of the cell culture container/system.

Day 10—Cell culture medium having a volume of 1-fold of the volume of the initial culture volume is exchanged.

Day 13—Pre-REP TIL harvest.

### Culture Condition (C-vii):

Day 0—The initial culture volume is 10 mL for G-Rex®10M (or 100 mL for G-Rex®100M). In other words, the initial culture volume for the first expansion is 10% of the volume capacity of the cell culture container/system.

Day 10—A cell culture medium having a volume of 2-fold of the volume of the initial culture volume is added or exchanged.

Day 13—Pre-REP TIL harvest.

### Culture Condition (C-viii):

Day 0—The initial culture volume is 20 mL for G-Rex®10M (or 200 mL for G-Rex®100M). In other words, the initial culture volume for the first expansion is 20% of the volume capacity of the cell culture container/system.

Day 10—Cell culture medium having a volume of 2-fold of the volume of the initial culture volume is added or exchanged.

Day 13—Pre-REP TIL harvest.

### Culture Condition (C-ix):

Day 0—The initial culture volume is 25 mL for G-Rex®10M (or 250 mL for G-Rex®100M). In other words, the initial culture volume for the first expansion is 25% of the volume capacity of the cell culture container/system.

Day 10—Cell culture medium having a volume of 2-fold of the volume of the initial culture volume is added or exchanged.

Day 13—Pre-REP TIL harvest.

### Culture Condition (C-x):

Day 0—The initial culture volume is 30 mL for G-Rex®10M (or 300 mL for G-Rex® 100M). In other words, the initial culture volume for the first expansion is 30% of the volume capacity of the cell culture container/system.

Day 10—Cell culture medium having a volume of 2-fold of the volume of the initial culture volume is added or exchanged.

Day 13—Pre-REP TIL harvest.

### Culture Condition (C-xi):

Day 0—The initial culture volume is 50 mL for G-Rex®10M (or 500 mL for G-Rex®100M). In other words, the initial culture volume for the first expansion is 50% of the volume capacity of the cell culture container/system.

Day 10—Cell culture medium having a volume of 2-fold of the volume of the initial culture volume is exchanged.

Day 13—Pre-REP TIL harvest.

### Culture Condition (C-xii):

Day 0—The initial culture volume is 10 mL for G-Rex®10M (or 100 mL for G-Rex®100M). In other words, the initial culture volume for the first expansion is 10% of the volume capacity of the cell culture container/system.

Day 10—A cell culture medium having a volume of 3-fold of the volume of the initial culture volume is added or exchanged.

Day 13—Pre-REP TIL harvest.

### Culture Condition (C-xiii):

Day 0—The initial culture volume is 20 mL for G-Rex®10M (or 200 mL for G-Rex®100M). In other words, the initial culture volume for the first expansion is 20% of the volume capacity of the cell culture container/system.

Day 10—Cell culture medium having a volume of 3-fold of the volume of the initial culture volume is added or exchanged.

Day 13—Pre-REP TIL harvest.

### Culture Condition (C-xiv):

Day 0—The initial culture volume is 25 mL for G-Rex®10M (or 250 mL for G-Rex®100M). In other words, the initial culture volume for the first expansion is 25% of the volume capacity of the cell culture container/system.

Day 10—Cell culture medium having a volume of 3-fold of the volume of the initial culture volume is added or exchanged.

Day 13—Pre-REP TIL harvest.

### Culture Condition (C-xv):

Day 0—The initial culture volume is 30 mL for G-Rex®10M (or 300 mL for G-Rex®100M). In other words, the initial culture volume for the first expansion is 30% of the volume capacity of the cell culture container/system.

Day 10—Cell culture medium having a volume of 3-fold of the volume of the initial culture volume is exchanged.

Day 13—Pre-REP TIL harvest.

### Culture Condition (D-i):

Day 0—The initial culture volume is 10 mL for G-Rex®10M (or 100 mL for G-Rex®100M). In other words, the initial culture volume for the first expansion is 10% of the volume capacity of the cell culture container/system.

Day 11—A cell culture medium having a volume of 1-fold of the volume of the initial culture volume is added or exchanged.

Day 13—Pre-REP TIL harvest.

### Culture Condition (D-ii):

Day 0—The initial culture volume is 20 mL for G-Rex®10M (or 200 mL for G-Rex®100M). In other words, the initial culture volume for the first expansion is 20% of the volume capacity of the cell culture container/system.

Day 11—Cell culture medium having a volume of I-fold of the volume of the initial culture volume is added or exchanged.

Day 13—Pre-REP TIL harvest.

### Culture Condition (D-iii):

Day 0—The initial culture volume is 25 mL for G-Rex®10M (or 250 mL for G-Rex®100M). In other words, the initial culture volume for the first expansion is 25% of the volume capacity of the cell culture container/system.

Day 11—Cell culture medium having a volume of I-fold of the volume of the initial culture volume is added or exchanged.

Day 13—Pre-REP TIL harvest.

### Culture Condition (D-iv):

Day 0—The initial culture volume is 30 mL for G-Rex®10M (or 300 mL for G-Rex®100M). In other words, the initial culture volume for the first expansion is 30% of the volume capacity of the cell culture container/system.

Day 11—Cell culture medium having a volume of 1-fold of the volume of the initial culture volume is added or exchanged.

Day 13—Pre-REP TIL harvest.

### Culture Condition (D-v):

Day 0—The initial culture volume is 50 mL for G-Rex®10M (or 500 mL for G-Rex®100M). In other words, the initial culture volume for the first expansion is 50% of the volume capacity of the cell culture container/system.

Day 11—Cell culture medium having a volume of 1-fold of the volume of the initial culture volume is added or exchanged.

Day 13—Pre-REP TIL harvest.

### Culture Condition (D-vi):

Day 0—The initial culture volume is 100 mL for G-Rex®10M (or 1000 mL for G-Rex®100M). In other words, the initial culture volume for the first expansion is 100% of the volume capacity of the cell culture container/system.

Day 11—Cell culture medium having a volume of 1-fold of the volume of the initial culture volume is exchanged.

Day 13—Pre-REP TIL harvest.

### Culture Condition (D-vii):

Day 0—The initial culture volume is 10 mL for G-Rex®10M (or 100 mL for G-Rex®100M). In other words, the initial culture volume for the first expansion is 10% of the volume capacity of the cell culture container/system.

Day 11—A cell culture medium having a volume of 2-fold of the volume of the initial culture volume is added or exchanged.

Day 13—Pre-REP TIL harvest.

### Culture Condition (D-viii):

Day 0—The initial culture volume is 20 mL for G-Rex®10M (or 200 mL for G-Rex®100M). In other words, the initial culture volume for the first expansion is 20% of the volume capacity of the cell culture container/system.

Day 11—Cell culture medium having a volume of 2-fold of the volume of the initial culture volume is added or exchanged.

Day 13—Pre-REP TIL harvest.

### Culture Condition (D-ix):

Day 0—The initial culture volume is 25 mL for G-Rex®10M (or 250 mL for G-Rex®100M). In other words, the initial culture volume for the first expansion is 25% of the volume capacity of the cell culture container/system.

Day 11—Cell culture medium having a volume of 2-fold of the volume of the initial culture volume is added or exchanged.

Day 13—Pre-REP TIL harvest.

### Culture Condition (D-x):

Day 0—The initial culture volume is 30 mL for G-Rex®10M (or 300 mL for G-Rex®100M). In other words, the initial culture volume for the first expansion is 30% of the volume capacity of the cell culture container/system.

Day 11—Cell culture medium having a volume of 2-fold of the volume of the initial culture volume is added or exchanged.

Day 13—Pre-REP TIL harvest.

### Culture Condition (D-xi):

Day 0—The initial culture volume is 50 mL for G-Rex®10M (or 500 mL for G-Rex®100M). In other words, the initial culture volume for the first expansion is 50% of the volume capacity of the cell culture container/system.

Day 11—Cell culture medium having a volume of 2-fold of the volume of the initial culture volume is exchanged.

Day 13—Pre-REP TIL harvest.

### Culture Condition (D-xii):

Day 0—The initial culture volume is 10 mL for G-Rex®10M (or 100 mL for G-Rex® 100M). In other words, the initial culture volume for the first expansion is 10% of the volume capacity of the cell culture container/system.

Day 11—A cell culture medium having a volume of 3-fold of the volume of the initial culture volume is added or exchanged.

Day 13—Pre-REP TIL harvest.

### Culture Condition (D-xiii):

Day 0—The initial culture volume is 20 mL for G-Rex®10M (or 200 mL for G-Rex®100M). In other words, the initial culture volume for the first expansion is 20% of the volume capacity of the cell culture container/system.

Day 11—Cell culture medium having a volume of 3-fold of the volume of the initial culture volume is added or exchanged.

Day 13—Pre-REP TIL harvest.

### Culture Condition (D-xiv):

Day 0—The initial culture volume is 25 mL for G-Rex®10M (or 250 mL for G-Rex®100M). In other words, the initial culture volume for the first expansion is 25% of the volume capacity of the cell culture container/system.

Day 11—Cell culture medium having a volume of 3-fold of the volume of the initial culture volume is added or exchanged.

Day 13—Pre-REP TIL harvest.

### Culture Condition (D-xv):

Day 0—The initial culture volume is 30 mL for G-Rex®10M (or 300 mL for G-Rex®100M). In other words, the initial culture volume for the first expansion is 30% of the volume capacity of the cell culture container/system.

Day 11—Cell culture medium having a volume of 3-fold of the volume of the initial culture volume is exchanged.

Day 13—Pre-REP TIL harvest.

### Culture Condition (E-i):

Day 0—The initial culture volume is 10 mL for G-Rex®10M (or 100 mL for G-Rex®100M). In other words, the initial culture volume for the first expansion is 10% of the volume capacity of the cell culture container/system.

Day 8—A cell culture medium having a volume of 1-fold of the volume of the initial culture volume is added or exchanged.

Day 14—Pre-REP TIL harvest.

### Culture Condition (E-ii):

Day 0—The initial culture volume is 20 mL for G-Rex®10M (or 200 mL for G-Rex®100M). In other words, the initial culture volume for the first expansion is 20% of the volume capacity of the cell culture container/system.

Day 8—Cell culture medium having a volume of 1-fold of the volume of the initial culture volume is added or exchanged.

Day 14—Pre-REP TIL harvest.

### Culture Condition (E-iii):

Day 0—The initial culture volume is 25 mL for G-Rex®10M (or 250 mL for G-Rex®100M). In other words, the initial culture volume for the first expansion is 25% of the volume capacity of the cell culture container/system.

Day 8—Cell culture medium having a volume of 1-fold of the volume of the initial culture volume is added or exchanged.

Day 14—Pre-REP TIL harvest.

### Culture Condition (E-iv):

Day 0—The initial culture volume is 30 mL for G-Rex®10M (or 300 mL for G-Rex®100M). In other words, the initial culture volume for the first expansion is 30% of the volume capacity of the cell culture container/system.

Day 8—Cell culture medium having a volume of 1-fold of the volume of the initial culture volume is added or exchanged.

Day 14—Pre-REP TIL harvest.

### Culture Condition (E-v):

Day 0—The initial culture volume is 50 mL for G-Rex®10M (or 500 mL for G-Rex®100M). In other words, the initial culture volume for the first expansion is 50% of the volume capacity of the cell culture container/system.

Day 8—Cell culture medium having a volume of 1-fold of the volume of the initial culture volume is added or exchanged.

Day 14—Pre-REP TIL harvest.

### Culture Condition (E-vi):

Day 0—The initial culture volume is 100 mL for G-Rex®10M (or 1000 mL for G-Rex®100M). In other words, the initial culture volume for the first expansion is 100% of the volume capacity of the cell culture container/system.

Day 8—Cell culture medium having a volume of 1-fold of the volume of the initial culture volume is exchanged.

Day 14—Pre-REP TIL harvest.

### Culture Condition (E-vii):

Day 0—The initial culture volume is 10 mL for G-Rex®10M (or 100 mL for G-Rex®100M). In other words, the initial culture volume for the first expansion is 10% of the volume capacity of the cell culture container/system.

Day 8—A cell culture medium having a volume of 2-fold of the volume of the initial culture volume is added or exchanged.

Day 14—Pre-REP TIL harvest.

### Culture Condition (E-viii):

Day 0—The initial culture volume is 20 mL for G-Rex®10M (or 200 mL for G-Rex®100M). In other words, the initial culture volume for the first expansion is 20% of the volume capacity of the cell culture container/system.

Day 8—Cell culture medium having a volume of 2-fold of the volume of the initial culture volume is added or exchanged.

Day 14—Pre-REP TIL harvest.

### Culture Condition (E-ix):

Day 0—The initial culture volume is 25 mL for G-Rex®10M (or 250 mL for G-Rex®100M). In other words, the initial culture volume for the first expansion is 25% of the volume capacity of the cell culture container/system.

Day 8—Cell culture medium having a volume of 2-fold of the volume of the initial culture volume is added or exchanged.

Day 14—Pre-REP TIL harvest.

### Culture Condition (E-x):

Day 0—The initial culture volume is 30 mL for G-Rex®10M (or 300 mL for G-Rex®100M). In other words, the initial culture volume for the first expansion is 30% of the volume capacity of the cell culture container/system.

Day 8—Cell culture medium having a volume of 2-fold of the volume of the initial culture volume is added or exchanged.

Day 14—Pre-REP TIL harvest.

### Culture Condition (E-xi):

Day 0—The initial culture volume is 50 mL for G-Rex®10M (or 500 mL for G-Rex®100M). In other words, the initial culture volume for the first expansion is 50% of the volume capacity of the cell culture container/system.

Day 8—Cell culture medium having a volume of 2-fold of the volume of the initial culture volume is exchanged.

Day 14—Pre-REP TIL harvest.

### Culture Condition (E-xii):

Day 0—The initial culture volume is 10 mL for G-Rex®10M (or 100 mL for G-Rex®100M). In other words, the initial culture volume for the first expansion is 10% of the volume capacity of the cell culture container/system.

Day 8—A cell culture medium having a volume of 3-fold of the volume of the initial culture volume is added or exchanged.

Day 14—Pre-REP TIL harvest.

### Culture Condition (E-xiii):

Day 0—The initial culture volume is 20 mL for G-Rex®10M (or 200 mL for G-Rex®100M). In other words, the initial culture volume for the first expansion is 20% of the volume capacity of the cell culture container/system.

Day 8—Cell culture medium having a volume of 3-fold of the volume of the initial culture volume is added or exchanged.

Day 14—Pre-REP TIL harvest.

### Culture Condition (E-xiv):

Day 0—The initial culture volume is 25 mL for G-Rex®10M (or 250 mL for G-Rex®100M). In other words, the initial culture volume for the first expansion is 25% of the volume capacity of the cell culture container/system.

Day 8—Cell culture medium having a volume of 3-fold of the volume of the initial culture volume is added or exchanged.

Day 14—Pre-REP TIL harvest.

### Culture Condition (E-xv):

Day 0—The initial culture volume is 30 mL for G-Rex®10M (or 300 mL for G-Rex®100M). In other words, the initial culture volume for the first expansion is 30% of the volume capacity of the cell culture container/system.

Day 8—Cell culture medium having a volume of 3-fold of the volume of the initial culture volume is exchanged.

Day 14—Pre-REP TIL harvest.

### Culture Condition (F-i):

Day 0—The initial culture volume is 10 mL for G-Rex®10M (or 100 mL for G-Rex®100M). In other words, the initial culture volume for the first expansion is 10% of the volume capacity of the cell culture container/system.

Day 9—A cell culture medium having a volume of 1-fold of the volume of the initial culture volume is added or exchanged.

Day 14—Pre-REP TIL harvest.

### Culture Condition (F-ii):

Day 0—The initial culture volume is 20 mL for G-Rex®10M (or 200 mL for G-Rex®100M). In other words, the initial culture volume for the first expansion is 20% of the volume capacity of the cell culture container/system.

Day 9—Cell culture medium having a volume of 1-fold of the volume of the initial culture volume is added or exchanged.

Day 14—Pre-REP TIL harvest.

### Culture Condition (F-iii):

Day 0—The initial culture volume is 25 mL for G-Rex®10M (or 250 mL for G-Rex®100M). In other words, the initial culture volume for the first expansion is 25% of the volume capacity of the cell culture container/system.

Day 9—Cell culture medium having a volume of 1-fold of the volume of the initial culture volume is added or exchanged.

Day 14—Pre-REP TIL harvest.

### Culture Condition (F-iv):

Day 0—The initial culture volume is 30 mL for G-Rex®10M (or 300 mL for G-Rex®100M). In other words, the initial culture volume for the first expansion is 30% of the volume capacity of the cell culture container/system.

Day 9—Cell culture medium having a volume of 1-fold of the volume of the initial culture volume is added or exchanged.

Day 14—Pre-REP TIL harvest.

### Culture Condition (F-v):

Day 0—The initial culture volume is 50 mL for G-Rex®10M (or 500 mL for G-Rex®100M). In other words, the initial culture volume for the first expansion is 50% of the volume capacity of the cell culture container/system.

Day 9—Cell culture medium having a volume of 1-fold of the volume of the initial culture volume is added or exchanged.

Day 14—Pre-REP TIL harvest.

### Culture Condition (F-vi):

Day 0—The initial culture volume is 100 mL for G-Rex®10M (or 1000 mL for G-Rex®100M). In other words, the initial culture volume for the first expansion is 100% of the volume capacity of the cell culture container/system.

Day 9—Cell culture medium having a volume of 1-fold of the volume of the initial culture volume is exchanged.

Day 14—Pre-REP TIL harvest.

### Culture Condition (F-vii):

Day 0—The initial culture volume is 10 mL for G-Rex®10M (or 100 mL for G-Rex®100M). In other words, the initial culture volume for the first expansion is 10% of the volume capacity of the cell culture container/system.

Day 9—A cell culture medium having a volume of 2-fold of the volume of the initial culture volume is added or exchanged.

Day 14—Pre-REP TIL harvest.

### Culture Condition (F-viii):

Day 0—The initial culture volume is 20 mL for G-Rex®10M (or 200 mL for G-Rex®100M). In other words, the initial culture volume for the first expansion is 20% of the volume capacity of the cell culture container/system.

Day 9—Cell culture medium having a volume of 2-fold of the volume of the initial culture volume is added or exchanged.

Day 14—Pre-REP TIL harvest.

### Culture Condition (F-ix):

Day 0—The initial culture volume is 25 mL for G-Rex®10M (or 250 mL for G-Rex®100M). In other words, the initial culture volume for the first expansion is 25% of the volume capacity of the cell culture container/system.

Day 9—Cell culture medium having a volume of 2-fold of the volume of the initial culture volume is added or exchanged.

Day 14—Pre-REP TIL harvest.

### Culture Condition (F-x):

Day 0—The initial culture volume is 30 mL for G-Rex®10M (or 300 mL for G-Rex®100M). In other words, the initial culture volume for the first expansion is 30% of the volume capacity of the cell culture container/system.

Day 9—Cell culture medium having a volume of 2-fold of the volume of the initial culture volume is added or exchanged.

Day 14—Pre-REP TIL harvest.

### Culture Condition (F-xi):

Day 0—The initial culture volume is 50 mL for G-Rex®10M (or 500 mL for G-Rex®100M). In other words, the initial culture volume for the first expansion is 50% of the volume capacity of the cell culture container/system.

Day 9—Cell culture medium having a volume of 2-fold of the volume of the initial culture volume is exchanged.

Day 14—Pre-REP TIL harvest.

### Culture Condition (F-xii):

Day 0—The initial culture volume is 10 mL for G-Rex®10M (or 100 mL for G-Rex®100M). In other words, the initial culture volume for the first expansion is 10% of the volume capacity of the cell culture container/system.

Day 9—A cell culture medium having a volume of 3-fold of the volume of the initial culture volume is added or exchanged.

Day 14—Pre-REP TIL harvest.

### Culture Condition (F-xiii):

Day 0—The initial culture volume is 20 mL for G-Rex®10M (or 200 mL for G-Rex®100M). In other words, the initial culture volume for the first expansion is 20% of the volume capacity of the cell culture container/system.

Day 9—Cell culture medium having a volume of 3-fold of the volume of the initial culture volume is added or exchanged.

Day 14—Pre-REP TIL harvest.

### Culture Condition (F-xiv):

Day 0—The initial culture volume is 25 mL for G-Rex®10M (or 250 mL for G-Rex®100M). In other words, the initial culture volume for the first expansion is 25% of the volume capacity of the cell culture container/system.

Day 9—Cell culture medium having a volume of 3-fold of the volume of the initial culture volume is added or exchanged.

Day 14—Pre-REP TIL harvest.

### Culture Condition (F-xv):

Day 0—The initial culture volume is 30 mL for G-Rex®10M (or 300 mL for G-Rex®100M). In other words, the initial culture volume for the first expansion is 30% of the volume capacity of the cell culture container/system.

Day 9—Cell culture medium having a volume of 3-fold of the volume of the initial culture volume is exchanged.

Day 14—Pre-REP TIL harvest.

### Culture Condition (G-i):

Day 0—The initial culture volume is 10 mL for G-Rex®10M (or 100 mL for G-Rex® 100M). In other words, the initial culture volume for the first expansion is 10% of the volume capacity of the cell culture container/system.

Day 10—A cell culture medium having a volume of 1-fold of the volume of the initial culture volume is added or exchanged.

Day 14—Pre-REP TIL harvest.

### Culture Condition (G-ii):

Day 0—The initial culture volume is 20 mL for G-Rex®10M (or 200 mL for G-Rex®100M). In other words, the initial culture volume for the first expansion is 20% of the volume capacity of the cell culture container/system.

Day 10—Cell culture medium having a volume of 1-fold of the volume of the initial culture volume is added or exchanged.

Day 14—Pre-REP TIL harvest.

### Culture Condition (G-iii):

Day 0—The initial culture volume is 25 mL for G-Rex®10M (or 250 mL for G-Rex®100M). In other words, the initial culture volume for the first expansion is 25% of the volume capacity of the cell culture container/system.

Day 10—Cell culture medium having a volume of I-fold of the volume of the initial culture volume is added or exchanged.

Day 14—Pre-REP TIL harvest.

### Culture Condition (G-iv):

Day 0—The initial culture volume is 30 mL for G-Rex®10M (or 300 mL for G-Rex® 100M). In other words, the initial culture volume for the first expansion is 30% of the volume capacity of the cell culture container/system.

Day 10—Cell culture medium having a volume of I-fold of the volume of the initial culture volume is added or exchanged.

Day 14—Pre-REP TIL harvest.

### Culture Condition (G-v):

Day 0—The initial culture volume is 50 mL for G-Rex®10M (or 500 mL for G-Rex®100M). In other words, the initial culture volume for the first expansion is 50% of the volume capacity of the cell culture container/system.

Day 10—Cell culture medium having a volume of 1-fold of the volume of the initial culture volume is added or exchanged.

Day 14—Pre-REP TIL harvest.

### Culture Condition (G-vi):

Day 0—The initial culture volume is 100 mL for G-Rex®10M (or 1000 mL for G-Rex®100M). In other words, the initial culture volume for the first expansion is 100% of the volume capacity of the cell culture container/system.

Day 10—Cell culture medium having a volume of I-fold of the volume of the initial culture volume is exchanged.

Day 14—Pre-REP TIL harvest.

### Culture Condition (G-vii):

Day 0—The initial culture volume is 10 mL for G-Rex®10M (or 100 mL for G-Rex®100M). In other words, the initial culture volume for the first expansion is 10% of the volume capacity of the cell culture container/system.

Day 10—A cell culture medium having a volume of 2-fold of the volume of the initial culture volume is added or exchanged.

Day 14—Pre-REP TIL harvest.

### Culture Condition (G-viii):

Day 0—The initial culture volume is 20 mL for G-Rex®10M (or 200 mL for G-Rex®100M). In other words, the initial culture volume for the first expansion is 20% of the volume capacity of the cell culture container/system.

Day 10—Cell culture medium having a volume of 2-fold of the volume of the initial culture volume is added or exchanged.

Day 14—Pre-REP TIL harvest.

### Culture Condition (G-ix):

Day 0—The initial culture volume is 25 mL for G-Rex®10M (or 250 mL for G-Rex®100M). In other words, the initial culture volume for the first expansion is 25% of the volume capacity of the cell culture container/system.

Day 10—Cell culture medium having a volume of 2-fold of the volume of the initial culture volume is added or exchanged.

Day 14—Pre-REP TIL harvest.

### Culture Condition (G-x):

Day 0—The initial culture volume is 30 mL for G-Rex®10M (or 300 mL for G-Rex®100M). In other words, the initial culture volume for the first expansion is 30% of the volume capacity of the cell culture container/system.

Day 10—Cell culture medium having a volume of 2-fold of the volume of the initial culture volume is added or exchanged.

Day 14—Pre-REP TIL harvest.

### Culture Condition (G-xi):

Day 0—The initial culture volume is 50 mL for G-Rex®10M (or 500 mL for G-Rex®100M). In other words, the initial culture volume for the first expansion is 50% of the volume capacity of the cell culture container/system.

Day 10—Cell culture medium having a volume of 2-fold of the volume of the initial culture volume is exchanged.

Day 14—Pre-REP TIL harvest.

### Culture Condition (G-xii):

Day 0—The initial culture volume is 10 mL for G-Rex®10M (or 100 mL for G-Rex®100M). In other words, the initial culture volume for the first expansion is 10% of the volume capacity of the cell culture container/system.

Day 10—A cell culture medium having a volume of 3-fold of the volume of the initial culture volume is added or exchanged.

Day 14—Pre-REP TIL harvest.

### Culture Condition (G-xiii):

Day 0—The initial culture volume is 20 mL for G-Rex®10M (or 200 mL for G-Rex®100M). In other words, the initial culture volume for the first expansion is 20% of the volume capacity of the cell culture container/system.

Day 10—Cell culture medium having a volume of 3-fold of the volume of the initial culture volume is added or exchanged.

Day 14—Pre-REP TIL harvest.

### Culture Condition (G-xiv):

Day 0—The initial culture volume is 25 mL for G-Rex®10M (or 250 mL for G-Rex®100M). In other words, the initial culture volume for the first expansion is 25% of the volume capacity of the cell culture container/system.

Day 10—Cell culture medium having a volume of 3-fold of the volume of the initial culture volume is added or exchanged.

Day 14—Pre-REP TIL harvest.

### Culture Condition (G-xv):

Day 0—The initial culture volume is 30 mL for G-Rex®10M (or 300 mL for G-Rex®100M). In other words, the initial culture volume for the first expansion is 30% of the volume capacity of the cell culture container/system.

Day 10—Cell culture medium having a volume of 3-fold of the volume of the initial culture volume is exchanged.

Day 14—Pre-REP TIL harvest.

### Culture Condition (H-i):

Day 0—The initial culture volume is 10 mL for G-Rex®10M (or 100 mL for G-Rex®100M). In other words, the initial culture volume for the first expansion is 10% of the volume capacity of the cell culture container/system.

Day 11—A cell culture medium having a volume of 1-fold of the volume of the initial culture volume is added or exchanged.

Day 14—Pre-REP TIL harvest.

### Culture Condition (H-ii):

Day 0—The initial culture volume is 20 mL for G-Rex®10M (or 200 mL for G-Rex®100M). In other words, the initial culture volume for the first expansion is 20% of the volume capacity of the cell culture container/system.

Day 11—Cell culture medium having a volume of I-fold of the volume of the initial culture volume is added or exchanged.

Day 14—Pre-REP TIL harvest.

### Culture Condition (H-iii):

Day 0—The initial culture volume is 25 mL for G-Rex®10M (or 250 mL for G-Rex®100M). In other words, the initial culture volume for the first expansion is 25% of the volume capacity of the cell culture container/system.

Day 11—Cell culture medium having a volume of 1-fold of the volume of the initial culture volume is added or exchanged.

Day 14—Pre-REP TIL harvest.

### Culture Condition (H-iv):

Day 0—The initial culture volume is 30 mL for G-Rex®10M (or 300 mL for G-Rex®100M). In other words, the initial culture volume for the first expansion is 30% of the volume capacity of the cell culture container/system.

Day 11—Cell culture medium having a volume of 1-fold of the volume of the initial culture volume is added or exchanged.

Day 14—Pre-REP TIL harvest.

### Culture Condition (H-v):

Day 0—The initial culture volume is 50 mL for G-Rex®10M (or 500 mL for G-Rex®100M). In other words, the initial culture volume for the first expansion is 50% of the volume capacity of the cell culture container/system.

Day 11—Cell culture medium having a volume of 1-fold of the volume of the initial culture volume is added or exchanged.

Day 14—Pre-REP TIL harvest.

### Culture Condition (H-vi):

Day 0—The initial culture volume is 100 mL for G-Rex®10M (or 1000 mL for G-Rex® 100M). In other words, the initial culture volume for the first expansion is 100% of the volume capacity of the cell culture container/system.

Day 11—Cell culture medium having a volume of I-fold of the volume of the initial culture volume is exchanged.

Day 14—Pre-REP TIL harvest.

### Culture Condition (H-vii):

Day 0—The initial culture volume is 10 mL for G-Rex®10M (or 100 mL for G-Rex®100M). In other words, the initial culture volume for the first expansion is 10% of the volume capacity of the cell culture container/system.

Day 11—A cell culture medium having a volume of 2-fold of the volume of the initial culture volume is added or exchanged.

Day 14—Pre-REP TIL harvest.

### Culture Condition (H-viii):

Day 0—The initial culture volume is 20 mL for G-Rex®10M (or 200 mL for G-Rex®100M). In other words, the initial culture volume for the first expansion is 20% of the volume capacity of the cell culture container/system.

Day 11—Cell culture medium having a volume of 2-fold of the volume of the initial culture volume is added or exchanged.

Day 14—Pre-REP TIL harvest.

### Culture Condition (H-ix):

Day 0—The initial culture volume is 25 mL for G-Rex®10M (or 250 mL for G-Rex®100M). In other words, the initial culture volume for the first expansion is 25% of the volume capacity of the cell culture container/system.

Day 11—Cell culture medium having a volume of 2-fold of the volume of the initial culture volume is added or exchanged.

Day 14—Pre-REP TIL harvest.

### Culture Condition (H-x):

Day 0—The initial culture volume is 30 mL for G-Rex®10M (or 300 mL for G-Rex®100M). In other words, the initial culture volume for the first expansion is 30% of the volume capacity of the cell culture container/system.

Day 11—Cell culture medium having a volume of 2-fold of the volume of the initial culture volume is added or exchanged.

Day 14—Pre-REP TIL harvest.

### Culture Condition (H-xi):

Day 0—The initial culture volume is 50 mL for G-Rex®10M (or 500 mL for G-Rex®100M). In other words, the initial culture volume for the first expansion is 50% of the volume capacity of the cell culture container/system.

Day 11—Cell culture medium having a volume of 2-fold of the volume of the initial culture volume is exchanged.

Day 14—Pre-REP TIL harvest.

### Culture Condition (H-xii):

Day 0—The initial culture volume is 10 mL for G-Rex®10M (or 100 mL for G-Rex®100M). In other words, the initial culture volume for the first expansion is 10% of the volume capacity of the cell culture container/system.

Day 11—A cell culture medium having a volume of 3-fold of the volume of the initial culture volume is added or exchanged.

Day 14—Pre-REP TIL harvest.

### Culture Condition (H-xiii):

Day 0—The initial culture volume is 20 mL for G-Rex®10M (or 200 mL for G-Rex® 100M). In other words, the initial culture volume for the first expansion is 20% of the volume capacity of the cell culture container/system.

Day 11—Cell culture medium having a volume of 3-fold of the volume of the initial culture volume is added or exchanged.

Day 14—Pre-REP TIL harvest.

### Culture Condition (H-xiv):

Day 0—The initial culture volume is 25 mL for G-Rex®10M (or 250 mL for G-Rex®100M). In other words, the initial culture volume for the first expansion is 25% of the volume capacity of the cell culture container/system.

Day 11—Cell culture medium having a volume of 3-fold of the volume of the initial culture volume is added or exchanged.

Day 14—Pre-REP TIL harvest.

### Culture Condition (H-xv):

Day 0—The initial culture volume is 30 mL for G-Rex®10M (or 300 mL for G-Rex®100M). In other words, the initial culture volume for the first expansion is 30% of the volume capacity of the cell culture container/system.

Day 11—Cell culture medium having a volume of 3-fold of the volume of the initial culture volume is exchanged.

Day 14—Pre-REP TIL harvest.

### Culture Condition (I-i):

Day 0—The initial culture volume is 10 mL for G-Rex®10M (or 100 mL for G-Rex® 100M). In other words, the initial culture volume for the first expansion is 10% of the volume capacity of the cell culture container/system.

Day 8—A cell culture medium having a volume of 1-fold of the volume of the initial culture volume is added or exchanged.

Day 15—Pre-REP TIL harvest.

### Culture Condition (I-ii):

Day 0—The initial culture volume is 20 mL for G-Rex®10M (or 200 mL for G-Rex®100M). In other words, the initial culture volume for the first expansion is 20% of the volume capacity of the cell culture container/system.

Day 8—Cell culture medium having a volume of 1-fold of the volume of the initial culture volume is added or exchanged.

Day 15—Pre-REP TIL harvest.

### Culture Condition (I-iii):

Day 0—The initial culture volume is 25 mL for G-Rex®10M (or 250 mL for G-Rex®100M). In other words, the initial culture volume for the first expansion is 25% of the volume capacity of the cell culture container/system.

Day 8—Cell culture medium having a volume of 1-fold of the volume of the initial culture volume is added or exchanged.

Day 15—Pre-REP TIL harvest.

### Culture Condition (I-iv):

Day 0—The initial culture volume is 30 mL for G-Rex®10M (or 300 mL for G-Rex®100M). In other words, the initial culture volume for the first expansion is 30% of the volume capacity of the cell culture container/system.

Day 8—Cell culture medium having a volume of 1-fold of the volume of the initial culture volume is added or exchanged.

Day 15—Pre-REP TIL harvest.

### Culture Condition (I-v):

Day 0—The initial culture volume is 50 mL for G-Rex®10M (or 500 mL for G-Rex®100M). In other words, the initial culture volume for the first expansion is 50% of the volume capacity of the cell culture container/system.

Day 8—Cell culture medium having a volume of 1-fold of the volume of the initial culture volume is added or exchanged.

Day 15—Pre-REP TIL harvest.

Culture Condition (1-vi):

Day 0—The initial culture volume is 100 mL for G-Rex®10M (or 1000 mL for G-Rex® 100M). In other words, the initial culture volume for the first expansion is 100% of the volume capacity of the cell culture container/system.

Day 8—Cell culture medium having a volume of 1-fold of the volume of the initial culture volume is exchanged.

Day 15—Pre-REP TIL harvest.

### Culture Condition (I-vii):

Day 0—The initial culture volume is 10 mL for G-Rex®10M (or 100 mL for G-Rex®100M). In other words, the initial culture volume for the first expansion is 10% of the volume capacity of the cell culture container/system.

Day 8—A cell culture medium having a volume of 2-fold of the volume of the initial culture volume is added or exchanged.

Day 15—Pre-REP TIL harvest.

### Culture Condition (I-viii):

Day 0—The initial culture volume is 20 mL for G-Rex®10M (or 200 mL for G-Rex®100M). In other words, the initial culture volume for the first expansion is 20% of the volume capacity of the cell culture container/system.

Day 8—Cell culture medium having a volume of 2-fold of the volume of the initial culture volume is added or exchanged.

Day 15—Pre-REP TIL harvest.

Culture Condition (1-ix):

Day 0—The initial culture volume is 25 mL for G-Rex®10M (or 250 mL for G-Rex®100M). In other words, the initial culture volume for the first expansion is 25% of the volume capacity of the cell culture container/system.

Day 8—Cell culture medium having a volume of 2-fold of the volume of the initial culture volume is added or exchanged.

Day 15—Pre-REP TIL harvest.

### Culture Condition (I-x):

Day 0—The initial culture volume is 30 mL for G-Rex®10M (or 300 mL for G-Rex®100M). In other words, the initial culture volume for the first expansion is 30% of the volume capacity of the cell culture container/system.

Day 8—Cell culture medium having a volume of 2-fold of the volume of the initial culture volume is added or exchanged.

Day 15—Pre-REP TIL harvest.

### Culture Condition (I-xi):

Day 0—The initial culture volume is 50 mL for G-Rex®10M (or 500 mL for G-Rex®100M). In other words, the initial culture volume for the first expansion is 50% of the volume capacity of the cell culture container/system.

Day 8—Cell culture medium having a volume of 2-fold of the volume of the initial culture volume is exchanged.

Day 15—Pre-REP TIL harvest.

### Culture Condition (I-xii):

Day 0—The initial culture volume is 10 mL for G-Rex®10M (or 100 mL for G-Rex®100M). In other words, the initial culture volume for the first expansion is 10% of the volume capacity of the cell culture container/system.

Day 8—A cell culture medium having a volume of 3-fold of the volume of the initial culture volume is added or exchanged.

Day 15—Pre-REP TIL harvest.

### Culture Condition (I-xiii):

Day 0—The initial culture volume is 20 mL for G-Rex®10M (or 200 mL for G-Rex®100M). In other words, the initial culture volume for the first expansion is 20% of the volume capacity of the cell culture container/system.

Day 8—Cell culture medium having a volume of 3-fold of the volume of the initial culture volume is added or exchanged.

Day 15—Pre-REP TIL harvest.

### Culture Condition (I-xiv):

Day 0—The initial culture volume is 25 mL for G-Rex®10M (or 250 mL for G-Rex®100M). In other words, the initial culture volume for the first expansion is 25% of the volume capacity of the cell culture container/system.

Day 8—Cell culture medium having a volume of 3-fold of the volume of the initial culture volume is added or exchanged.

Day 15—Pre-REP TIL harvest.

### Culture Condition (I-xv):

Day 0—The initial culture volume is 30 mL for G-Rex®10M (or 300 mL for G-Rex®100M). In other words, the initial culture volume for the first expansion is 30% of the volume capacity of the cell culture container/system.

Day 8—Cell culture medium having a volume of 3-fold of the volume of the initial culture volume is exchanged.

Day 15—Pre-REP TIL harvest.

### Culture Condition (J-i):

Day 0—The initial culture volume is 10 mL for G-Rex®10M (or 100 mL for G-Rex®100M). In other words, the initial culture volume for the first expansion is 10% of the volume capacity of the cell culture container/system.

Day 9—A cell culture medium having a volume of 1-fold of the volume of the initial culture volume is added or exchanged.

Day 15—Pre-REP TIL harvest.

### Culture Condition (J-ii):

Day 0—The initial culture volume is 20 mL for G-Rex®10M (or 200 mL for G-Rex®100M). In other words, the initial culture volume for the first expansion is 20% of the volume capacity of the cell culture container/system.

Day 9—Cell culture medium having a volume of 1-fold of the volume of the initial culture volume is added or exchanged.

Day 15—Pre-REP TIL harvest.

### Culture Condition (J-iii):

Day 0—The initial culture volume is 25 mL for G-Rex®10M (or 250 mL for G-Rex®100M). In other words, the initial culture volume for the first expansion is 25% of the volume capacity of the cell culture container/system.

Day 9—Cell culture medium having a volume of 1-fold of the volume of the initial culture volume is added or exchanged.

Day 15—Pre-REP TIL harvest.

### Culture Condition (J-iv):

Day 0—The initial culture volume is 30 mL for G-Rex®10M (or 300 mL for G-Rex®100M). In other words, the initial culture volume for the first expansion is 30% of the volume capacity of the cell culture container/system.

Day 9—Cell culture medium having a volume of 1-fold of the volume of the initial culture volume is added or exchanged.

Day 15—Pre-REP TIL harvest.

### Culture Condition (J-v):

Day 0—The initial culture volume is 50 mL for G-Rex®10M (or 500 mL for G-Rex®100M). In other words, the initial culture volume for the first expansion is 50% of the volume capacity of the cell culture container/system.

Day 9—Cell culture medium having a volume of 1-fold of the volume of the initial culture volume is added or exchanged.

Day 15—Pre-REP TIL harvest.

### Culture Condition (J-vi):

Day 0—The initial culture volume is 100 mL for G-Rex®10M (or 1000 mL for G-Rex®100M). In other words, the initial culture volume for the first expansion is 100% of the volume capacity of the cell culture container/system.

Day 9—Cell culture medium having a volume of 1-fold of the volume of the initial culture volume is exchanged.

Day 15—Pre-REP TIL harvest.

### Culture Condition (J-vii):

Day 0—The initial culture volume is 10 mL for G-Rex®10M (or 100 mL for G-Rex®100M). In other words, the initial culture volume for the first expansion is 10% of the volume capacity of the cell culture container/system.

Day 9—A cell culture medium having a volume of 2-fold of the volume of the initial culture volume is added or exchanged.

Day 15—Pre-REP TIL harvest.

### Culture Condition (J-viii):

Day 0—The initial culture volume is 20 mL for G-Rex®10M (or 200 mL for G-Rex®100M). In other words, the initial culture volume for the first expansion is 20% of the volume capacity of the cell culture container/system.

Day 9—Cell culture medium having a volume of 2-fold of the volume of the initial culture volume is added or exchanged.

Day 15—Pre-REP TIL harvest.

### Culture Condition (J-ix):

Day 0—The initial culture volume is 25 mL for G-Rex®10M (or 250 mL for G-Rex®100M). In other words, the initial culture volume for the first expansion is 25% of the volume capacity of the cell culture container/system.

Day 9—Cell culture medium having a volume of 2-fold of the volume of the initial culture volume is added or exchanged.

Day 15—Pre-REP TIL harvest.

### Culture Condition (J-x):

Day 0—The initial culture volume is 30 mL for G-Rex®10M (or 300 mL for G-Rex®100M). In other words, the initial culture volume for the first expansion is 30% of the volume capacity of the cell culture container/system.

Day 9—Cell culture medium having a volume of 2-fold of the volume of the initial culture volume is added or exchanged.

Day 15—Pre-REP TIL harvest.

### Culture Condition (J-xi):

Day 0—The initial culture volume is 50 mL for G-Rex®10M (or 500 mL for G-Rex®100M). In other words, the initial culture volume for the first expansion is 50% of the volume capacity of the cell culture container/system.

Day 9—Cell culture medium having a volume of 2-fold of the volume of the initial culture volume is exchanged.

Day 15—Pre-REP TIL harvest.

### Culture Condition (J-xii):

Day 0—The initial culture volume is 10 mL for G-Rex®10M (or 100 mL for G-Rex®100M). In other words, the initial culture volume for the first expansion is 10% of the volume capacity of the cell culture container/system.

Day 9—A cell culture medium having a volume of 3-fold of the volume of the initial culture volume is added or exchanged.

Day 15—Pre-REP TIL harvest.

### Culture Condition (J-xiii):

Day 0—The initial culture volume is 20 mL for G-Rex®10M (or 200 mL for G-Rex®100M). In other words, the initial culture volume for the first expansion is 20% of the volume capacity of the cell culture container/system.

Day 9—Cell culture medium having a volume of 3-fold of the volume of the initial culture volume is added or exchanged.

Day 15—Pre-REP TIL harvest.

### Culture Condition (J-xiv):

Day 0—The initial culture volume is 25 mL for G-Rex®10M (or 250 mL for G-Rex®100M). In other words, the initial culture volume for the first expansion is 25% of the volume capacity of the cell culture container/system.

Day 9—Cell culture medium having a volume of 3-fold of the volume of the initial culture volume is added or exchanged.

Day 15—Pre-REP TIL harvest.

### Culture Condition (J-xv):

Day 0—The initial culture volume is 30 mL for G-Rex®10M (or 300 mL for G-Rex® 100M). In other words, the initial culture volume for the first expansion is 30% of the volume capacity of the cell culture container/system.

Day 9—Cell culture medium having a volume of 3-fold of the volume of the initial culture volume is exchanged.

Day 15—Pre-REP TIL harvest.

### Culture Condition (K-i):

Day 0—The initial culture volume is 10 mL for G-Rex®10M (or 100 mL for G-Rex® 100M). In other words, the initial culture volume for the first expansion is 10% of the volume capacity of the cell culture container/system.

Day 10—A cell culture medium having a volume of 1-fold of the volume of the initial culture volume is added or exchanged.

Day 15—Pre-REP TIL harvest.

### Culture Condition (K-ii):

Day 0—The initial culture volume is 20 mL for G-Rex®10M (or 200 mL for G-Rex®100M). In other words, the initial culture volume for the first expansion is 20% of the volume capacity of the cell culture container/system.

Day 10—Cell culture medium having a volume of 1-fold of the volume of the initial culture volume is added or exchanged.

Day 15—Pre-REP TIL harvest.

### Culture Condition (K-iii):

Day 0—The initial culture volume is 25 mL for G-Rex®10M (or 250 mL for G-Rex®100M). In other words, the initial culture volume for the first expansion is 25% of the volume capacity of the cell culture container/system.

Day 10—Cell culture medium having a volume of I-fold of the volume of the initial culture volume is added or exchanged.

Day 15—Pre-REP TIL harvest.

### Culture Condition (K-iv):

Day 0—The initial culture volume is 30 mL for G-Rex®10M (or 300 mL for G-Rex®100M). In other words, the initial culture volume for the first expansion is 30% of the volume capacity of the cell culture container/system.

Day 10—Cell culture medium having a volume of I-fold of the volume of the initial culture volume is added or exchanged.

Day 15—Pre-REP TIL harvest.

### Culture Condition (K-v):

Day 0—The initial culture volume is 50 mL for G-Rex®10M (or 500 mL for G-Rex®100M). In other words, the initial culture volume for the first expansion is 50% of the volume capacity of the cell culture container/system.

Day 10—Cell culture medium having a volume of I-fold of the volume of the initial culture volume is added or exchanged.

Day 15—Pre-REP TIL harvest.

### Culture Condition (K-vi):

Day 0—The initial culture volume is 100 mL for G-Rex®10M (or 1000 mL for G-Rex®100M). In other words, the initial culture volume for the first expansion is 100% of the volume capacity of the cell culture container/system.

Day 10—Cell culture medium having a volume of 1-fold of the volume of the initial culture volume is exchanged.

Day 15—Pre-REP TIL harvest.

### Culture Condition (K-vii):

Day 0—The initial culture volume is 10 mL for G-Rex®10M (or 100 mL for G-Rex®100M). In other words, the initial culture volume for the first expansion is 10% of the volume capacity of the cell culture container/system.

Day 10—A cell culture medium having a volume of 2-fold of the volume of the initial culture volume is added or exchanged.

Day 15—Pre-REP TIL harvest.

### Culture Condition (K-viii):

Day 0—The initial culture volume is 20 mL for G-Rex®10M (or 200 mL for G-Rex®100M). In other words, the initial culture volume for the first expansion is 20% of the volume capacity of the cell culture container/system.

Day 10—Cell culture medium having a volume of 2-fold of the volume of the initial culture volume is added or exchanged.

Day 15—Pre-REP TIL harvest.

### Culture Condition (K-ix):

Day 0—The initial culture volume is 25 mL for G-Rex®10M (or 250 mL for G-Rex®100M). In other words, the initial culture volume for the first expansion is 25% of the volume capacity of the cell culture container/system.

Day 10—Cell culture medium having a volume of 2-fold of the volume of the initial culture volume is added or exchanged.

Day 15—Pre-REP TIL harvest.

### Culture Condition (K-x):

Day 0—The initial culture volume is 30 mL for G-Rex®10M (or 300 mL for G-Rex®100M). In other words, the initial culture volume for the first expansion is 30% of the volume capacity of the cell culture container/system.

Day 10—Cell culture medium having a volume of 2-fold of the volume of the initial culture volume is added or exchanged.

Day 15—Pre-REP TIL harvest.

### Culture Condition (K-xi):

Day 0—The initial culture volume is 50 mL for G-Rex®10M (or 500 mL for G-Rex®100M). In other words, the initial culture volume for the first expansion is 50% of the volume capacity of the cell culture container/system.

Day 10—Cell culture medium having a volume of 2-fold of the volume of the initial culture volume is exchanged.

Day 15—Pre-REP TIL harvest.

### Culture Condition (K-xii):

Day 0—The initial culture volume is 10 mL for G-Rex®10M (or 100 mL for G-Rex®100M). In other words, the initial culture volume for the first expansion is 10% of the volume capacity of the cell culture container/system.

Day 10—A cell culture medium having a volume of 3-fold of the volume of the initial culture volume is added or exchanged.

Day 15—Pre-REP TIL harvest.

### Culture Condition (K-xiii):

Day 0—The initial culture volume is 20 mL for G-Rex®10M (or 200 mL for G-Rex®100M). In other words, the initial culture volume for the first expansion is 20% of the volume capacity of the cell culture container/system.

Day 10—Cell culture medium having a volume of 3-fold of the volume of the initial culture volume is added or exchanged.

Day 15—Pre-REP TIL harvest.

### Culture Condition (K-xiv):

Day 0—The initial culture volume is 25 mL for G-Rex®10M (or 250 mL for G-Rex®100M). In other words, the initial culture volume for the first expansion is 25% of the volume capacity of the cell culture container/system.

Day 10—Cell culture medium having a volume of 3-fold of the volume of the initial culture volume is added or exchanged.

Day 15—Pre-REP TIL harvest.

### Culture Condition (K-xv):

Day 0—The initial culture volume is 30 mL for G-Rex®10M (or 300 mL for G-Rex®100M). In other words, the initial culture volume for the first expansion is 30% of the volume capacity of the cell culture container/system.

Day 10—Cell culture medium having a volume of 3-fold of the volume of the initial culture volume is exchanged.

Day 15—Pre-REP TIL harvest.

### Culture Condition (L-i):

Day 0—The initial culture volume is 10 mL for G-Rex®10M (or 100 mL for G-Rex®100M). In other words, the initial culture volume for the first expansion is 10% of the volume capacity of the cell culture container/system.

Day 11—A cell culture medium having a volume of 1-fold of the volume of the initial culture volume is added or exchanged.

Day 15—Pre-REP TIL harvest.

### Culture Condition (L-ii):

Day 0—The initial culture volume is 20 mL for G-Rex®10M (or 200 mL for G-Rex®100M). In other words, the initial culture volume for the first expansion is 20% of the volume capacity of the cell culture container/system.

Day 11—Cell culture medium having a volume of I-fold of the volume of the initial culture volume is added or exchanged.

Day 15—Pre-REP TIL harvest.

### Culture Condition (L-iii):

Day 0—The initial culture volume is 25 mL for G-Rex®10M (or 250 mL for G-Rex®100M). In other words, the initial culture volume for the first expansion is 25% of the volume capacity of the cell culture container/system.

Day 11—Cell culture medium having a volume of 1-fold of the volume of the initial culture volume is added or exchanged.

Day 15—Pre-REP TIL harvest.

### Culture Condition (L-iv):

Day 0—The initial culture volume is 30 mL for G-Rex®10M (or 300 mL for G-Rex®100M). In other words, the initial culture volume for the first expansion is 30% of the volume capacity of the cell culture container/system.

Day 11—Cell culture medium having a volume of I-fold of the volume of the initial culture volume is added or exchanged.

Day 15—Pre-REP TIL harvest.

### Culture Condition (L-v):

Day 0—The initial culture volume is 50 mL for G-Rex®10M (or 500 mL for G-Rex®100M). In other words, the initial culture volume for the first expansion is 50% of the volume capacity of the cell culture container/system.

Day 11—Cell culture medium having a volume of I-fold of the volume of the initial culture volume is added or exchanged.

Day 15—Pre-REP TIL harvest.

### Culture Condition (L-vi):

Day 0—The initial culture volume is 100 mL for G-Rex®10M (or 1000 mL for G-Rex®100M). In other words, the initial culture volume for the first expansion is 100% of the volume capacity of the cell culture container/system.

Day 11—Cell culture medium having a volume of 1-fold of the volume of the initial culture volume is exchanged.

Day 15—Pre-REP TIL harvest.

### Culture Condition (L-vii):

Day 0—The initial culture volume is 10 mL for G-Rex®10M (or 100 mL for G-Rex®100M). In other words, the initial culture volume for the first expansion is 10% of the volume capacity of the cell culture container/system.

Day 11—A cell culture medium having a volume of 2-fold of the volume of the initial culture volume is added or exchanged.

Day 15—Pre-REP TIL harvest.

### Culture Condition (L-viii):

Day 0—The initial culture volume is 20 mL for G-Rex®10M (or 200 mL for G-Rex®100M). In other words, the initial culture volume for the first expansion is 20% of the volume capacity of the cell culture container/system.

Day 11—Cell culture medium having a volume of 2-fold of the volume of the initial culture volume is added or exchanged.

Day 15—Pre-REP TIL harvest.

### Culture Condition (L-ix):

Day 0—The initial culture volume is 25 mL for G-Rex®10M (or 250 mL for G-Rex®100M). In other words, the initial culture volume for the first expansion is 25% of the volume capacity of the cell culture container/system.

Day 11—Cell culture medium having a volume of 2-fold of the volume of the initial culture volume is added or exchanged.

Day 15—Pre-REP TIL harvest.

### Culture Condition (L-x):

Day 0—The initial culture volume is 30 mL for G-Rex®10M (or 300 mL for G-Rex®100M). In other words, the initial culture volume for the first expansion is 30% of the volume capacity of the cell culture container/system.

Day 11—Cell culture medium having a volume of 2-fold of the volume of the initial culture volume is added or exchanged.

Day 15—Pre-REP TIL harvest.

### Culture Condition (L-xi):

Day 0—The initial culture volume is 50 mL for G-Rex®10M (or 500 mL for G-Rex®100M). In other words, the initial culture volume for the first expansion is 50% of the volume capacity of the cell culture container/system.

Day 11—Cell culture medium having a volume of 2-fold of the volume of the initial culture volume is exchanged.

Day 15—Pre-REP TIL harvest.

### Culture Condition (L-xii):

Day 0—The initial culture volume is 10 mL for G-Rex®10M (or 100 mL for G-Rex®100M). In other words, the initial culture volume for the first expansion is 10% of the volume capacity of the cell culture container/system.

Day 11—A cell culture medium having a volume of 3-fold of the volume of the initial culture volume is added or exchanged.

Day 15—Pre-REP TIL harvest.

### Culture Condition (L-xiii):

Day 0—The initial culture volume is 20 mL for G-Rex®10M (or 200 mL for G-Rex®100M). In other words, the initial culture volume for the first expansion is 20% of the volume capacity of the cell culture container/system.

Day 11—Cell culture medium having a volume of 3-fold of the volume of the initial culture volume is added or exchanged.

Day 15—Pre-REP TIL harvest.

### Culture Condition (L-xiv):

Day 0—The initial culture volume is 25 mL for G-Rex®10M (or 250 mL for G-Rex®100M). In other words, the initial culture volume for the first expansion is 25% of the volume capacity of the cell culture container/system.

Day 11—Cell culture medium having a volume of 3-fold of the volume of the initial culture volume is added or exchanged.

Day 15—Pre-REP TIL harvest.

### Culture Condition (L-xv):

Day 0—The initial culture volume is 30 mL for G-Rex®10M (or 300 mL for G-Rex®100M). In other words, the initial culture volume for the first expansion is 30% of the volume capacity of the cell culture container/system.

Day 11—Cell culture medium having a volume of 3-fold of the volume of the initial culture volume is exchanged.

Day 15—Pre-REP TIL harvest.

Other suitable culture conditions may also be used.

### Success Rate Before and After Optimization

Before optimization, the success rate of pre-REP TIL production was low with an average yield of 3.10×10⁶ cells/fragment (Table 1). After the pre-REP procedure was optimized, the full-scale culture in G-Rex® 100M devices was performed, which resulted in an average yield of 16.29×10⁶ cells/fragment (Table 2). Therefore, our optimized procedure has led to a significantly higher yield of harvested pre-REP TILs than before optimization (P=0.00125; n=12 for before optimization runs and n=8 for after optimization runs) (Table 2).

TABLE 1
Yields of pre-REP culture before optimization

| | | | No. of cells |
|---|---|---|---|
| | | Total pre-REP | (×10⁶) per |
| Sample ID | Device | yield (×10⁶) | fragment |
| T5101001 | G-Rex ®10M | 40.30 | 8.06 |
| T5101002 | G-Rex ®10M | 17.30 | 3.46 |
| T5101004 | G-Rex ®10M | 1.16 | 0.23 |
| T5101005 | G-Rex ®10M | 51.40 | 10.34 |
| T5101006 | G-Rex ®10M | 0.17 | 0.03 |
| T5101007 | G-Rex ®10M | 1.18 | 0.24 |
| T5101008 | G-Rex ®10M | 30.43 | 6.09 |
| T5101009a | G-Rex ®10M | 2.79 | 0.56 |
| T5101010a | G-Rex ®10M | 14.49 | 2.90 |
| T5101009b | G-Rex ®100M | 2.20 | 0.22 |
| T5101010b | G-Rex ®100M | 93.00 | 4.65 |
| T5101013 | G-Rex ®100M | 6.20 | 0.39 |
| Mean ± S.D. | | 21.72 ± 28.19 | 3.10 ± 3.52 |
| 95% CI | | (3.80; 39.63) | (0.86; 5.33) |

S.D., standard deviation; CI, confidence interval.

TABLE 2
Yields of pre-REP culture after optimization

| | | | No. of cells |
|---|---|---|---|
| | | Total pre-REP | (×10⁶) per |
| Sample ID | Device | yield (×10⁶) | fragment |
| T5101014 | G-Rex ®100M | 783 | 34.04 |
| T5101015 | G-Rex ®100M | 459 | 13.50 |
| T5101016 | G-Rex ®100M | 148 | 14.80 |
| T5101017 | G-Rex ®100M | 275 | 21.15 |
| T5101018 | G-Rex ®100M | 216 | 12.70 |
| T5101021 | G-Rex ®100M | 67 | 3.72 |
| T5101022 | G-Rex ®100M | 90 | 3.75 |
| T5101024 | G-Rex ®100M | 693 | 26.65 |
| Mean ± S.D. | | 341.4 ± 274.8 | 16.29 ± 10.58 |
| 95% CI | | (111.7; 571.1) | (7.44; 25.14) |

S.D., standard deviation; CI, confidence interval.

### Evaluation of Glucose and Lactate Concentrations in the Standardized Pre-REP Protocol

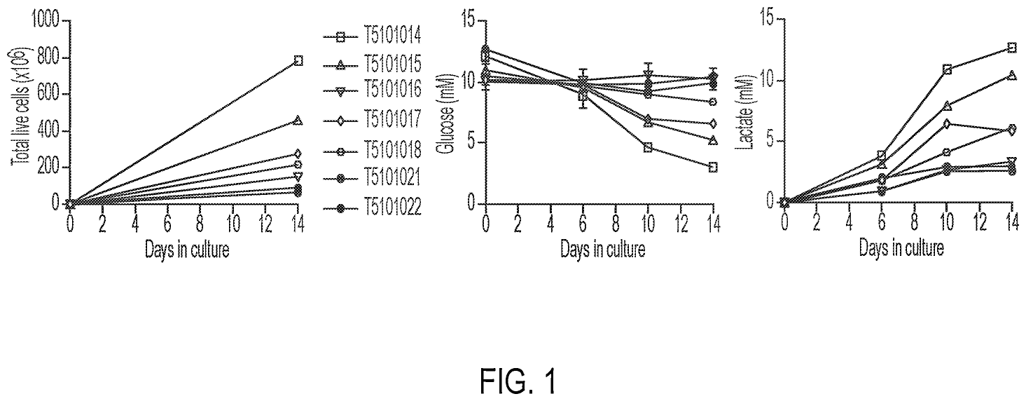

To validate that the media addition in the standardized pre-REP protocol is sufficient for the process, we collected supernatants on Days 0, 6, 10, and 14 from 7 runs (T5101014-T5101018, T5101021 and T5101022) and evaluated final TIL yield, glucose and lactate concentrations. By Day 6 and thereafter, glucose and lactate levels changed proportionally to the degree of TIL expansion (FIG. 1). However, the glucose levels at harvest remained high enough (7.69±2.86 mM; 95% CI, 5.05-10.33 mM; n=7) to support T-cell growth (Table 3). Importantly, lactate levels (6.30±3.92 mM; 95% CI, 2.67-9.93 mM; n=7) stayed well below 20 mM, a level above which CD8⁺ killer T cells have been shown to exhibit reduced proliferation, viability, and function¹⁴. Taken together, our results showed that the optimized pre-REP process could sustain glucose and lactate levels within a range that is supportive of TIL migration and expansion.

TABLE 3
Summary of the numbers of seeded tumor fragments,
glucose and lactate levels, and TIL yields in cultures
following the standardized pre-REP protocol

| | Day 14 | Day 14 | Pre-REP TIL yield |
|---|---|---|---|
| Sample ID | Glucose (mM) | Lactate (mM) | on Day 14 (×10⁶) |
| T5101014 | 3.0 | 12.7 | 783 |
| T5101015 | 5.2 | 10.5 | 459 |
| T5101016 | 10.3 | 3.3 | 148 |
| T5101017 | 6.6 | 5.9 | 275 |
| T5101018 | 8.4 | 6.1 | 216 |
| T5101021 | 10.4 | 2.6 | 67 |
| T5101022 | 9.9 | 3.0 | 90 |
| Mean ± S.D. | 7.69 ± 2.86 | 6.30 ± 3.92 | 291.1 ± 254.0 |
| 95% CI | (5.05; 10.33) | (2.67; 9.93) | (56.20; 526.1) |

S.D., standard deviation; CI, confidence interval.

### Pre-REP TIL Characterization

As expected, pre-REP TILs expanded under optimized conditions had a high viability and high percentages of CD3⁺ cells. The average viability of pre-REP TILs was 93.68%±2.48% (n=8). The average percentage of CD3⁺ cells was 89.43%±5.31% (n=8).

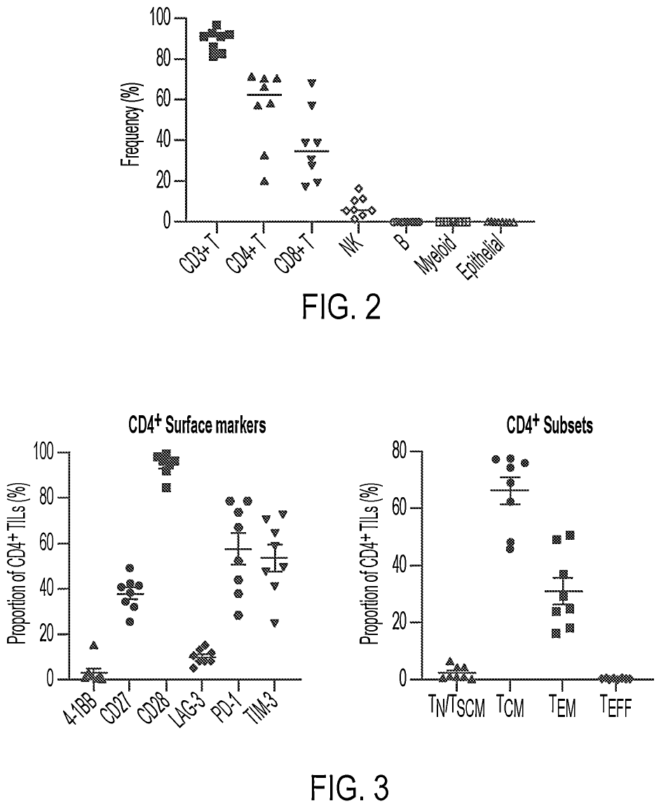

The proportion of CD8⁺ cells in the pre-REP TIL products varied across donors, and the average percentage (37.35%) was similar to other NSCLC pre-REP TIL products³. The pre-REP material had only low percentages of NK cells, B cells, myeloid cells, and epithelial cells. The percentages of NK cells, B cells, myeloid cells and epithelial cells were 7.74%±4.87%, 0.118% 0.117%, 0.034%±0.032% and 0.074%±0.137%, respectively (n=8; FIG. 2 and Table 4).

TABLE 4
Summary of viability (%) and lineage distributions (%) of
harvested pre-REP TILs following the standardized protocol

| | | | CD3⁺/ | CD3⁺/ | | | | |
|---|---|---|---|---|---|---|---|---|
| Sample ID | Viability | CD3⁺ | CD4⁺ | CD8⁺ | NK | B | Myeloid | Epithelial |
| T5101014 | 92.1 | 83.00 | 33.20 | 57.10 | 11.70 | 0.340 | 0.027 | 0.025 |
| T5101015 | 94.3 | 85.90 | 58.30 | 39.00 | 10.70 | 0.140 | 0.035 | 0.070 |
| T5101016 | 92.4 | 91.20 | 57.60 | 38.90 | 5.86 | 0.230 | 0.033 | 0.014 |
| T5101017 | 90.4 | 81.80 | 70.90 | 17.30 | 16.50 | 0.015 | 0.110 | 0.029 |
| T5101018 | 95.7 | 91.10 | 66.90 | 30.90 | 6.20 | 0.002 | 0.019 | 0.023 |
| T5101021 | 91.2 | 93.20 | 71.70 | 19.50 | 3.58 | 0.120 | 0.019 | 0.019 |
| T5101022 | 96.6 | 92.20 | 70.50 | 27.70 | 5.77 | 0.062 | 0.014 | 0.410 |
| T5101024 | 96.7 | 97.00 | 20.20 | 68.40 | 1.58 | 0.035 | 0.011 | 0.004 |
| Mean | 93.68 | 89.43 | 56.16 | 37.35 | 7.74 | 0.118 | 0.034 | 0.074 |
| S.D. | 2.48 | 5.31 | 19.30 | 17.79 | 4.87 | 0.117 | 0.032 | 0.137 |
| Lower | 91.60 | 84.99 | 40.03 | 22.48 | 3.66 | 0.020 | 0.007 | −0.040 |
| 95% CI | | | | | | | | |
| Upper | 95.75 | 93.86 | 72.30 | 52.22 | 11.81 | 0.220 | 0.060 | 0.189 |
| 95% CI | | | | | | | | |

S.D., standard deviation; CI, confidence interval; CD3⁺/CD4⁺, the percentage of CD4⁺ cells among CD3⁺ cells; CD3⁺/CD8⁺, the percentage of CD8⁺ cells among CD3⁺ cells.

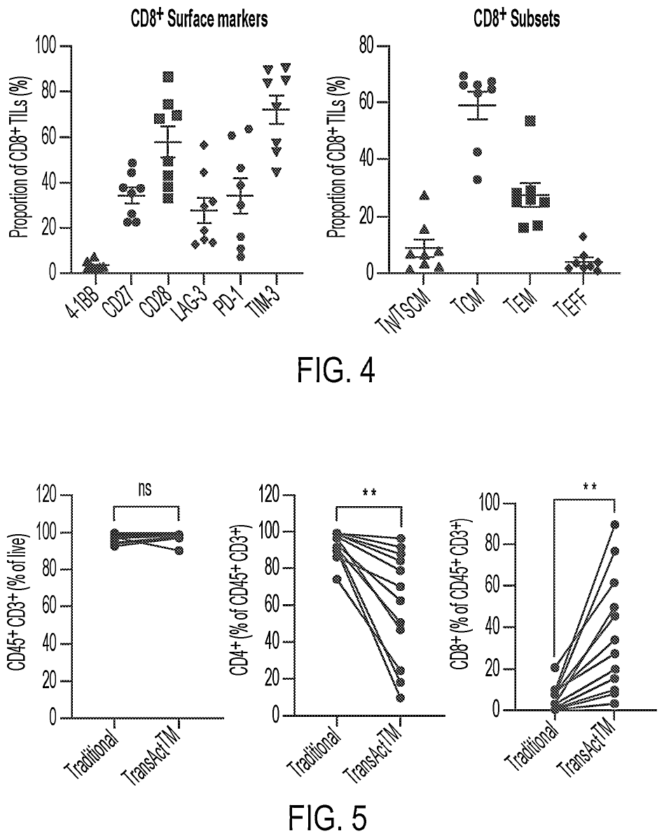

We also extensively analyzed the phenotypes of our pre-REP TILs by examining the expression of different activation/inhibition markers and the distributions of T-cell subsets. The data showed that these pre-REP TILs were activated with high expression of CD27 and CD28 with the positivity frequencies of 37.94%±7.17% and 94.51%±4.72%, respectively, among CD4⁺ pre-REP TILs (n=8, FIG. 3, Table 5), and 34.45%±9.62% and 57.66%±19.19%, respectively, among CD8_pre-REP TILs (n=8; FIG. 4, Table 7). Different inhibitory markers were expressed on CD4⁺ and CD8⁺ pre-REP T cells, with the lowest expression of LAG-3 on CD4⁺ pre-REP T cells with the positivity frequency of 10.07%±3.23% (n=8; FIG. 3, Table 5). PD-1 was moderately expressed on both CD4⁺ and CD8⁺ pre-REP T cells. The positivity frequencies of PD-1 were 57.43%±19.58% and 34.31%±21.67% in CD4⁺ and CD8_pre-REP T cells, respectively. TIM-3 was highly expressed on most CD8⁺ pre-REP TILs. The positivity frequencies of TIM-3 were 53.50%±16.12% in CD4⁺ (n=8, FIG. 3, Table 5) and 71.93%±17.85% in CD8⁺ pre-REP T cells (n=8; FIG. 4, Table 7).

One favorable trait of our pre-REP TILs was the high proportion of T_{CM} and reduced proportion of T_{EM} in both CD4⁺ and CD8′ pre-REP TILs. The percentages of T_{CM} were 66.28%±12.87% among CD4⁺ pre-REP TILs, and 59.04%±13.47% among CD8⁺ pre-REP TILs. The percentages of T_{EM} were 30.98%±13.25% in CD4⁺ (n=8; FIG. 3, Table 6) and 27.66%±11.59% in CD8⁺ pre-REP TILs (n=8; FIG. 4, Table 8). Moreover, the minimal proportions of the T_{EFF} subset (0.26%±0.19% and 4.27%±3.85% among CD4⁺ and CD8⁺ cells, respectively; n=8) were identified. In addition, the percentages of T_{N}/T_{SCM} subsets were also high in the harvested products: 2.49%±2.28% among CD4⁺ pre-REP TILs (n=8) and 9.04%±8.65% among CD8⁺ pre-REP TILs (n=8) (FIG. 3, Table 6; and FIG. 4, Table 8), indicating a prominent early memory TIL phenotype. This is of great importance, because the clinical benefit of TIL therapy is greatly dependent on the specific quality of TIL products, which are generated from pre-REP TILs. Overall, we have shown here a method to produce pre-REP TILs in an effective and timely manner, that results in a high yield and favorable TIL phenotype suggesting successful REP and associated with clinical efficacy.

TABLE 5
The positivity frequencies (%) of activation/inhibitory
surface markers among CD4⁺ pre-REP TILs

| Sample ID | 4-1BB | CD27 | CD28 | LAG-3 | PD-1 | TIM-3 |
|---|---|---|---|---|---|---|
| T5101014 | 1.67 | 38.50 | 84.20 | 5.38 | 28.20 | 72.20 |
| T5101015 | 1.33 | 42.10 | 96.20 | 8.29 | 73.50 | 64.10 |
| T5101016 | 2.27 | 32.00 | 96.10 | 15.30 | 52.00 | 41.00 |
| T5101017 | 0.32 | 34.30 | 99.10 | 7.99 | 78.60 | 47.60 |
| T5101018 | 0.65 | 40.80 | 97.90 | 13.10 | 43.90 | 70.30 |
| T5101021 | 0.98 | 25.60 | 95.00 | 10.70 | 66.90 | 49.60 |
| T5101022 | 3.05 | 41.20 | 96.10 | 8.08 | 78.40 | 24.50 |
| T5101024 | 15.4 | 49.00 | 91.50 | 11.70 | 37.90 | 58.70 |
| Mean | 3.21 | 37.94 | 94.51 | 10.07 | 57.43 | 53.50 |
| S.D. | 5.00 | 7.17 | 4.72 | 3.23 | 19.58 | 16.12 |
| Lower | −0.97 | 31.95 | 90.56 | 7.37 | 41.05 | 40.02 |
| 95% CI | | | | | | |
| Upper | 7.39 | 43.93 | 98.46 | 12.77 | 73.80 | 66.98 |
| 95% CI | | | | | | |

S.D., standard deviation; CI, confidence interval.

TABLE 6
The frequencies (%) of subsets among CD4⁺ pre-REP TILs

| Sample ID | T_{N}/T_{SCM} | T_{CM} | T_{EM} | T_{EFF} |
|---|---|---|---|---|
| T5101014 | 4.41 | 77.40 | 18.10 | 0.18 |
| T5101015 | 6.43 | 77.10 | 16.10 | 0.40 |
| T5101016 | 4.42 | 45.90 | 49.10 | 0.57 |
| T5101017 | 1.10 | 62.30 | 36.40 | 0.19 |
| T5101018 | 0.30 | 75.80 | 23.80 | 0.03 |
| T5101021 | 0.80 | 48.30 | 50.70 | 0.18 |
| T5101022 | 0.74 | 74.40 | 24.80 | 0.11 |
| T5101024 | 1.69 | 69.00 | 28.80 | 0.46 |
| Mean | 2.49 | 66.28 | 30.98 | 0.26 |
| S.D. | 2.28 | 12.87 | 13.25 | 0.19 |
| Lower | 0.58 | 55.52 | 19.90 | 0.11 |
| 95% CI | | | | |
| Upper | 4.39 | 77.03 | 42.05 | 0.42 |
| 95% CI | | | | |

S.D., standard deviation; CI, confidence interval; T_{N}, naive T cells; T_{SCM}, stem memory T cells; T_{CM}, central memory T cells; T_{EM}, effector memory T cells; T_{EFF}, effector T cells.

TABLE 7
The positivity frequencies (%) of activation/inhibitory
surface markers among CD8⁺ pre-REP TILs

| Sample ID | 4-1BB | CD27 | CD28 | LAG-3 | PD-1 | TIM-3 |
|---|---|---|---|---|---|---|
| T5101014 | 2.50 | 44.40 | 38.00 | 31.90 | 11.20 | 83.40 |
| T5101015 | 2.76 | 22.80 | 69.40 | 13.10 | 7.77 | 44.50 |
| T5101016 | 3.10 | 37.40 | 49.40 | 29.40 | 38.80 | 73.00 |
| T5101017 | 2.01 | 23.10 | 33.40 | 56.30 | 60.70 | 90.20 |
| T5101018 | 4.29 | 37.50 | 74.40 | 44.40 | 30.10 | 89.20 |
| T5101021 | 1.37 | 48.60 | 67.80 | 18.70 | 63.30 | 53.30 |
| T5101022 | 7.57 | 35.40 | 85.90 | 15.10 | 46.40 | 57.40 |
| T5101024 | 5.29 | 26.40 | 43.00 | 13.90 | 16.20 | 84.40 |
| Mean | 3.61 | 34.45 | 57.66 | 27.85 | 34.31 | 71.93 |
| S.D. | 2.03 | 9.62 | 19.19 | 15.86 | 21.67 | 17.85 |
| Lower | 1.92 | 26.41 | 41.62 | 14.59 | 16.19 | 57.00 |
| 95% CI | | | | | | |
| Upper | 5.31 | 42.49 | 73.70 | 41.11 | 52.43 | 86.85 |
| 95% CI | | | | | | |

S.D., standard deviation; CI, confidence interval.

TABLE 8
The frequencies (%) of subsets among CD8⁺ pre-REP TILs

| Sample ID | T_{N}/T_{SCM} | T_{CM} | T_{EM} | T_{EFF} |
|---|---|---|---|---|
| T5101014 | 15.70 | 66.10 | 16.20 | 2.07 |
| T5101015 | 7.89 | 63.30 | 25.10 | 3.71 |
| T5101016 | 27.50 | 42.60 | 17.00 | 12.90 |
| T5101017 | 6.85 | 33.00 | 53.70 | 6.41 |
| T5101018 | 3.59 | 66.10 | 29.30 | 0.99 |
| T5101021 | 2.37 | 67.30 | 28.40 | 1.93 |
| T5101022 | 1.85 | 69.20 | 25.50 | 3.43 |
| T5101024 | 6.55 | 64.70 | 26.10 | 2.72 |
| Mean | 9.04 | 59.04 | 27.66 | 4.27 |
| S.D. | 8.65 | 13.47 | 11.59 | 3.85 |
| Lower | 1.81 | 47.78 | 17.97 | 1.06 |
| 95% CI | | | | |
| Upper | 16.27 | 70.30 | 37.35 | 7.49 |
| 95% CI | | | | |

S.D., standard deviation; CI, confidence interval; T_{N}, naive T cells; T_{SCM}, stem memory T cells; T_{CM}, central memory T cells; T_{EM}, effector memory T cells; T_{EFF}, effector T cells.

### CONCLUSIONS

We have optimized a pre-REP TIL culture process, resulting in a high proportion of T_{CM} and healthy TIL phenotypes. Overall, these studies can be used to support pre-REP clinical manufacturing and subsequent TIL experiments.

### REFERENCES

1. Jansen, C. S. et al. An intra-tumoral niche maintains and differentiates stem-like CD8 T cells. Nature 576, 465-470 (2019).
2. Jin, J. et al. Simplified method of the growth of human tumor infiltrating lymphocytes in gas-permeable flasks to numbers needed for patient treatment. J Immunother 35, 283-292 (2012).
3. Creelan, B. C. et al. Tumor-infiltrating lymphocyte treatment for anti-PD-1-resistant metastatic lung cancer: a phase 1 trial. Nat Med 27, 1410-1418 (2021).
4. Rosenberg, S. A. et al. Durable complete responses in heavily pretreated patients with metastatic melanoma using T-cell transfer immunotherapy. Clin Cancer Res 17, 4550-4557 (2011).
5. Ando, M., Ito, M., Srirat, T., Kondo, T. & Yoshimura, A. Memory T cell, exhaustion, and tumor immunity. Inmunol Med 43, 1-9 (2020).
6. Rosenberg, S. A., Spiess, P. & Lafreniere, R. A new approach to the adoptive immunotherapy of cancer with tumor-infiltrating lymphocytes. Science 233, 1318-1321 (1986).
7. Huang, J. et al. Survival, persistence, and progressive differentiation of adoptively transferred tumor-reactive T cells associated with tumor regression. J Immunother 28, 258-267 (2005).
8. Powell, D. J., Dudley, M. E., Robbins, P. F. & Rosenberg, S. A. Transition of late-stage effector T cells to CD27+ CD28+ tumor-reactive effector memory T cells in humans after adoptive cell transfer therapy. Blood 105, 241-250 (2005).
9. Dudley, M. E., Wunderlich, J. R., Shelton, T. E., Even, J. & Rosenberg, S. A. Generation of tumor-infiltrating lymphocyte cultures for use in adoptive transfer therapy for melanoma patients. J Immunother 26, 332-342 (2003).
10. Wu, R. et al. Adoptive T-cell therapy using autologous tumor-infiltrating lymphocytes for metastatic melanoma: current status and future outlook. Cancer J 18, 160-175 (2012).
11. Zhou, J., Dudley, M. E., Rosenberg, S. A. & Robbins, P. F. Persistence of multiple tumor-specific T-cell clones is associated with complete tumor regression in a melanoma patient receiving adoptive cell transfer therapy. J Immunother 28, 53-62 (2005).
12. Tran, K. Q. et al. Minimally cultured tumor-infiltrating lymphocytes display optimal characteristics for adoptive cell therapy. J Immunother 31, 742-751 (2008).
13. Pilling, D. et al. High cell density provides potent survival signals for resting T-cells. Cell Mol Biol (Noisy-le-grand) 46, 163-174 (2000).
14. Fischer, K. et al. Inhibitory effect of tumor cell-derived lactic acid on human T cells. Blood 109, 3812-3819 (2007).
15. Ghaffari, S. et al. Optimizing interleukin-2 concentration, seeding density and bead-to-cell ratio of T-cell expansion for adoptive immunotherapy. BMC Immunol 22, 43 (2021).

### Example 2: Comparison of TILs Produced on a Small Scale from Either the Traditional REP or a Feeder Cell-free TransAct REP Protocol

Traditionally, TILs were grown from single tumor fragments in wells of 24-well plates with a high dose of IL-2, selected for tumor-reactivity and then rapidly expanded⁶. Even though products from this method are clinically effective, TIL production is challenging because the traditional procedure of TIL production is assayed for specific tumor recognition and usually takes 6-8 weeks, which cause T exhaustion in vitro and short persistence in vivo^{7, 8}. The selection of tumor response reactivity also results in lower successful rate of producing TILs, leading to a more than 50% dropout rate of patients referred for TIL therapy⁹, largely limiting its clinical application¹⁰. An alternative “young” TIL process has been developed using whole tumor tissue in tissue culture flasks and gas permeable bags that favors the generation of TILs with properties associated with improved in vivo persistence, such as long telomeres and increased expression of CD27 and CD28^{11, 12}.

The REP protocol used here was a feeder cell-free process that is different from the traditional TIL REP protocol in which irradiated PBMCs and anti-CD3 (OKT3) antibody are used for expansion. The feeder cell-free REP process can enhance the potency and safety of the product and reduce the manufacturing costs. We have performed comparisons of a small-scale TILs using the traditional REP protocol and from the feeder-free TransAct REP process.

### TIL Expansion in TransAct REP Vs Traditional REP

Using pre-REP TILs from 12 individual donors, we found that the average fold expansion of the TILs expanded using TransAct REP protocol, was 389.3±138.8 (Table 9), which is comparable with the Traditional REP protocol. This suggested that the TransAct REP protocol can generate sufficient TILs to meet clinical target dose requirements.

TABLE 9
TIL Fold-Expansion Using Traditional REP or TransAct REP

| Donor ID | REP scale* | Traditional REP | TransAct REP |
|---|---|---|---|
| T5101008 | mini | 344 | 356 |
| T5101014 | mini | 438 | 482 |
| T5101015 | mini | 525 | 437 |
| T5101016 | mini | 572 | 351 |
| T5101017 | mini | 707 | 423 |
| T5101018 | mini | 676 | 338 |
| T5101001 | midi | 420 | 528 |
| T5101002 | midi | 418 | 300 |
| T5101016 | midi | 530 | 172 |
| T5101021 | midi | 423 | 162 |
| T5101022 | midi | 598 | 491 |
| T5101024 | midi | 534 | 631 |

| | | |
|---|---|---|
| Mean ± SD | 515.4 ± 111.1 | 389.3 ± 138.8 |
| 95% CI | (444.9; 586.0) | (301.0; 477.5) |

*Pre-REP TIL cell starting number on Day 0: mini = 0.1 × 10⁶; midi = 0.5 × 10⁶; SD, standard deviation.

### Viability and T Cell Subtype of TransAct REP Vs Traditional REP

TransAct REP TILs showed a marked increase in the proportion of CD8⁺ T cells that was consistent among all donors (FIG. 5 and Table 10).

Comparison of T Cell Phenotype and Inhibitory/Activation Cell Surface Markers from TransAct REP or Traditional REP

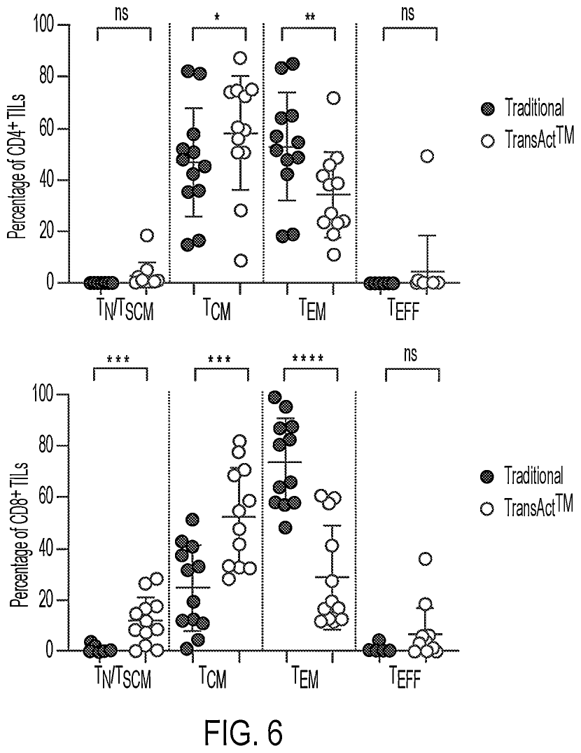

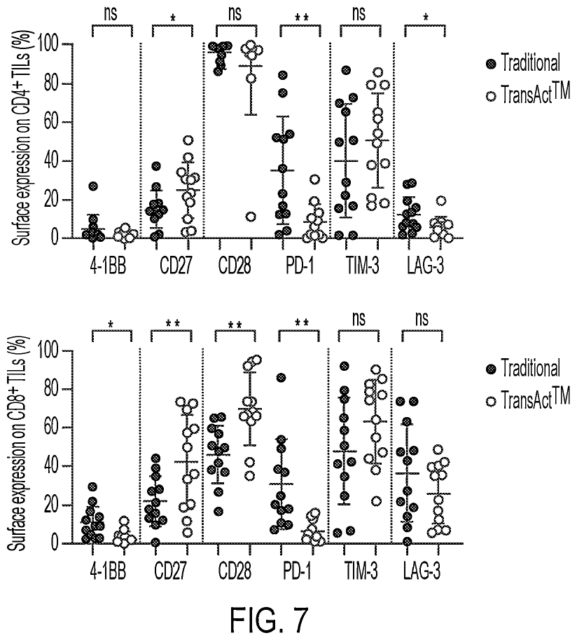

For TransAct REP TILs, both CD4⁺ and CD8⁺ populations showed significant increases in the percentage of cells with an early memory phenotype (FIG. 6, FIG. 7, and Table 10), while that of more differentiated effector T cells (T_{EM}, T_{EFF}) were decreased (FIG. 6 and Table 10). These TIL characteristics have been shown to be associated with favorable outcomes in TIL adoptive cell therapy⁴.

REP TILs were also analyzed for the expression of immune checkpoint (inhibitory) and activation cell surface markers (FIG. 7 and Table 10). Compared to traditional REP TILs, TransAct REP TILs had increased percentages of CD27⁺ cells in both CD4⁺ and CD8⁺ compartments and decreased percentages of exhaustion marker PD-1 expressing CD4⁺ and CD8⁺ T cells, which further supported the early memory phenotype of TransAct REP TILs.

TABLE 10
Summary of Percentages of T Cell Subtype and Phenotype
Markers of TILs Expanded by Traditional or TransAct REP

| | | Traditional* (%) | TransAct* (%) | |
|---|---|---|---|---|
| FACS Panel | T Cell Subtype | n = 12 | n = 12 | P values^{#} |
| T lineage | CD45⁺/CD3⁺ | 96.5 ± 2.1 | 97.6 ± 2.5 | 0.2251 |
| | CD3⁺/CD4⁺ | 92.8 ± 7.2 | 60.2 ± 30.0 | 0.0011 |
| | CD3⁺/CD8⁺ | 6.0 ± 5.8 | 36.7 ± 28.2 | 0.0013 |
| T memory | CD4⁺/T_{N/SCM} | 0.1 ± 0.1 | 3.0 ± 5.2 | 0.0752 |
| | CD4⁺/T_{CM} | 46.9 ± 20.9 | 58.2 ± 22.0 | 0.0184 |
| | CD4⁺/T_{EM} | 53.0 ± 20.9 | 34.3 ± 16.4 | 0.0058 |
| | CD4⁺/T_{EFF} | 0 ± 0 | 4.5 ± 14.1 | 0.2920 |
| | CD8⁺/T_{N/SCM} | 0.9 ± 1.3 | 11.9 ± 9.2 | 0.0009 |
| | CD8⁺/T_{CM} | 24.8 ± 16.7 | 52.3 ± 19.1 | 0.0002 |
| | CD8⁺/T_{EM} | 73.7 ± 16.9 | 29.0 ± 20.1 | <0.0001 |
| | CD8⁺/T_{EFF} | 0.7 ± 1.2 | 6.8 ± 10.4 | 0.0713 |
| T activation | CD4⁺/4-1BB⁺ | 5.0 ± 7.5 | 2.3 ± 1.5 | 0.1735 |
| | CD4⁺/CD27⁺ | 15.5 ± 9.7 | 25.1 ± 14.3 | 0.0166 |
| | CD4⁺/CD28⁺ | 96.1 ± 4.4 | 88.7 ± 24.7 | 0.2776 |
| | CD8⁺/4-1BB⁺ | 11.1 ± 8.2 | 3.4 ± 3.3 | 0.0220 |
| | CD8⁺/CD27⁺ | 22.0 ± 13.1 | 42.4 ± 24.4 | 0.0015 |
| | CD8⁺/CD28⁺ | 46.3 ± 15.0 | 69.8 ± 18.7 | 0.0024 |
| T inhibitory | CD4⁺/PD-1⁺ | 35.3 ± 27.6 | 9.0 ± 9.1 | 0.0029 |
| | CD4⁺/TIM-3⁺ | 40.2 ± 29.4 | 50.7 ± 24.4 | 0.1997 |
| | CD4⁺/LAG-3⁺ | 12.7 ± 8.9 | 6.1 ± 5.2 | 0.0315 |
| | CD8⁺/PD-1⁺ | 31.0 ± 23.2 | 6.4 ± 5.0 | 0.0022 |
| | CD8⁺/TIM-3⁺ | 48.0 ± 27.5 | 63.3 ± 21.6 | 0.1004 |
| | CD8⁺/LAG-3⁺ | 36.7 ± 25.0 | 26.1 ± 15.8 | 0.2413 |

*Values are presented as mean percentage ± standard deviation.
^{#}Statistical significance was determined by paired t test, n = 12. CD45⁺/CD3⁺, the percentage of CD3⁺ cells among CD45⁺ cells; CD4+ or CD8⁺/T_{N/SCM}, the sum percentage of T_{N} and T_{SCM} subsets among CD4⁺ or CD8⁺ cells; other subsets and inhibitory/activation markers are also given as percentage within CD4⁺ or CD8⁺; T_{N}, naive T cells; T_{SCM}, stem cell memory; T_{CM}, central memory; T_{EM}, effector memory; T_{EFF}, effector T cells.

Table 11 shows the T cell subsets from another experiment.

TABLE 11

| | irPBMC | Present Method | |
|---|---|---|---|
| T Cell Subtype | (avg %, range) | (avg %, range) | p-values |
| live/CD3⁺ | 90 (85.6-92.4) | 96.1 (92.8-98.5) | 0.000748 |
| CD3⁺/CD4⁺ | 91.4 (72.6-99) | 58.1 (10.3-95.7) | 0.003454 |
| CD3⁺/CD8⁺ | 7 (0.36-22.4) | 36.9 (3.27-88.4) | 0.005022 |
| CD4/T_{N} | 0.1 (0.013-0.22) | 1.6 (0.26-5.44) | 0.015843 |
| CD4/T_{N}/T_{SCM} | 0.1 (0.013-0.22) | 1.6 (0.26-5.44) | 0.015883 |
| CD4/T_{CM} | 55 (35.3-82.1) | 67.3 (50.7-87.4) | 0.038322 |
| CD4/T_{EM} | 44.9 (17.8-64.6) | 30.6 (11.2-48.9) | 0.027267 |
| CD4/T_{EFF} | 0 (0.00667-0.063) | 0.5 (0.13-1.27) | 0.003641 |
| CD8/T_{N} | 0.9 (0-3.93) | 12.2 (0.79-28.1) | 0.004956 |
| CD8/T_{N}/T_{SCM} | 0.9 (0-3.83) | 12.1 (0.78-27.8) | 0.004947 |
| CD8/T_{CM} | 27.2 (4.51-51.2) | 54.3 (28.6-82.3) | 0.002427 |
| CD8/T_{EM} | 71.1 (48.4-95.5) | 28.7 (11.8-60.7) | 2.17E−05 |
| CD8/T_{EFF} | 0.8 (0-4.34) | 4.8 (0.4-18.2) | 0.05756 |
| CD4/4-1BB⁺ | 5.4 (0.6-27.2) | 1.9 (0.23-5.71) | 0.195645 |
| CD4/CD27⁺ | 18.6 (10.5-37.2) | 29 (10.3-50.5) | 0.043882 |
| CD4/CD28⁺ | 96.3 (86.5-99.3) | 95.3 (82.8-99.7) | 0.349885 |
| CD8/4-1BB⁺ | 10.3 (2.67-21.8) | 3.8 (0.26-11.6) | 0.059625 |
| CD8/CD27⁺ | 24.2 (9.64-44.4) | 43.9 (12.4-72.3) | 0.008195 |
| CD8/CD28⁺ | 43.1 (16.8-64.9) | 70.5 (34.6-95.2) | 0.001735 |
| CD4⁺LAG3⁺ | 12 (2.16-28.2) | 6.3 (0.24-19.4) | 0.129779 |
| CD4⁺PD1⁺ | 44.4 (12.9-81.5) | 10.8 (0.99-30.4) | 0.001788 |
| CD4⁺TIM3⁺ | 43 (0.077-85.7) | 56.7 (17.8-85.5) | 0.228513 |
| CD8⁺LAG3⁺ | 40.4 (15.1-74.2) | 30.5 (6.03-51.2) | 0.152748 |
| CD8⁺PD1⁺ | 31.7 (7.9-60.2) | 8.3 (1.67-17.8) | 0.005761 |
| CD8⁺TIM3⁺ | 46.7 (1.48-93) | 72.8 (44.5-91.6) | 0.028425 |

Percentages of REP TIL T cell subtypes as determined by FACS analysis from nine donors expanded with irradiated PBMC (irPBMC) + OKT3 mAb or the present method (e.g., using MACS ®GMP T Cell TransAct ™). Shown are the average percentages of each population with the range in parentheses. P-values were determined using a paired t-test in Excel (n = 9).

### T Cell Activation in Response to αCD3 Stimulation of TransAct REP Vs Traditional REP

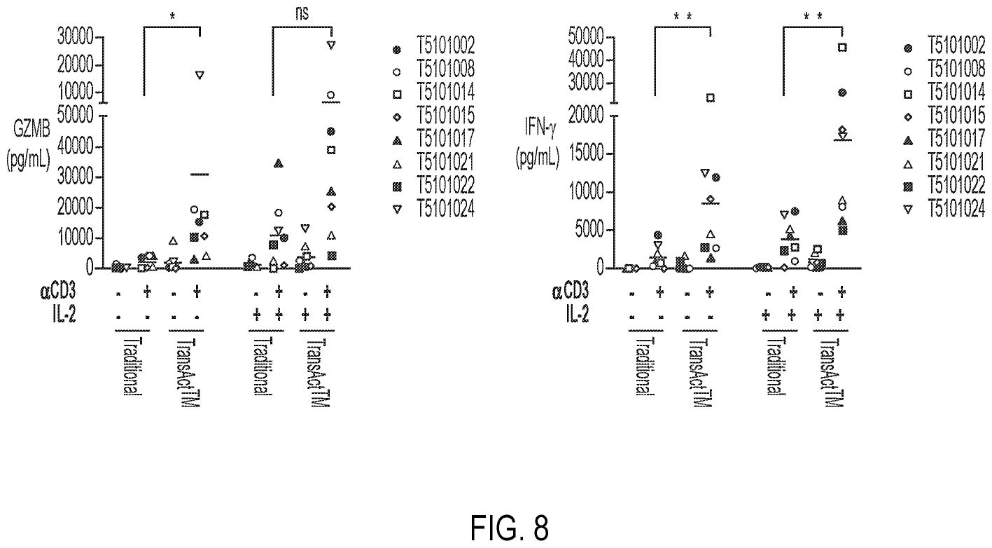

To compare the functionality of TILs expanded with the two REP methods, we examined the change of cell surface activation markers and cytokine secretion of the harvested cells from REP TILs in response to αCD3 stimulation. TransAct REP TILs produced much higher levels of IFN-γ and Granzyme B upon αCD3 stimulation with or without exogenous IL-2 (FIG. 8, Table 12). In the presence or absence of exogenous IL-2, the average proportion of TILs expressing surface 4-1BB was greater in TransAct REP TILs, in both CD4⁺ and CD8⁺ populations (Table 12). These data demonstrate that compared to traditional REP, TransAct REP supports better functionality of expanded TILs in response to T cell activation.

TABLE 12
Summary of Percentage of 4-1BB⁺ among CD4⁺ or CD8⁺ TILs after
Activation with anti-CD3 (OKT3) Antibody in the Presence or Absence of Exogenous IL-2

| | no IL-2 | | +IL-2 | | |
|---|---|---|---|---|---|
| REP method | no OKT3 | +OKT3 | no OKT3 | +OKT3 | |
| % 4-1BB⁺ | Traditional | 1.63 ± 1.18 | 9.64 ± 3.85 | 6.01 ± 7.29 | 25.45 ± 10.37 |
| among CD4⁺ | TransAct | 1.69 ± 1.45 | 26.85 ± 13.03 | 3.63 ± 2.39 | 36.13 ± 14.71 |
| TILs | P value* | 0.934 | 0.007 | 0.385 | 0.102 |
| % 4-1BB⁺ | Traditional | 3.59 ± 1.94 | 56.45 ± 11.47 | 9.51 ± 6.45 | 64.61 ± 16.32 |
| among CD8⁺ | TransAct | 3.6 ± 4.64 | 63.21 ± 13.55 | 6.5 ± 6.56 | 74.69 ± 11.07 |
| TILs | P value* | 0.994 | 0.396 | 0.488 | 0.248 |

*Data are presented as mean ± standard deviation; P values were determined by Student's paired t test; n = 8.

In Vitro MOS Tumor-Killing of TILs from TransAct REP or Traditional REP

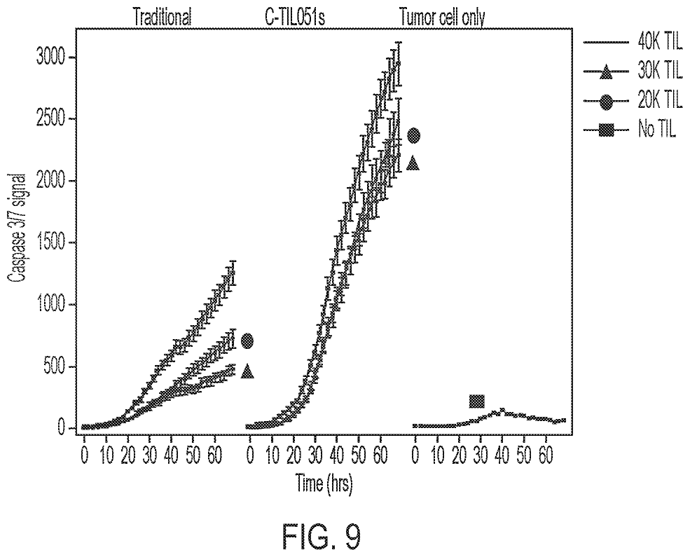

Co-culturing of TILs with autologous tumor-derived micro-organospheres (MOS) was performed to compare the tumor cell killing efficacy of traditional and TransAct REP TILs. REP TILs from Donor T5101016 were co-cultured with autologous lung cancer MOS with different E:T ratios of 2:1, 3:1 or 4:1 (20,000-40,000 TILs: 10,000 cancer cells in MOS). The co-culture was imaged every 2 hours for up to 68 hours and increasing Caspase 3/7 signals were observed in TIL groups (FIG. 9).

The assay confirmed the cytotoxicity of TILs against autologous NSCLC MOS. TransAct REP TILs (C-TIL051) showed significantly increased tumor killing activity, as indicated by Caspase 3/7 signal, in all E:T ratios tested in comparison with traditional TILs (FIG. 9). These data demonstrate that compared to traditional REP TILs, TransAct REP TILs have better functionality in response to autologous tumor cells.

### TCR Repertoire of TransAct REP TILs Vs Traditional REP TILs

TCR beta chain sequencing data of TILs from 6 donors were compared for repertoire diversity before (pre-REP) and after REP expansion using either traditional or TransAct REP. Morisita Index (which is a correlation analysis considering both the number and relative abundance of shared clones between two samples) is presented for each pre-REP and REP sample pair. The value ranges from 0 to 1, and high values indicate high similarity between the overall repertoire of the two samples. The Morisita Indices of TransAct REP were higher than that of traditional REP, suggesting that TransAct REP may overall preserve the pre-REP repertoire to a higher extent (Table 13).

TABLE 13
Morisita Indices of REP TILs vs pre-REP TILs from
the Analysis of TCR Beta Chain Sequencing Data

Morisita Indices

| | Small Scale | Small Scale |
|---|---|---|
| | Traditional | TransAct |
| Sample ID | REP | REP |
| T5101015 | 0.07 | 0.24 |
| T5101016 | 0.21 | 0.19 |
| T5101018 | 0.49 | 0.12 |
| T5101021 | 0.31 | 0.54 |
| T5101022 | 0.64 | 0.86 |
| T5101024 | 0.19 | 0.53 |
| Mean | 0.32 | 0.41 |
| *STDEV | 0.21 | 0.28 |

*STDEV, standard deviation.

In addition, the composite analyses with different matrices from multiple aspects (including Simpson clonality, TCR overlap between pre-REP TILs and TILs, preservation of top 100 pre-REP clones after REP) demonstrate that the TransAct REP process appeared to be better than, or at least not inferior to, the traditional REP process in preserving the pre-REP TCR repertoire, which further supports the use of TransAct in the REP process.

### Example 3: TIL REP Process Development and TIL Characterization

Our small-scale experiments using pre-REP TILs derived from the tumors of 11 NSCLC patients showed that MACS® GMP T Cell TransAct™, the commercially available αCD3/αCD28 agonist (abbreviated as TransAct herein), could be used in place of irradiated feeder cells and αCD3 antibody to support the rapid expansion of TILs, with the ability to meet clinical dose requirements. The purpose of this study was to develop a REP process using TransAct to expand pre-REP TILs at the manufacturing scale. We also aimed to characterize the TILs generated in process development (PD) runs and mock runs from the GMP facility for T cell phenotype, repertoire diversity, and/or tumor killing function.

Our experimental data show that, after activation with MACS® GMP T Cell TransAct™, successful REP of TILs can be achieved at manufacturing scale in either G-Rex® cell culture devices or with sequential use of G-Rex® and Xuri™ W25 bioreactor. Characterization of the TILs from process development runs showed that the expanded cells exhibited a favorable phenotype and a well-preserved T cell receptor (TCR) repertoire representative of cognate pre-REP TILs. The TILs also retained the ability to upregulate the expression of surface T-cell activation markers and produce various cytokines in response to T cell activation signals. Furthermore, characterization of the TIL products from mock runs from our GMP facility demonstrated that the autologous cell therapy products we developed bear an early differentiated phenotype and are endowed with strong tumor killing ability.

### Materials

The pre-REP TILs derived from 11 donors were used for PD. The pre-REP cells from 5 donors (T5101001, T5101002, T5101005, T5101008, T5101010) were harvested before pre-REP process optimization, of which CD3⁺ T cell percentage ranged from 38.7% to 98.0%; the pre-REP cells from the other 6 donors (T5101014, T5101015, T5101016, T5101017, T5101018, T5101024) were harvested after pre-REP process optimization, of which CD3⁺ T cell percentage ranged from 81.8% to 97.0%.

### Methods

REP with TransAct and Sequential Use of G-Rex®10M and G-Rex® 100M Devices

On Day 0, pre-REP cells were thawed in a water bath and washed twice with REP complete medium (CM, REP basal medium supplemented with 300, or 1000, 3000, or 6000 IU/mL IL-2). In one embodiment, the IL-2 concentration is 300 IU/mL. In another embodiment, the IL-2 concentration is 1000 IU/mL. In yet another embodiment, the IL-2 concentration is 3000 IU/mL. In still another embodiment, the IL-2 concentration is 6000 IU/mL. Cells were then resuspended with REP CM and counted on an automated cell counter (NC-200). To start the culture in a G-Rex®10M device, an aliquot of cell suspension containing about 1×10⁶, 2×10⁶, 5×10⁶, 10×10⁶ or 20×10⁶ live cells and TransAct were added to the device with a volume ratio of about 1:150, 1:5, 1:10 or 1:17.5 TransAct: medium, and the final culture volume was adjusted to 10 mL, 15 mL, or 20 mL with REP CM. The G-Rex®10M was then placed in an incubator at 37° C. and 5% CO₂.

On Day 3, 4, or 5, the initial culture was diluted 10-fold, 20-fold, or 30-fold by adding pre-warmed REP CM to the G-Rex®10M. The device was returned to the CO₂ incubator.

On Day 6, 7, 8, 9 or 10, after resuspending and counting the cells in the G-Rex®10M, the culture was transferred to a new G-Rex® 100M device and diluted 10-fold, 20-fold, or 30-fold by adding pre-warmed REP CM. The G-Rex®100M was then placed in the CO₂ incubator.

On Day 12, 13, 14, 15, 16, 17, 18, 19 or 20, cells were harvested from the G-Rex®100M. Aliquots of remaining cells were pelleted and cryopreserved.

REP with TransAct and Sequential Use of a G-Rex® Device and Xuri™ W25

On Day 0, pre-REP cells were thawed in a water bath and washed twice with REP CM. Cells were then resuspended with REP CM and counted on an NC-200. To start the culture in a G-Rex®100M device, an aliquot of cell suspension containing about 20×10⁶, 50×10⁶, 1000×10⁶ or 200×10⁶ live cells and TransAct was added to the device with a volume ratio of about 1:150, 1:5, 1:10 or 1:17.5 TransAct: medium, and the final culture volumes were adjusted to 50-100 mL with REP CM. Alternatively, if using the closed system G-Rex®100M-CS device, the barrel of a 50 ml syringe was connected to the Luer port of the media addition tubing and then the cell suspension and TransAct were added to the G-Rex® through the syringe barrel. After that, the media addition tubing was heat sealed and the syringe barrel removed. The G-Rex®100M or G-Rex®100M-CS were then placed in a CO₂ incubator.

On Day 3, 4, or 5, the initial culture was diluted 10-fold, 15-fold, or 20-fold by adding pre-warmed REP CM to the G-Rex®100M. If using the G-Rex®100M-CS, media was added via a media transfer bag containing REP CM welded to the media addition tubing on the G-Rex®100M-CS. Following the addition of media, the tubing was heat sealed and the media transfer bag was removed. The G-Rex® device was returned to the CO₂ incubator.

On about Day 6-10, about 25%, or 50% of spent media was removed from the top of the G-Rex®100M and saved in a new media bottle. If using the G-Rex® 100M-CS, the spent media was removed via the media addition/removal tubing and saved in a media transfer bag. Cells were then resuspended in the G-Rex® device and a sample was taken for counting on an NC-200. The G-Rex® device was returned to the CO₂ incubator until harvest.

To start Xuri™ REP, a 2L or 10L Xuri™ Cellbag was installed on the rocking tray of the Xuri™ W25 Cell Expansion System. Fresh CM was then added to the Cellbag, and the system was allowed to equilibrate for 2 hours.

Depending on the total cell number, an aliquot of the cell suspension in the G-Rex® device was harvested into a transfer bag (for G-Rex®100M-CS, the GatheRex cell harvest pump was used) so that the cell number met the requirement of the Xuri™ Cellbag inoculation. The transfer bag was then connected to the Feed line of Xuri™ Cellbag to transfer the cells. Additional spent media from the G-Rex® (saved before cell counting) was added to the Xuri™ Cellbag so that the volume of fresh CM in the Cellbag would be one-half of the final total culture volume, and the final cell concentration would be about 0.5×10⁶/mL or 1×10⁶/mL or 2×10⁶/mL. After that, the Xuri™ expansion was started with rocking at a low speed and angle.

Day 6, 7 or 8 and thereafter: samples were taken every day from the Xuri™ Cellbag to count and monitor the cell expansion and viability. Fresh REP CM was added after each cell count to adjust the culture to the desired cell concentration until the total culture volume reached 1 or 5 liters. Perfusion was then started the next day at 0.5 liter/day, 1 liter/day or 2 liter/day. The perfusion rate along with the rocking speed and angle was gradually increased daily according to cell concentration.

On the last day of Xuri™ culture, after taking samples for cell count and FACS analysis, the REP cells were harvested, washed, and cryopreserved.

### αCD3 Stimulation

The REP products of 3 donors (T5101015, T5101018, and T5101024) were thawed and rested in REP CM for 1-2 days. Cells were then washed and resuspended in REP Basal Media either without IL-2 or with 300 IU/mL IL-2 (final concentration), and 2×10⁵ cells were seeded in one well of a 96-well plate. MACS® GMP CD3 pure (αCD3 antibody, OKT3) was diluted in the same culture media and added to the wells at a final concentration of 1 μg/mL. Cells were stimulated overnight, then supernatants were taken for cytokine assay, and cells were stained for FACS analysis. The experiment was run in triplicate for each condition.

### Cytokine Assay

For analysis of cytokine concentrations in cell culture supernatants, the LEGENDplex™ Human CD8/NK panel kit was used for simultaneous quantification of multiple soluble analytes following the manufacturer's protocol. The assay was read on the CytoFLEX LX FACS instrument, and data were analyzed with the LEGENDplex™ software.

### FACS

Cells were first stained with LIVE/DEAD™ Fixable Aqua dye, followed by staining with antibodies against various surface markers. Reagents. Samples were then acquired by the CytoFLEX LX FACS instrument. All data were analyzed with FlowJo V10 software.

### Collection of TIL Culture Medium Samples

For REP with G-Rex® devices where cells reside at the bottom, about 500 μL of the supernatant was sampled at the indicated time points. For REP with the Xuri™ W25 bioreactor, culture samples were collected from the sampling port of the Xuri™ Cellbag and centrifuged at 400 g/min for 5 min. REP culture medium samples (200 μL-500 μL) were acquired by transferring the supernatant to a new 1.5-mL sterile tube. The culture medium samples were immediately stored in a −20° C. freezer and thawed on ice at the time of the assays.

### IL-2 Enzyme-Linked Imnnunosorbent Assay (ELISA)

Samples were diluted 15˜300 times with REP basal medium depending on IL-2 protein concentration. Samples were measured in duplicate or triplicate wells, and data are presented as Mean±Standard Deviation (S.D.). The mean value of IL-2 concentration in three batches of fresh REP CM samples was designated as Day 0 concentration. The mass concentrations were converted to unit concentrations provided that 1 ng/mL of IL-2 is equivalent to 16.36 IU/mL.

### Quantitation of Glucose and Lactate Concentration in Culture Supernatants

Samples were aliquoted and analyzed with Glucose-Glo and Lactate-Glo Assay kits following the manufacturer's recommended protocol. Culture supernatants were diluted in PBS at 1:200 to 1:300. Plates were read for luminescence in a SpectraMax iD3. Standard curves were generated in GraphPad Prism and values of samples were calculated in Excel.

### TCR Repertoire Analysis

TCR beta chain sequencing of TILs from 2 donors was performed to compare the TCR repertoire diversity before (pre-REP) and after full scale REP expansion of PD runs with sequential use of G-Rex® device and Xuri™ W25 bioreactor (T5101015-3 and T5101024a).

Mock Run of TILs from GMP Facility

Pre-REP TILs from 2 donors (T5101034 and T5101035) were expanded and cryopreserved in a GMP facility. CD3⁺ T cell percentages of the pre-REP TILs were 85.72% and 90.82% for T5101034 and T5101035, respectively. The pre-REP TILs were cryopreserved for 10 days and 25 days, respectively, before starting REP production in the same GMP facility. The standard operating procedures of REP were developed based on the PD work and applied to the engineering runs and mock runs.

### Characterization of TIL Mock Run Products

The yield of TIL mock run production was determined by automated cell counting on an NC-200 cell counter. T cell phenotype of the products was analyzed by surface marker staining and FACS on a BD FACSLyric™ Clinical Flow Cytometry System. To determine the non-specific activation, the cryopreserved products were thawed and recovered overnight, then 1×10⁵ cells were stimulated with Dynabeads™ Human T-Activator CD3/CD28 (Thermo Fisher) at a 1:1 ratio in a final volume of 200 μL for 24 hrs. Cell supernatant was then harvested for IFN-γ ELISA using a commercial kit (Thermo Fisher). Alternatively, after overnight recovery, 2×10⁵ cells were stimulated with αCD3 antibody (Miltenyi) at a final concentration of 1 μg/mL in a final volume of 200 μL for 24 hrs. Cells were then harvested for FACS analysis of T cell activation markers including 4-1BB and OX40.

The TIL mock run products of 2 donors (T5101034 and T5101035) were also characterized by a direct functional assay involving co-culturing of the TIL products with autologous tumor-derived micro-organospheres (MOS) (Ding et al. Patient-derived micro-organospheres enable clinical precision oncology, Cell Stem Cell, 2022, 29, 905-917, e906).

### Results and Analysis

C-TIL051 PD Run with TransAct and Sequential Use of G-Rex®10M and G-Rex®100M Devices

We first preformed REP using 10×10⁶ pre-REP TILs from 10 donors with TransAct and sequential use of G-Rex®10M and G-Rex®100M devices.

### Conditions of the REP Process

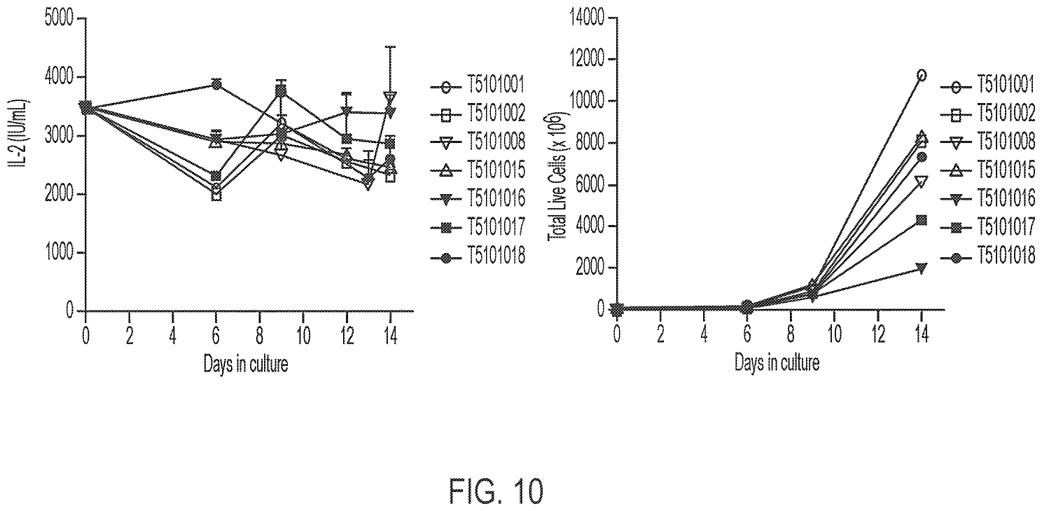

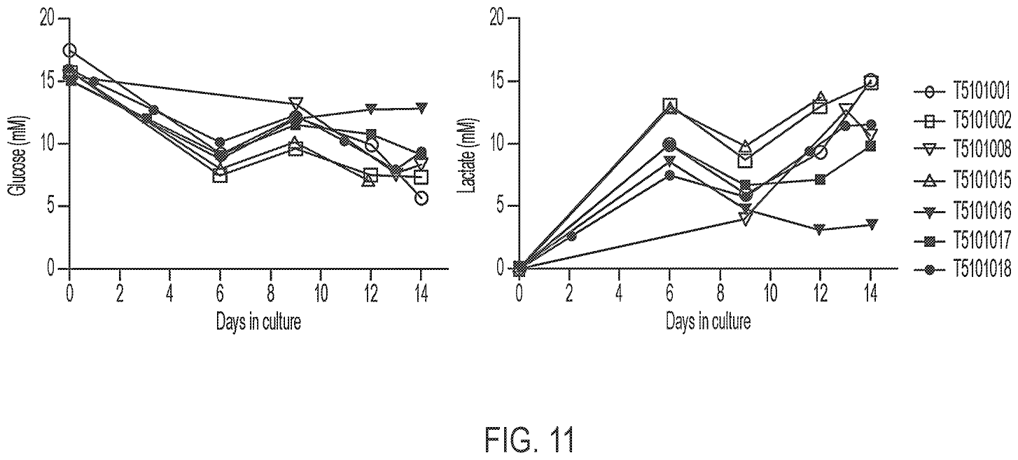

The REP of TILs requires sufficient nutrients and high IL-2 concentration, whereas the accumulation of metabolites such as lactate may dampen T cell proliferation. We measured the levels of glucose, lactate and IL-2 from culture supernatant sampled before media change/addition and at harvest. The IL-2 concentration was found to be at high levels (>2000 IU/mL) at all time points for all samples tested, irrespective of cell proliferation rate (FIG. 10, left). Glucose levels decreased during the first 6 days of culture as TILs started to expand; after that media was added or exchanged, which maintained glucose levels to around 10 mM for most samples until the end of culture (FIG. 11), and we observed exponential expansion starting from Day 6 (FIG. 10, right). Consistent with the consumption of glucose, lactate started to accumulate in the culture during the first 6 days, whereafter it was diluted or maintained at similar, low levels due to the media addition/exchange. Overall, the lactate levels remained well below 20 mM for all samples tested throughout the culturing process (FIG. 11), which can be well tolerated by T cells according to a previous study¹⁴. These data demonstrated that the feeding schedule we developed meets the needs of TIL expansion in the G-Rex® devices.

The Fold Expansion, Viability, and T Cell Composition of TILs from PD Runs with TransAct and Sequential Use of G-Rex®10M and G-Rex®100M Devices

After the culture, TILs from 8 donors were expanded by at least 200-fold (Table 14), demonstrating successful REP. For these samples, we achieved an average fold expansion of 712.4±292.2 (95% confidence interval (CI): 468.1 to 956.7), as well as high cell viability (97.95%±1.51%) and T cell purity as determined by the CD3⁺ percentage among total live cells (97.43%±1.50%; Table 14). The pre-REP yield of these samples showed a broad range (30×10⁶˜783×10⁶ TILs), therefore the yield of pre-REP TILs does not appear to be associated with REP yield, and 2 samples with a pre-REP yield of less than 50 million cells (T5101002 and T5101008) displayed robust expansion with our protocol (Table 14). Given the high average fold expansion, the REP process developed here is expected to amplify as low as 10×10⁶ intermediate product of pre-REP TILs to achieve the target dose of final REP product, i.e., 1×10⁹ to 100×10⁹ cells. Notably, these 8 samples had greater than 80% CD3⁺ T cells in the starting material of pre-REP TILs (Table 14). By contrast, the other 2 pre-REP samples with low percentage of CD3⁺ T cell (T5101005 and T5101010, CD3⁺ T cells %: 64.0% and 38.7%, respectively) showed poor expansion at REP stage (17-fold and 167-fold, respectively). Thus, the percentage of CD3⁺ T cells among pre-REP TILs may be important for TransAct-mediated REP. Overall, these data demonstrated that TransAct can indeed support the REP of NSCLC TILs using G-Rex® devices at manufacturing scale with a high success rate. The successful expansion of the 8 samples also demonstrated that the REP process with TransAct can be started immediately after thawing of pre-REP cells, without recovery for 2 or more days, which is generally required for traditional REP with irradiated feeder cells.

TABLE 14
TIL Fold Expansion, Viability, and T Cell Composition from
PD Runs Using TransAct and G-Rex ® Devices

| | Pre-REP TILs | | REP TILs from PD runs | | | |
|---|---|---|---|---|---|---|
| Sample | Yield | CD3⁺ | Viability | CD3⁺ | CD3⁺ CD8⁺ | |
| ID | (×10⁶) | (%) | (%) | (%) | (%) | Fold* |
| T5101001 | 178.0 | 98.0 | 97.8 | 97.5 | 21.5 | 1125 |
| T5101002 | 34.1 | 97.4 | 99.5 | 98.1 | 10.4 | 804 |
| T5101008 | 30.0 | 89.7 | 98.4 | 96.5 | 42.6 | 612 |
| T5101014 | 783.0 | 83.0 | 99.2 | 98.9 | 46.9 | 950 |
| T5101015 | 459.0 | 85.9 | 95.8 | 96.1 | 40.9 | 839 |
| T5101016 | 148.0 | 91.2 | 95.5 | 98.6 | 80.8 | 205 |
| T5101017 | 275.0 | 81.8 | 98.7 | 94.8 | 38.0 | 432 |
| T5101018 | 216.0 | 92.0 | 98.7 | 98.9 | 96.4 | 732 |

| | | | | |
|---|---|---|---|---|
| Mean ± SD | 97.95 ± 1.51 | 97.43 ± 1.50 | 47.19 ± 28.56 | 712.4 ± 292.2 |
| 95% confidence interval | (96.69; 99.21) | (96.17; 98.68) | (23.31; 71.06) | (468.1; 956.7) |

*Cell fold expansion relative to total starting cell number; CD3⁺ (%), percentage of CD3⁺ cells among CD45⁺ live cells; CD3⁺ CD8⁺ (%), percentage of CD8⁺ cells among CD3⁺ cells.

T Cell Phenotype of TILs from PD Runs with TransAct and Sequential Use of G-Rex®10M and G-Rex®100M Devices

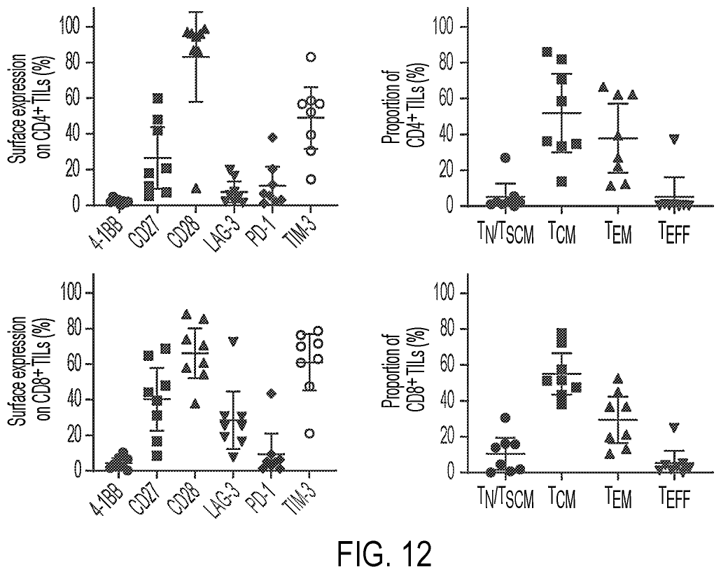

FACS analysis showed that the REP TILs from the PD runs with sequential use of G-Rex®10M and G-Rex®100M devices maintained a relatively high percentage of CD8⁺ cells (average 47.19% 28.56%; Table 14). Both CD4⁺ and CD8⁺ TILs expressed high levels of CD28, the predominant co-stimulatory molecule (average 83.10%±30.07% and 66.03%±16.81%, respectively; FIG. 12 and Table 15), and to a lesser extent, CD27 (average 26.46% 20.68% and 40.12%±21.15%, respectively; FIG. 12 and Table 15). In terms of inhibitory markers, both populations displayed low expression of PD-1 (average 10.98%±12.60% and 9.26%±14.03%, respectively; FIG. 12 and Table 15) and LAG-3 (average 7.46%±6.99% and 28.41%±19.40%, respectively; FIG. 12 and Table 15), but high expression of TIM-3 (average 48.96%±20.70% and 61.04%±18.97%, respectively; FIG. 12 and Table 15). Finally, the REP TILs were largely composed of central memory (T_{CM}) and effector memory (T_{EM}) cells, characteristic of cells with preserved proliferative capacity (FIG. 12). The average proportions of T_{CM} among CD4⁺ and CD8⁺ TILs were 51.88%±26.19% and 54.96%±13.82%, respectively; and that of T_{EM} were 37.81%±23.11% and 29.31%±15.44%, respectively (Table 16).

TABLE 15
Surface Expression of Select Activation and Inhibitory Markers
on CD4⁺ and CD8⁺ TILs after REP with TransAct Using
G-Rex ® Devices

| | | 4- | | | LAG- | PD- | TIM- |
|---|---|---|---|---|---|---|---|
| | Sample ID | 1BB | CD27 | CD28 | 3 | 1 | 3 |
| Expression | T5101001 | 4.7 | 5.2 | 86.8 | 16.6 | 1.1 | 39.5 |
| on CD4⁺ | T5101002 | 1.9 | 10.7 | 9.6 | 1.4 | 2.1 | 83.0 |
| TILs (%) | T5101008 | 1.9 | 59.9 | 98.6 | 5.6 | 20.4 | 56.8 |
| | T5101014 | 2.0 | 48.0 | 96.0 | 2.1 | 5.2 | 52.1 |
| | T5101015 | 3.1 | 18.0 | 86.2 | 7.4 | 11.5 | 56.7 |
| | T5101016 | 2.3 | 20.7 | 96.2 | 4.7 | 6.5 | 14.5 |
| | T5101017 | 1.6 | 41.9 | 94.4 | 19.8 | 38.0 | 58.6 |
| | T5101018 | 0.2 | 7.3 | 97.0 | 2.1 | 3.1 | 30.5 |
| | Mean | 2.22 | 26.46 | 83.10 | 7.46 | 10.98 | 48.96 |
| | SD | 1.30 | 20.68 | 30.07 | 6.99 | 12.60 | 20.70 |
| Expression | T5101001 | 10.3 | 8.5 | 54.2 | 30.7 | 1.3 | 60.6 |
| on CD8⁺ | T5101002 | 3.7 | 31.2 | 37.7 | 25.3 | 4.9 | 69.9 |
| TILs (%) | T5101008 | 1.5 | 64.7 | 60.6 | 25.2 | 9.3 | 62.9 |
| | T5101014 | 7.2 | 68.6 | 70.5 | 30.4 | 6.4 | 78.6 |
| | T5101015 | 2.0 | 16.5 | 73.9 | 19.1 | 2.8 | 47.5 |
| | T5101016 | 2.2 | 44.1 | 85.4 | 16.4 | 5.1 | 21.0 |
| | T5101017 | 6.6 | 39.5 | 57.9 | 72.5 | 43.3 | 71.5 |
| | T5101018 | 0.2 | 47.9 | 88.0 | 7.7 | 1.0 | 76.3 |
| | Mean | 4.22 | 40.12 | 66.03 | 28.41 | 9.26 | 61.04 |
| | SD | 3.46 | 21.15 | 16.81 | 19.40 | 14.03 | 18.97 |

TABLE 16
Memory Phenotype of CD4⁺ and CD8⁺ TILs after REP
with TransAct Using G-Rex ® Devices

| | Sample ID | T_{N}/T_{SCM} | T_{CM} | T_{EM} | T_{EFF} |
|---|---|---|---|---|---|
| Proportion | T5101001 | 0.0 | 33.4 | 66.5 | 0.1 |
| of CD4⁺ | T5101002 | 26.9 | 13.8 | 22.1 | 37.2 |
| TILs (%) | T5101008 | 5.4 | 81.9 | 12.2 | 0.6 |
| | T5101014 | 2.4 | 86.0 | 11.2 | 0.5 |
| | T5101015 | 1.1 | 58.6 | 39.2 | 1.1 |
| | T5101016 | 2.1 | 34.6 | 62.1 | 1.3 |
| | T5101017 | 2.2 | 70.6 | 27.0 | 0.3 |
| | T5101018 | 1.0 | 36.1 | 62.2 | 0.6 |
| | Mean | 5.14 | 51.88 | 37.81 | 5.20 |
| | SD | 8.93 | 26.19 | 23.11 | 12.94 |
| Proportion | T5101001 | 0.0 | 47.6 | 52.4 | 0.0 |
| of CD8⁺ | T5101002 | 15.8 | 38.1 | 21.1 | 25.0 |
| TILs (%) | T5101008 | 30.6 | 51.3 | 13.0 | 5.1 |
| | T5101014 | 13.9 | 72.6 | 10.4 | 3.2 |
| | T5101015 | 1.8 | 51.6 | 44.8 | 1.7 |
| | T5101016 | 16.1 | 43.2 | 36.7 | 4.1 |
| | T5101017 | 4.6 | 57.6 | 36.6 | 1.2 |
| | T5101018 | 0.7 | 77.7 | 19.5 | 2.1 |
| | Mean | 10.44 | 54.96 | 29.31 | 5.30 |
| | SD | 10.63 | 13.82 | 15.44 | 8.12 |

Taken together, the TransAct REP process we developed with sequential use of G-Rex®10M and G-Rex®100M devices can be applied for manufacturing of TIL cell therapy products, meeting the cell dose requirement while maintaining a favorable T cell phenotype.

C-TIL051 PD Runs with TransAct and Sequential Use of G-Rex® Devices and Xuri™ W25 Bioreactor

The TransAct REP process with sequential use of G-Rex®10M and G-Rex®100M devices process works well for donors with low pre-REP yield. We also developed a TransAct REP process with the sequential use of G-Rex®100M and Xuri™ W25 bioreactor for high-yield pre-REP TILs.

We performed 5 REP runs with pre-REP TILs from 3 donors. After seeding the pre-REP TILs in one G-Rex®100M or its closed system equivalent G-Rex®100M-CS (range of cell number seeded: 87×10⁶˜200×10⁶), cells were activated with TransAct and expanded in the G-Rex® device for 7 days, which resulted in an average of 12-fold cell expansion. The cultures were then transferred to a 10L Xuri™ Cellbag for further expansion with the Xuri™ W25 system except for T5101024, of which the cultures were equally distributed to two 10L Xuri™ Cellbags to compare different feeding schedules.

### Culture Conditions

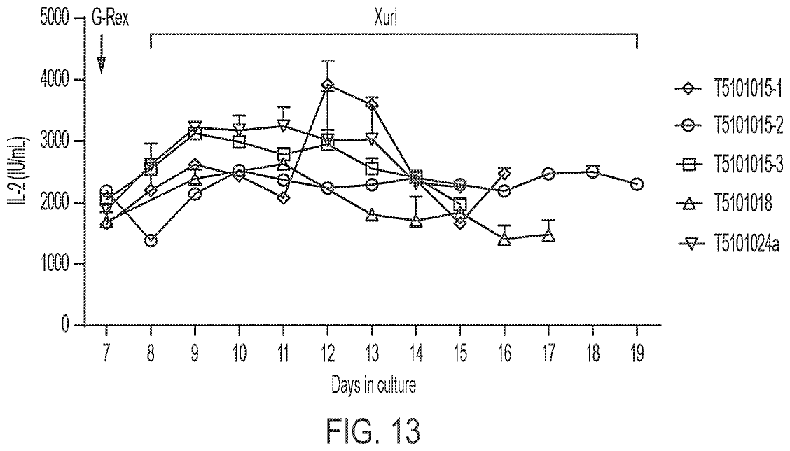

TIL expansion in G-Rex® devices and then in Xuri™ W25 bioreactor requires different feeding schedules. To justify the feeding schedule we developed, we measured the levels of glucose, lactate, and IL-2 from sampled supernatant of G-Rex® (Day 7) and Xuri™ (daily) cultures. The IL-2 concentration was found to be at high levels (>1000 IU/mL) at all time points for all samples tested, irrespective of cell proliferation rate (FIG. 13).

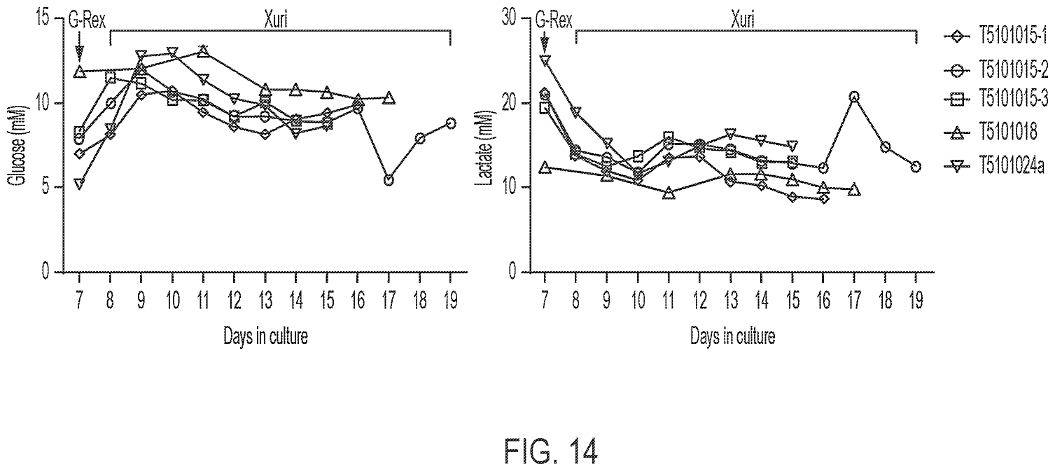

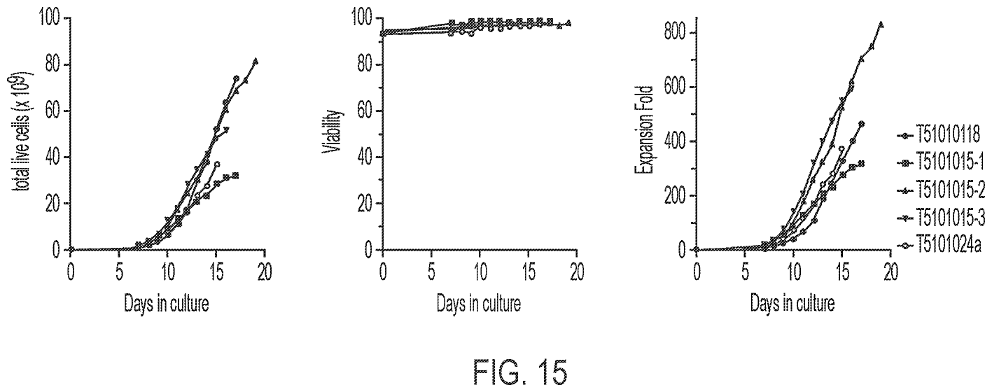

The glucose levels were also high (FIG. 14), while lactate was at low levels largely tolerated by T cells, except T5101024a REP which experienced a transitory, high level of lactate on Day 7 at the end of G-Rex® culture (FIG. 14). For this REP, the lactate level decreased and remained at low levels during Xuri™ culture (FIG. 14), as a result of addition of fresh REP CM, and we observed robust cell expansion (FIG. 15). These data demonstrated that the feeding schedule we developed meets the needs of TIL expansion in both the G-Rex® device and Xuri™ bioreactor.

TIL Yield, Viability, and T Cell Composition from PD Runs with TransAct and Sequential Use of G-Rex® Devices and Xuri™ W25 Bioreactor

After 8 to 12 days of Xuri™ expansion, the PD runs were terminated with a minimal REP yield of 32.4×10⁹ cells that reached the target dose range of 1×10⁹ to 100×10⁹ (average yield 55.38±21.91×10⁹; FIG. 15 and Table 17). The final REP product also maintained high viability (average 98.42%±0.08%) and CD3⁺ percentage (average 97.64%±0.95%), and the average cell expansion fold of this REP process is 514.6±206.2 (Table 17). Notably, these cells retain the potential to be further expanded as their proliferation plateau had not been reached when we terminated the process (FIG. 15), and the average cell expansion fold could be further increased by extending the culture time in Xuri™ bioreactor. Similarly, for samples that proliferate rapidly and reach target cell number early, the REP process can be stopped accordingly for harvest. Therefore, the REP process provides flexibility in future manufacturing time of TILs.

TABLE 17
TIL Yield, Viability, and T Cell Composition from REP PD Runs with TransAct
and Sequential Use of G-Rex ® Devices and Xuri ™ W25 Bioreactor.

| | Starting | C-TIL051 from PD runs | | | | |
|---|---|---|---|---|---|---|
| | cell | Viability | CD3⁺ | CD3⁺ CD8⁺ | Cell | |
| Sample ID | number | (%) | (%) | (%) | number | Fold* |
| T5101018 | 160 × 10⁶ | 98.3 | 99.0 | 94.4 | 74.3 × 10⁹ | 464 |
| T5101015-1 | 103 × 10⁶ | 98.4 | 97.6 | 33.0 | 32.4 × 10⁹ | 314 |
| T5101015-2 | 98 × 10⁶ | 98.5 | 97.1 | 39.6 | 81.5 × 10⁹ | 831 |
| T5101015-3 | 87 × 10⁶ | 98.4 | 96.5 | 36.0 | 51.7 × 10⁹ | 594 |
| T5101024a | 200 × 10⁶ | 98.5 | 98.0 | 55.5 | 37.0 × 10⁹ | 370 |

| | | | | | |
|---|---|---|---|---|---|
| Mean ± SD | 98.42 ± | 97.64 ± | 51.70 ± | (55.38 ± | 514.6 ± |
| | 0.08 | 0.95 | 25.40 | 21.91) × 10⁹ | 206.2 |
| 95% confidence interval | (98.32; | (96.47; | (20.16; | (28.18 × | (258.5; |
| | 98.52) | 98.81) | 83.24) | 10⁹; | 770.7) |

82.58 ×
10⁹)

*Cell expansion fold relative to total starting cell number on Day 0. For T5101024a, only ½ of the G-Rex ® culture were used to set up Xuri ™ run, and total expansion fold was obtained by multiplying the expansion fold of G-Rex ® and Xuri ™ stages. CD3⁺ (%), percentage of CD3⁺ cells among live CD45⁺ cells; CD3⁺ CD8⁺ (%); percentage of CD8⁺ cells among total CD3⁺ cells.

T Cell Phenotype of TIL PD Runs with TransAct and Sequential Use of G-Rex® Devices and Xuri™ W25 Bioreactor

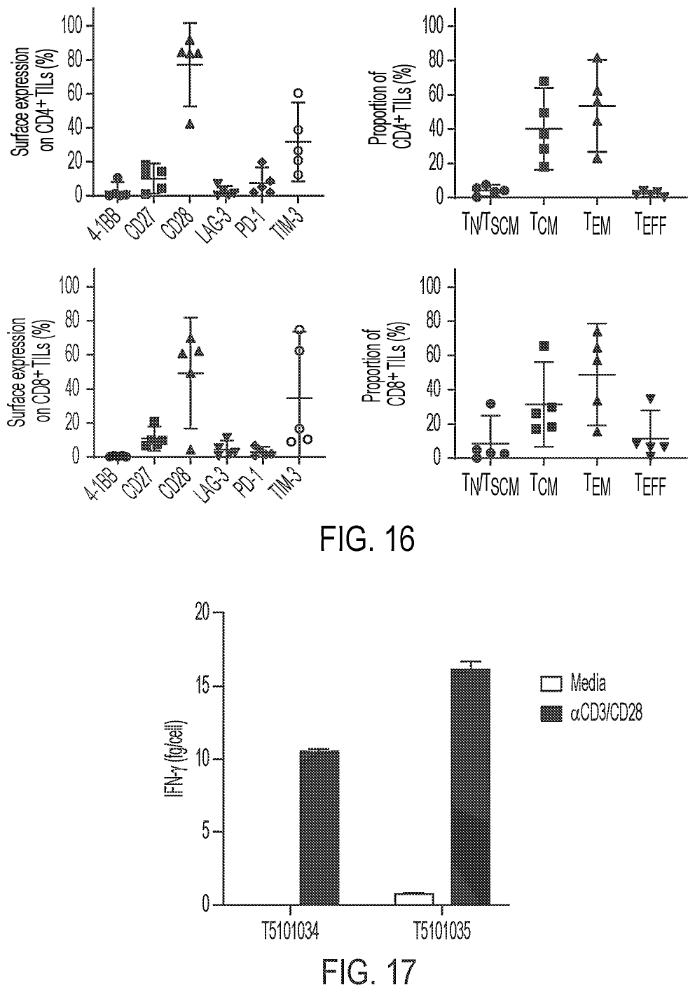

We analyzed the T cell phenotype by FACS, which revealed that the REP products from the new process also maintained a relatively high percentage of CD8⁺ cells (average 51.70% 25.40%; Table 17). Both CD4⁺ and CD8⁺ TILs expressed high levels of CD28 (average 77.14%±19.77% and 49.26%±26.18%, respectively), and medium levels of TIM-3 (average 31.72%±18.70% and 34.68%±31.40%, respectively), but low levels of 4-1BB, CD27, PD-1, and LAG-3 (average positivity around or below 10% for these markers; FIG. 16 and Table 18). The REP products were also largely composed of T_{CM} and T_{EM} cells (FIG. 16), bearing advantageous phenotype similar to TILs generated from the G-Rex® devices only process. The average proportions of T_{CM} among CD4⁺ and CD8⁺ TILs were 40.16%±19.32% and 31.34%±19.88%, respectively; and those of T_{EM} were 53.50%±21.66% and 48.86%±23.98%, respectively (Table 19).

TABLE 18
Surface Expression of Select Activation and Inhibitory Markers
on CD4⁺ and CD8⁺ TILs after REP with TransAct
and Sequential Use of G-Rex ® Device and Xuri ™ W25 Bioreactor

| | | 4- | | | LAG- | PD- | TIM- |
|---|---|---|---|---|---|---|---|
| | Sample ID | 1BB | CD27 | CD28 | 3 | 1 | 3 |
| Expression | T5101018 | 0.1 | 1.0 | 84.4 | 0.5 | 1.9 | 38.7 |
| on CD4⁺ | T5101015-1 | 1.2 | 12.9 | 83.6 | 7.0 | 19.7 | 20.8 |
| TILs (%) | T5101015-2 | 0.1 | 4.3 | 83.7 | 0.2 | 1.9 | 12.2 |
| | T5101015-3 | 10.5 | 14.2 | 91.7 | 2.6 | 8.7 | 26.5 |
| | T5101024a | 0.6 | 18.1 | 42.3 | 1.5 | 5.0 | 60.4 |
| | Mean | 2.50 | 10.11 | 77.14 | 2.34 | 7.45 | 31.72 |
| | SD | 4.50 | 7.16 | 19.77 | 2.76 | 7.39 | 18.70 |
| Expression | T5101018 | 0.2 | 20.9 | 49.2 | 1.9 | 0.9 | 62.4 |
| on CD8⁺ | T5101015-1 | 0.4 | 7.7 | 60.8 | 5.4 | 6.8 | 10.5 |
| TILs (%) | T5101015-2 | 0.3 | 6.6 | 62.2 | 0.8 | 1.6 | 9.1 |
| | T5101015-3 | 0.9 | 10.0 | 69.8 | 2.8 | 4.0 | 16.6 |
| | T5101024a | 0.9 | 9.8 | 4.3 | 11.5 | 2.0 | 74.8 |
| | Mean | 0.51 | 11.00 | 49.26 | 4.49 | 3.06 | 34.68 |
| | SD | 0.34 | 5.71 | 26.18 | 4.27 | 2.39 | 31.40 |

TABLE 19
Memory Phenotype of CD4⁺ and CD8⁺
TILs after REP with TransAct and Sequential
Use of G-Rex ® Device and Xuri ™ W25 Bioreactor

| | Sample ID | T_{N}/T_{SCM} | T_{CM} | T_{EM} | T_{EFF} |
|---|---|---|---|---|---|
| Proportion | T5101018 | 0.3 | 17.9 | 81.6 | 0.3 |
| of CD4⁺ | T5101015-1 | 5.5 | 28.4 | 62.3 | 3.8 |
| TILs (%) | T5101015-2 | 3.6 | 37.2 | 55.9 | 3.3 |
| | T5101015-3 | 4.0 | 49.5 | 44.6 | 1.9 |
| | T5101024a | 7.5 | 67.8 | 23.1 | 1.6 |
| | Mean | 4.16 | 40.16 | 53.50 | 2.19 |
| | SD | 2.67 | 19.32 | 21.66 | 1.41 |
| Proportion | T5101018 | 0.1 | 65.6 | 33.4 | 0.9 |
| of CD8⁺ | T5101015-1 | 2.6 | 16.9 | 74.0 | 6.5 |
| TILs (%) | T5101015-2 | 3.0 | 26.2 | 64.4 | 6.4 |
| | T5101015-3 | 4.8 | 29.7 | 57.1 | 8.3 |
| | T5101024a | 31.7 | 18.3 | 15.4 | 34.6 |
| | Mean | 8.45 | 31.34 | 48.86 | 11.32 |
| | SD | 13.11 | 19.88 | 23.98 | 13.31 |

Taken together, we have determined the culture parameters for optimal performance of Xuri™ REP. The TransAct REP process we developed with sequential use of a G-Rex® device and Xuri™ bioreactor can be applied for manufacturing of TIL cell therapy products that can easily meet the target cell dose while maintaining desired T cell phenotype.

Characterization of TILs from PD Runs by TCR Sequencing

We performed TCR beta chain sequencing of TILs from 2 donors to compare the repertoire diversity before (pre-REP) and after the manufacturing scale REP PD runs. The REP TILs displayed a large TCR overlap with their cognate pre-REP TILs (92% for T5101015-3 and 77% for T5101024a, respectively), implying that the TCR clones presented by pre-REP TILs were largely remained after the REP process. We also computed Morisita Index for each pre-REP and REP sample pair, which is a correlation analysis considering both the number and relative abundance of shared clones between the two samples. The value ranges from 0 to 1, and high values indicate high similarity between the overall repertoire of the two samples. The Morisita Indices were also relatively high (0.80 for T5101015 and 0.54 for T5101024, respectively), again suggesting that REP TILs from the PD runs may well preserve the repertoire diversity of pre-REP TILs. This is further supported by additional analyses of Simpson clonality and preservation of top 100 pre-REP clones after REP.

Functional Characterization of TILs from PD Runs

To provide evidence for the functionality of the REP TILs from TIL PD runs, we examined the cell activation and cytokine secretion of TILs from 3 donors after Xuri™ REP, in response to αCD3 stimulation. All the 3 PD products were polyfunctional, producing large amounts of IFN-γ, Granzyme B, and TNF-α upon αCD3 stimulation alone, although the absolute levels varied among the donors. The cytokine production could potentially be enhanced for some donors by the addition of IL-2. Consistently, surface expressions of 4-1BB and OX40 were upregulated by αCD3 stimulation alone, with considerably enhanced expression of the latter by the addition of IL-2, in both CD4⁺ and CD8⁺ populations. These data demonstrated that the REP TILs of C-TIL051 PD runs retain the functionality in response to T cell activation signal.

Characterization of TILs from Mock Runs

Based on the PD work described above, we established standard operation procedures. T5101034 and T5101035 fill scale mock runs were performed at a GMP facility. The REP products were tested. Characterization of the TIL final product is presented below.

TIL Yield, Viability, and T Cell Composition from Mock Runs

At the time of harvest, the REP yield from both mock runs reached our target dose of 1×10⁹ to 100×10⁹ cells (1.72×10⁹ and 5.95×10⁹ for T5101034 and T5101035, respectively; Table 20) with high viability (96.5% and 96.8% for T5101034 and T5101035, respectively; Table 20). The CD3⁺ percentage of the REP TILs were 86.98% and 97.42% for T5101034 and T5101035, respectively, among which 30.96% and 9.81% are CD8⁺ T cells, respectively (Table 20).

TABLE 20
TIL Yield, Viability, and T Cell Composition from Mock Runs

| Sample ID | Viability (%) | CD3⁺ (%) | CD3⁺ CD8⁺ (%) | Cell number |
|---|---|---|---|---|
| T5101034 | 96.5 | 86.98 | 30.96 | 1.72 × 10⁹ |
| T5101035 | 96.8 | 97.42 | 9.81 | 5.95 × 10⁹ |

T Cell Phenotype of C-TIL051 from Mock Runs

We examined the T cell activation and exhaustion markers, as well as memory phenotypes of the REP TIL products from mock runs. In CD4⁺ subset, surface expression of 4-1BB was minimal for both samples (0.20% and 0.87% for T5101034 and T5101035, respectively), whereas that of PD-1 was repeatedly detected (14.56% and 26.90% for T5101034 and T5101035, respectively; Table 21). In CD8⁺ subset, surface expression of 4-1BB was slightly higher (2.36% and 6.13% for T5101034 and T5101035, respectively), while that of PD-1 was lower for both samples (˜5%), as compared to CD4⁺ subset (Table 21). In addition, phenotype analysis showed that both TIL products were mainly composed of memory cells, with 19.93% and 22.35% at naïve or stem cell memory (T_{N}/T_{SCM}) stage, 29.15% and 19.72% at central memory (T_{CM}) stage, and 37.56% and 34.37% at effector memory (T_{EM}) stage, for T5101034 and T5101035, respectively (Table 22). The proportion of effector cells (T_{EFF}) was 13.36% and 23.56% for the two products, respectively (Table 22).

TABLE 21
Surface Expression of 4-1BB and PD-1 on
CD4⁺ and CD8⁺ TILs from Mock Runs

| | Sample ID | 4-1BB | PD-1 |
|---|---|---|---|
| Expression on | T5101034 | 0.20 | 14.56 |
| CD4⁺ TILs (%) | T5101035 | 0.87 | 26.90 |
| Expression on | T5101034 | 2.36 | 5.29 |
| CD8⁺ TILs (%) | T5101035 | 6.13 | 5.25 |

TABLE 22
Memory Phenotype of TILs from Mock Runs

Proportion of CD3⁺ TILs (%)

| Sample ID | T_{N}/T_{SCM} | T_{CM} | T_{EM} | T_{EFF} |
|---|---|---|---|---|
| T5101034 | 19.93 | 29.15 | 37.56 | 13.36 |
| T5101035 | 22.35 | 19.72 | 34.37 | 23.56 |

Functional Characterization of TILs from Mock Runs

We first performed IFN-γ release assay for functional characterization of the REP TIL product. After overnight stimulation with the anti-CD3/CD28-coupled Dynabeads, both products released large amount of IFN-γ (10.46 fg/cell and 16.15 fg/cell for T5101034 and T5101035, respectively; FIG. 17).

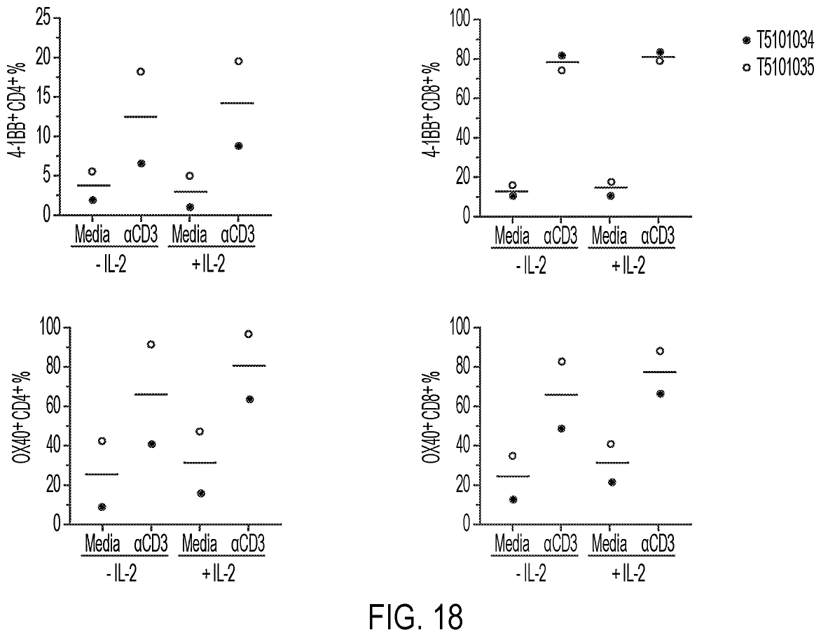

We also examined the surface expression of T cell activation markers by the REP TIL product after overnight stimulation with αCD3 antibody. The activation signal strongly induced the surface expression of both 4-1BB and OX40 in either CD4⁺ or CD8⁺ subsets of the two products (FIG. 18).

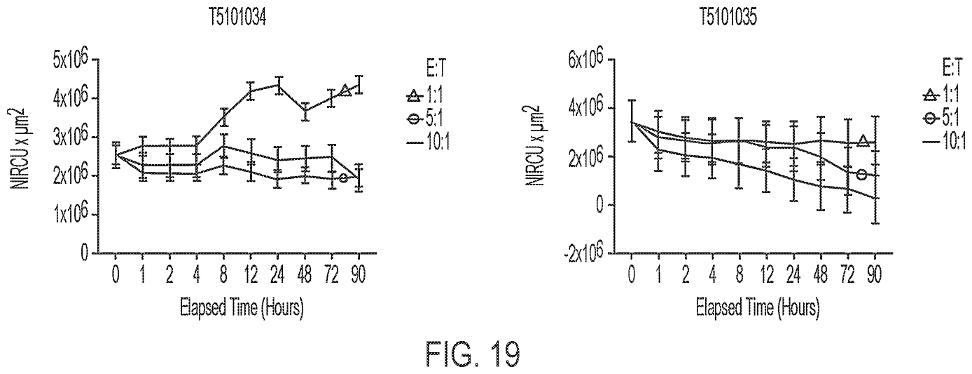

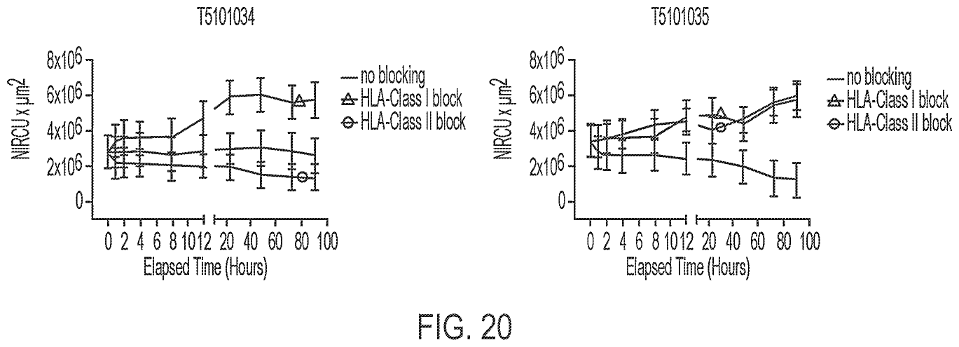

Furthermore, we evaluated the in vitro tumor-killing effect of the REP TILs from the two full scale mock runs on MOS generated from cognate tumor specimens. Characterization by morphology, immunohistochemistry staining of EpCAM, and immunofluorescent staining of EpCAM and pan-cytokeratin confirmed that both T5101034 and T5101035 MOS were of tumor origin. In subsequent potency assay, both products were able to eradicate MOS when co-cultured at higher effector to target ratios (5:1 and 10:1, FIG. 19), which was quantified by measuring the fluorescence intensity from remaining NIR680 dye-positive live tumor cells. In both cases, the effect could be detected as early as 8 hours of co-culture and became increasingly pronounced as time passed (FIG. 19). Importantly, addition of HLA class I blocking antibody in the co-culture completely abrogated the killing effect of T5101034 TIL product, while HLA Class II blocking showed no effect (FIG. 20, left), implying that the killing function was primarily mediated by CD8⁺ T cells. In the case of T5101035, addition of either HLA class I or class II blocking antibody could abrogate the killing effect (FIG. 20, right), implying the killing function required both CD4⁺ and CD8⁺ T cells. Despite the donor difference, the dependence on HLA recognition in both cases demonstrated that the tumor-killing functions of T5101034 TIL and T5101035 TILs are specific to the tumor cells.

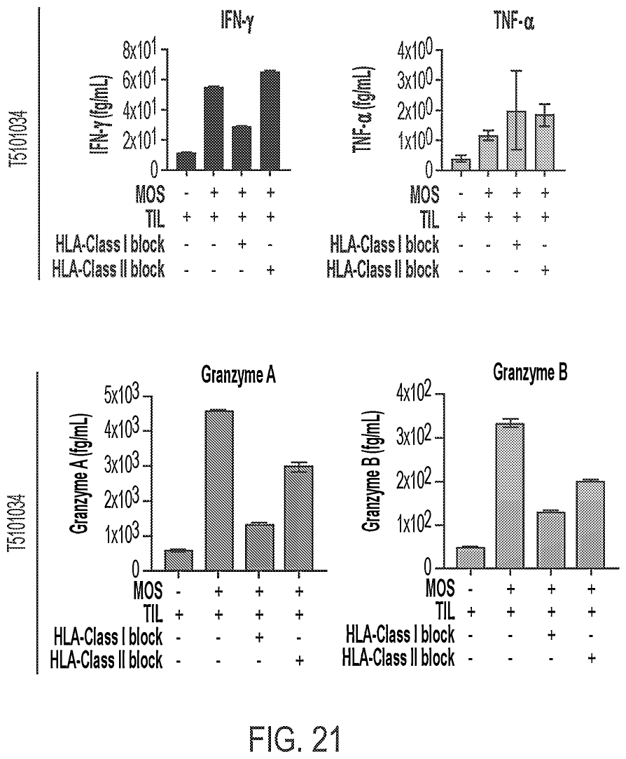

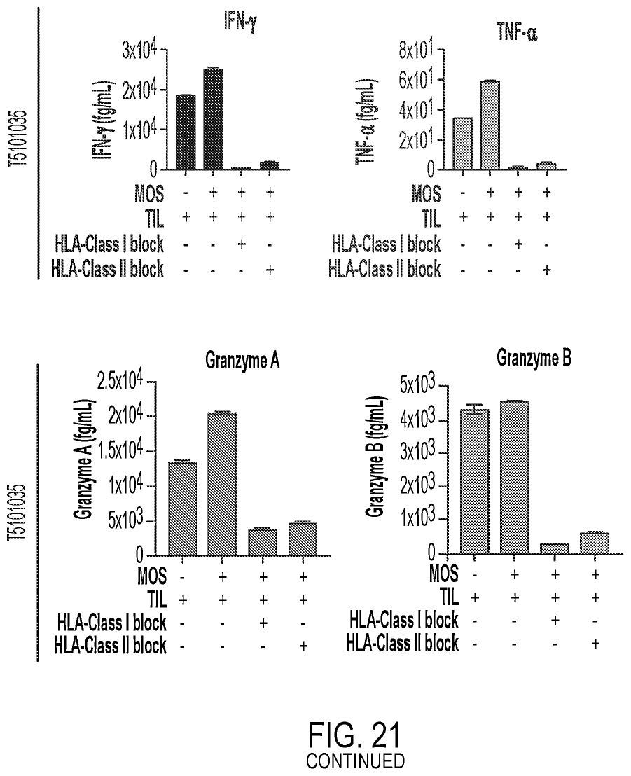

In addition, we collected the culture supernatants of the TILs and MOS co-cultures (E:T=5:1) at day 3 were collected for cytokine assay. Compared with TIL only control, the TILs and MOS co-culture groups had significantly increased levels of IFN-γ and Granzyme A in culture supernatants (FIG. 21). The level of Granzyme B in T5101034 TILs and MOS co-culture supernatant was also increased, so did that of TNF-α in T5101035 TILs and MOS co-culture supernatant (FIG. 21). The results agreed with the TIL tumor killing potency results described earlier. Moreover, adding HLA-Class I blocking antibody drastically decreased the cytokine levels in co-culture supernatants of TIL and MOS from both donors, while addition of HLA-Class II blocking antibody decreased the cytokine levels primarily in co-culture supernatant of T5101035 TILs and MOS (FIG. 21). Despite the donor difference, the dependence on HLA recognition in both cases demonstrated that the tumor-killing effects of TILs were through the TCR. Taken together, these data demonstrated that the TILs from the mock runs are highly potent in killing autologous tumor cells.

### CONCLUSIONS

We successfully developed a feeder-free TIL REP process with the αCD3/αCD28 nanoparticles. The TIL REP process can be started immediately after thawing of pre-REP cells. Depending on the live cell count of pre-REP TILs, different cell expansion device or system (i.e., G-Rex®10M, G-Rex®100M-CS, and Xuri™ W25 bioreactor) can be used for the REP process and the targeted therapeutic dose can be achieved. The T cell repertoire diversity is preserved in the TILs. The TILs also display desired T cell phenotypes, the ability to upregulate T-cell activation markers and to produce various cytokines in response to T cell activation signals. Furthermore, functional characterization of the TILs from mock runs demonstrated that the products are potent in killing autologous tumor cells.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

1. A method of expanding tumor-infiltrating lymphocytes (TILs), the method comprising:
(a) culturing a first population of cells in a first cell culture medium to generate a second population of cells, wherein the first population of cells is obtained from a tumor sample from a patient; and
(b) contacting the second population of cells with a polymeric matrix comprising anti-CD3 and anti-CD28 antibodies or fragments thereof in a second cell culture medium, to generate a third population of cells, wherein the second cell culture medium comprises about 100 IU/mL to about 8,000 IU/mL interleukin-2 (IL-2).

2. The method of claim 1, wherein the first cell culture medium comprises about 2,000 IU/mL to about 8,000 IU/mL IL-2.

3. The method of claim 1, wherein in step (a) the first population of cells is cultured for about 10 days to about 40 days.

4. The method of claim 3, wherein in step (a) the first population of cells is cultured for about 10 days to about 14 days.

5. The method of claim 1, wherein the second cell culture medium comprises about 500 IU/mL to about 4,000 IU/mL IL-2.

6-7. (canceled)

8. The method of claim 1, wherein in step (b) the contacting is for about 3 days to about 17 days.

9. The method of claim 1, wherein the third population of cells is at least 100-fold greater in number than the second population of cells.

10. (canceled)

11. The method of claim 1, wherein the tumor sample is from a solid tumor.

12. The method of claim 11, wherein the solid tumor comprises a sarcoma, hepatocellular carcinoma, glioma, head-neck cancer, bone cancer, brain cancer, breast cancer, cancer of the anus, cancer of the anal canal, cancer of the anorectum, cancer of the eye, cancer of the intrahepatic bile duct, cancer of the joints, cancer of the neck, cancer of the gallbladder, cancer of the pleura, cancer of the nose, cancer of the nasal cavity, cancer of the middle ear, cancer of the oral cavity, cancer of the vulva, colon cancer, esophageal cancer, cervical cancer, gastrointestinal cancer, hypopharynx cancer, larynx cancer, liver cancer, lung cancer, malignant mesothelioma, melanoma, nasopharynx cancer, ovarian cancer, pancreatic cancer, peritoneum cancer, omentum cancer, mesentery cancer, pharynx cancer, prostate cancer, rectal cancer, renal cancer, small intestine cancer, soft tissue cancer, stomach cancer, testicular cancer, thyroid cancer, ureter cancer, urinary bladder cancer, or combinations thereof.

13. (canceled)

14. A cell population enriched for, or expanded from, tumor-infiltrating lymphocytes, comprising one or more of the following:
(i) CD3⁺ CD8⁺ T cells at a percentage ranging from about 3% to about 88% of CD3⁺ cells,
(ii) CD3⁺ CD4⁺ T cells at a percentage ranging from about 10% to about 96% of CD3⁺ cells,
(iii) CD4⁺ T_{CM} T cells at a percentage ranging from about 50% to about 88% of CD4⁺ cells,
(iv) CD8⁺ T_{CM} T cells at a percentage ranging from about 28% to about 82% of CD8⁺ cells,
(v) CD4⁺ T_{EM} T cells at a percentage ranging from about 11% to about 49% of CD4⁺ cells, and
(vi) CD8⁺ T_{EM} T cells at a percentage ranging from about 11% to about 61% of CD8⁺ cells, wherein the cell population comprises no less than 70% of live cells, and wherein the cell population is generated from a tumor sample from a patient.

15. The cell population of claim 14, comprising no less than 80% CD3⁺ T cells in live cells.

16. The cell population of claim 14, comprising CD4⁺CD27⁺ T cells at a percentage ranging from about 10% to about 51% of CD4⁺ cells.

17. The cell population of claim 14, comprising CD8⁺CD27⁺ T cells at a percentage ranging from about 12% to about 72% of CD8⁺ cells.

18. The cell population of claim 14, comprising CD8⁺CD28⁺ T cells at a percentage ranging from about 34% to about 95% of CD8+ cells.

19. The cell population of claim 14, comprising CD4⁺CD28⁺ T cells at a percentage ranging from about 82% to about 100% of CD4⁺ cells.

20. The cell population of claim 14, comprising CD4⁺ 4-1BB⁺ T cells at a percentage ranging from about 0.2% to about 5.8% of CD4⁺ cells.

21. The cell population of claim 14, comprising CD8⁺ 4-1BB⁺ T cells at a percentage ranging from about 0.2% to about 11.6% of CD8⁺ cells.

22. The cell population of claim 14, comprising CD4⁺ LAG3⁺ T cells at a percentage ranging from about 0.2% to about 19.5% of CD4⁺ cells.

23. The cell population of claim 14, comprising CD8⁺ LAG3⁺ T cells at a percentage ranging from about 6% to about 51.2% of CD8⁺ cells.

24. The cell population of claim 14, comprising CD4⁺ PD1⁺ T cells at a percentage ranging from about 0.9% to about 31% of CD4⁺ cells.

25. The cell population of claim 14, comprising CD8⁺ PD1⁺ T cells at a percentage ranging from about 1% to about 18% of CD8⁺ cells.

26. The cell population of claim 14, comprising no greater than 10% CD56⁺ NK cells.

27. (canceled)

28. A method of expanding a cell population enriched for tumor-infiltrating lymphocytes, the method comprising:
(a) culturing cells obtained from a tumor sample from a patient;
(b) treating the cultured cells to generate a cell population enriched for tumor-infiltrating lymphocytes, the cell population comprising one or more of the following:
(i) CD3⁺ CD8⁺ T cells at a percentage ranging from about 3% to about 88% of CD3⁺ cells,
(ii) CD3⁺ CD4⁺ T cells at a percentage ranging from about 10% to about 96% of CD3⁺ cells,
(iii) CD4⁺ CD45RA⁻CD62L⁺ central memory T cells at a percentage ranging from about 50% to about 88% of CD4⁺ cells,
(iv) CD8⁺CD45RA⁻CD62L⁺ central memory T cells at a percentage ranging from about 28% to about 82% of CD8⁺ cells,
(v) CD4⁺CD45RA⁻CD62L⁻ effector memory T cells at a percentage ranging from about 11% to about 49% of CD4⁺ cells, and
(vi) CD8⁺ T CD45RA⁻CD62L⁻ effector memory T cells at a percentage ranging from about 11% to about 61% of CD8⁺ cells.

29-33. (canceled)

34. A method of treating a patient with cancer, the method comprising administering to the patient the cell population of claim 14.

35. (canceled)

36. The method of claim 34, wherein the cancer is melanoma, cervical cancer, lung cancer, colorectal cancer, breast cancer, or head and neck cancer.

37-38. (canceled)
